(19) United States
(12) Patent Application Publication
(10) Pub. No.: US 2026/0116983 A1
(43) Pub. Date: Apr. 30, 2026

(54) Anti-Urokinase-Type Plasminogen Activator Receptor Antibodies and Methods of Use

(71) Applicants: The Regents of the University of California, Oakland, CA (US); ShangPharma Innovation, Inc., South San Francisco, CA (US)

(72) Inventors: André Luiz Pinto Guedes Lourenço, San Francisco, CA (US); Charles S. Craik, San Francisco, CA (US); Markus Bohn, San Francisco, CA (US); Shireen Khan, South San Francisco, CA (US); Nitin Patel, South San Francisco, CA (US); Neha Yevalekar, South San Francisco, CA (US); Shih-Wei Chuo, San Francisco, CA (US); Michael Evans, San Francisco, CA (US)

(21) Appl. No.: 19/115,685

(22) PCT Filed: Oct. 5, 2023
(86) PCT No.: PCT/US2023/076131

§ 371 Date: Mar. 26, 2025

Related U.S. Application Data
(60) Provisional application No. 63/413,530, filed on Oct. 5, 2022.

(51) Int. Cl. C07K 16/28; A61K 39/00; A61K 47/68; A61P 35/00

(52) U.S. Cl. CPC C07K 16/2851; A61K 47/68031; A61K 47/6849; A61P 35/00; A61K 2039/505; C07K 2317/24; C07K 2317/732; C07K 2317/92

(57) ABSTRACT

Provided are antibodies that specifically bind to human urokinase-type plasminogen activator receptor (uPAR). In some instances, the antibodies are cross-reactive one or more non-human animal uPAR polypeptides, such as a non-human primate uPAR, e.g., a cynomolgus uPAR. Fusion proteins and conjugates comprising the antibodies of the present disclosure are also provided. Methods of using the antibodies, fusion proteins and conjugates of the present disclosure to treat a condition associated with uPAR expression and/or activity are also provided. In some embodiments, the condition associated with uPAR expression and/or activity is cancer. Non-limiting examples of such cancers include those characterized by cancer cells that express uPAR on the surface thereof, cancers characterized by stromal cells in the tumor microenvironment that express uPAR on the surface thereof, and/or the like.

### CROSS-REFERENCE TO RELATED APPLICATIONS

This application claims the benefit of U.S. Provisional Patent Application No. 63/413,530, filed Oct. 5, 2022, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, UCSF-667WO_SEQ_LIST, created on Oct. 4, 2023 and having a size of 53,857 bytes. The contents of the Sequence Listing XML are incorporated herein by reference in their entirety.

### INTRODUCTION

A key feature of tumor cells is their enhanced ability to degrade extracellular matrix (ECM), allowing tumor cell motility, invasion, and metastasis. The urokinase-type plasminogen activator receptor (uPAR) is an integral membrane protein tethered to the plasma membrane via a glycosylphosphatidylinositol (GPI) anchor. This well-studied receptor is involved in the binding of various partners, such as urokinase-type plasminogen activator (uPA), vitronectin (VN), and transmembrane receptors, to regulate a wide variety of cellular processes including extracellular proteolysis, angiogenesis, cell adhesion, migration, and downstream signaling events (1). Many studies have demonstrated that the overexpression of uPAR is tumor-specific (2,3), making it a prominent biomarker for identifying tumor aggressiveness (4-6) and an attractive target for cancer treatment (7), particularly breast cancer (8-11).

Growing evidence suggests that uPAR and HER2 are co-amplified in both in situ and metastatic breast cancer, and they work cooperatively for tumor progression towards the onset of a metastatic phenotype (12,13). Moreover, downregulation of uPAR using RNAi with an anti-HER2 antibody induces synergistic effects in inhibiting breast cancer cell growth, highlighting the potential of a combined therapy as an effective treatment for breast cancer (14). Although the clinical outcomes have shown that FDA-approved anti-HER2 antibodies are effective in metastatic HER2-positive breast cancer, several mechanisms of resistance to anti-HER2 therapy have been identified (15,16). In addition, HER2 is not an effective target for triple-negative breast cancer (TNBC) patients because of the absence of HER2 expression (9), so there is a need for the development of novel treatment strategies. Various groups have developed a series of antagonists, such as recombinant antibodies (rAbs), small molecules, and peptides, to block the interaction of uPAR with its partners (17-22). Some of these uPAR-targeted agents have also been designed as novel preclinical immunotherapeutics (17,23,24), diagnostic imaging tools (17,25,26), and drug delivery vehicles (24), validating uPAR as a potential therapeutic target.

### SUMMARY

Provided are antibodies that specifically bind to human urokinase-type plasminogen activator receptor (uPAR). In some instances, the antibodies are cross-reactive one or more non-human animal uPAR polypeptides, such as a non-human primate uPAR, e.g., a cynomolgus uPAR. Fusion proteins and conjugates comprising the antibodies of the present disclosure are also provided. Methods of using the antibodies, fusion proteins and conjugates of the present disclosure to treat a condition associated with uPAR expression and/or activity are also provided. In some embodiments, the condition associated with uPAR expression and/or activity is cancer. Non-limiting examples of such cancers include those characterized by cancer cells that express uPAR on the surface thereof, cancers characterized by stromal cells in the tumor microenvironment that express uPAR on the surface thereof, and/or the like.

### BRIEF DESCRIPTION OF THE FIGURES

FIG. 1A-1D: A) Mouse Immunization campaign pipeline. B) A workflow for accelerated discovery of cross-reactive anti-uPAR antibodies using the Beacon™ platform. C) Each ASC was cultured in individual nanopens to allow secreted antibodies to be accumulated. After culturing cells for 1-2 hours, anti-mouse IgG (H+L)-coated beads are imported into the channel along with fluorescently labeled uPAR (AF488-human suPAR, green; and AF647-cyno uPAR, red). As the secreted antibodies are immobilized on the beads, antigen recognition allows for a time-dependent accumulation of fluorescent signals immediately above each nanopen. D) The increased signal in AF488 and AF647 channels was observed between TO and T11, suggesting the antibodies were able to recognize human/cyno uPAR.

FIG. 2: Binding curves for initial antibodies to MDA-MB-231 cells expressing human uPAR on the cell surface. MFI indicates median fluorescence intensity.

FIG. 3A-3B: (A) Cross-reactivity profile of 12 antibody candidates evaluated by ELISA, showing they are cross-reactive to human and cyno uPAR, and 2G10 and 3C6 are specific binders for human uPAR. (B) Antibody candidates can prime NK-92® MI CD16a effector cells to enact ADCC against MDA-MB-231 cells in a dose-dependent manner. Two-way ANOVA followed by posthoc Tukey test reveals significant activity over relative human IgG1 (huIgG1). *=p≤0.001.

FIG. 4: Eight selected antibody candidates were able to induce dose-dependent cell death in MBA-MB-231 in the presence of human PBMCs from three healthy donors. Two-way ANOVA followed by posthoc Tukey test reveals significant activity over relative human IgG1 (huIgG1). *=p≤0.05, **=p≤0.001.

FIG. 5A-5C: A) Dose-dependent cytotoxicity was observed for candidates 3159, 8163, 11857, and 3595 in the presence of an anti-human Fc Fab conjugated to cytotoxic MMAE through a cathepsin-cleavable linker. B) Therapeutic efficacy of novel antibody candidates was determined in an orthotopic mice model of human breast cancer using MDA-MB-231 cells. Animals showed reduced tumor size in comparison to untreated controls, and significant tumor growth suppression was observed in animals treated with 11857 after 21 days of treatment. C) Significant impairment of tumor growth rates was observed for antibody-treated animals throughout the 30-day treatment, whereas 11857 was the most active agent. Data are shown as mean±standard deviation. Statistical analysis as Two-way ANOVA, with a posthoc multiple comparisons using Dunnett's test. *p<0.05; **p<0.01; ***p<0.001.

FIG. 6A-6C: A) Molecular surface representation of human uPAR-ATF-SMB complex. The uPA N-terminal fragment (ATF) is shown as a ribbon diagram in gray, and the vitronectin (VN) SMB domain is shown as a ribbon diagram in blue. (PDB ID: 3BT1). Mutation variants between human and cyno uPAR are highlighted in yellow. B) BLI traces identify non-overlapping epitopes between each lead antibody and 2G10, which binds to the uPA recognition site. The further association step demonstrates the competitive blocking of VN binding by each antibody candidate. C) BLI competition assay reveals candidates 8163 and 3159 have distinct binding sites, and candidate 11857 has a partially overlapping epitope with candidates 8163 and 3159.

FIG. 7: Proposed binding model for novel antibody candidates to uPAR, highlighting their inhibitory effects for vitronectin binding and distinct binding epitopes compared to 2G10. Antibodies 3159 and 8163 recognize distinct epitopes on uPAR, and 11857 has a partial overlapping epitope with antibodies 3159 and 8163.

FIG. 8A-8B: A) Recombinant human suPAR expressed and characterized by SDS-PAGE and immunoblot. B) Further characterization by LC-MS/MS showed 59.7% coverage of the complete protein sequence (SEQ ID NO:55).

FIG. 9: Antibody titers from each animal monitored throughout a 60-day immunization campaign using recombinant human uPAR as an immunogen, showing the production of anti-uPAR antibodies.

FIG. 10: Eight selected lead antibody candidates were able to block the adhesion of MDA-MB-231 cells to vitronectin in a dose-response manner.

FIG. 11: BLI competition assay between candidate 3159 and vitronectin in opposing order shows the ability of 3159 for blocking vitronectin binding to uPAR.

FIG. 12: BLI competition assay for lead antibodies and 3C6, which was discovered by phage-display, shows they have distinct binding epitopes.

FIG. 13: PET/CT slices taken at different time points from mice dosed with 89Zr-DFO-3159 antibodies.

FIG. 14: Maximum intensity projections taken at different time points from mice dosed with 89Zr-DFO-3159 antibodies.

FIG. 15: PET/CT slices taken at different time points from mice dosed with 89Zr-DFO-11857 antibodies.

FIG. 16: Maximum intensity projections taken at different time points from mice dosed with 89Zr-DFO-11857 antibodies.

FIG. 17: Tumor time activity curves including SUVmean data acquired from 4 tumors in the 3159 cohort and 3 tumors in the 11857 cohort.

FIG. 18: SUVmean data acquired by region of interest analysis on the tumor and various normal tissues from the mice in the 3159 cohort.

FIG. 19: SUVmean data acquired by region of interest analysis on the tumor and various normal tissues from the mice in the 11857 cohort.

FIG. 20: Antitumor assessment depicting a fold change in volume for UMUC3 tumors.

FIG. 21. Antitumor assessment depicting a volume change for UMUC3 tumors.

### DETAILED DESCRIPTION

Before the antibodies and methods of the present disclosure are described in greater detail, it is to be understood that the antibodies and methods are not limited to particular embodiments described, as such may, of course, vary. It is also to be understood that the terminology used herein is for the purpose of describing particular embodiments only, and is not intended to be limiting, since the scope of the antibodies and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the antibodies and methods. The upper and lower limits of these smaller ranges may independently be included in the smaller ranges and are also encompassed within the antibodies and methods, subject to any specifically excluded limit in the stated range. Where the stated range includes one or both of the limits, ranges excluding either or both of those included limits are also included in the antibodies and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the antibodies and methods belong. Although any antibodies and methods similar or equivalent to those described herein can also be used in the practice or testing of the antibodies and methods, representative illustrative antibodies and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present antibodies and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the antibodies and methods, which are, for clarity, described in the context of separate embodiments, may also be provided in combination in a single embodiment. Conversely, various features of the antibodies and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present antibodies and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Anti-uPAR Antibodies

The present disclosure provides anti-urokinase-type plasminogen activator receptor (uPAR) antibodies. uPAR (UniProt Q03405-human), also known as CD87, is encoded by the PLAUR gene and belongs to the lymphatic antigen-6 superfamily. uPAR was first identified as the cell surface receptor for urokinase plasminogen activator (uPA). The mature uPAR molecule is a single-chain membrane glycoprotein receptor composed of 313 amino acid residues and is anchored to the cell membrane by a glycosylphosphatidylinositol (GPI) linkage; it contains 3 homologous domains, D1, D2 and D3, with a total molecular weight of 55-60 kDa. uPAR mediates a variety of biological processes, such as plasminogen activation, proteolysis, cellular signal transduction and adhesion. Under normal physiological conditions, uPAR is usually expressed at a low level. In the processes of tissue remodeling, wound healing, inflammation and embryogenesis, uPAR is transiently expressed at high levels and participates in the processes of extracellular matrix (ECM) degradation, thrombolysis, cell invasion and migration.

uPAR has multiple functional roles associated with tumor progression, including tumor proliferation and apoptosis, metastasis, angiogenesis, multi-drug resistance (MDR) and prognosis. An analysis of tumor samples has shown high uPAR expression in most solid tumor tissues, including but not limited to, breast, lung, bladder, ovarian, prostate, liver, colon, pancreatic and gastric cancer, as well as gliomas and several hematologic malignancies. Moreover, uPAR is expressed at high levels on stromal cells in the tumor microenvironment, such as vascular endothelial cells, tumor-related fibroblasts and tumor-related macrophages, and its expression level is closely related to tumor aggressiveness and the survival of patients with tumors.

In certain embodiments, an antibody of the present disclosure specifically binds to human urokinase-type plasminogen activator receptor (uPAR) and competes for binding to human uPAR with an antibody having one, two, three, four, five, or all six complementarity determining regions (CDRs) of one or more of the anti-uPAR antibodies designated herein as antibody 3159, 8163, 11857, or 3595. In some embodiments, such an antibody comprises one, two, three, four, five, or all six CDRs of an antibody designated herein as antibody 3159, 8163, 11857, or 3595. In some embodiments, such antibodies comprise a variable heavy chain (V_{H}) polypeptide and/or a variable light chain (V_{L}) polypeptide having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the Vu and/or the V_{L} of an antibody designated herein as antibody 3159, 8163, 11857, or 3595.

Antibodies 3159, 8163, 11857, and 3595 were selected among a large number of identified anti-human uPAR antibodies, based at least in part on their ability to cross-react with cynomolgus uPAR. The cynomolgus monkeys (cyno) are genetically similar to human compared to other species and are the most relevant non-human primate model for conducting pre-clinical studies in the development of antibody drugs. Moreover, as demonstrated in the Experimental section below, these unique cross-reactive antibodies exhibit antibody-dependent cellular cytotoxicity (ADCC), ADC cytotoxicity, and inhibitory effects on cell adhesion against human cancer cells. Also as demonstrated herein, these antibodies exhibit therapeutic efficacy in reducing tumor growth in an orthotopic animal model of human cancer, and a binding model of these antibodies is provided showing their binding epitopes that lead to unique activities against uPAR.

The amino acid sequences of the V_{H} polypeptides and V_{L} polypeptides of the 3159, 8163, 11857, and 3595 antibodies are provided in Table 1 below. CDR sequences defined according to Kabat are underlined.

TABLE 1
Amino Acid and Nucleotide Sequences

| | | SEQ ID |
|---|---|---|
| Antibody 3159 V_{H} | QVTLKESGPGILQSSLTLSLTCSLSGFSLSTSGMGVSWIRQPSGKGLE | 1 |
| Amino Acid | WLAHIYWDDDKRYNPSLKTRLTISKDASRDQVFLKIISVDTADTGTYY | |
| | CGRRVRNYFSGTSYWYFDVWGTGTTVTVSS | |
| Antibody 3159 | TSGMGVS | 2 |
| V_{H} CDR1 | | |
| Antibody 3159 | HIYWDDDKRYNPSLKT | 3 |
| V_{H} CDR2 | | |
| Antibody 3159 | RVRNYFSGTSYWYFDV | 4 |
| V_{H} CDR3 | | |
| Antibody 3159 V_{L} | DVLMTQTPLSLSVSLGDQASISCRSSQNILHRTGNTYLEWYLQKPGQ | 5 |
| Amino Acid | SPKLLIYKVSNRFSGVPDRFSGSGSGTDFTLKISRLEAEDLGVYYCFQ | |
| | GSYVPFTFGSGTKLEMK | |
| Antibody 3159 | RSSQNILHRTGNTYLE | 6 |
| V_{L} CDR1 | | |
| Antibody 3159 | KVSNRFS | 7 |
| V_{L} CDR2 | | |
| Antibody 3159 | FQGSYVPFT | 8 |
| V_{L} CDR3 | | |
| Antibody 8163 V_{H} | EVQLVESGGGLVQPGESLTLSCDSNEFDFPSHDMSWVRKTPEKRLE | 9 |
| Amino Acid | LVAAIDSDGGLTYYSNSRERRFIISRDNTKNTLYLQMRSLRSEDTALY | |
| | YCARRRASYWYFDVWGTGTTVTVSS | |
| Antibody 8163 | SHDMS | 10 |
| V_{H} CDR1 | | |
| Antibody 8163 | AIDSDGGLTYYSNSRER | 11 |
| V_{H} CDR2 | | |
| Antibody 8163 | RRASYWYFDV | 12 |
| V_{H} CDR3 | | |
| Antibody 8163 V_{L} | DILLTQSPAILPVSPGERVSFSCRASQNIGTSIHWYQQRTNGSPRLLIK | 13 |
| Amino Acid | YASESISGIPSRFSGSGSGTDFTLSINGVESEDIGDYYCQQSNSWPTF | |
| | GGGTKLEIK | |
| Antibody 8163 | RASQNIGTSIH | 14 |
| V_{L} CDR1 | | |
| Antibody 8163 | YASESIS | 15 |
| V_{L} CDR2 | | |
| Antibody 8163 | QQSNSWPT | 16 |
| V_{L} CDR3 | | |
| Antibody 11857 | EVQLRQSGPELVKPGASVKISCKASGYTFTDYYMNWVKQSHEKSLE | 17 |
| V_{H} Amino Acid | WIGNINPNNGGTDYNQKFKGKATLTVDKSSSTAYMELRSLTSEDSAV | |
| | YYCARSYGSRFPYWGLGTLVTVSA | |
| Antibody 11857 | DYYMN | 18 |
| V_{H} CDR1 | | |
| Antibody 11857 | NINPNNGGTDYNQKFKG | 19 |
| V_{H} CDR2 | | |
| Antibody 11857 | SYGSRFPY | 20 |
| V_{H} CDR3 | | |
| Antibody 11857 V_{L} | DIQMTQTTSSLSASLGDRVTISCRASQDITNYLSWYQQKPDGTVKLLI | 21 |
| Amino Acid | FYTAVLQSGFPLRFSGSGSGADYSLTISNLEQEDFATYFCQQGHTLP | |
| | WTFGGGTKLEIK | |
| Antibody 11857 | RASQDITNYLS | 22 |
| V_{L} CDR1 | | |
| Antibody 11857 | YTAVLQS | 23 |
| V_{L} CDR2 | | |
| Antibody 11857 | QQGHTLPWT | 24 |
| V_{L} CDR3 | | |
| Antibody 3595 V_{H} | EVQLRQSGPELVKPGASVKISCKASGYTFTDYYMNWVKQSHEKSLE | 17 |
| Amino Acid | WIGNINPNNGGTDYNQKFKGKATLTVDKSSSTAYMELRSLTSEDSAV | |
| | YYCARSYGSRFPYWGLGTLVTVSA | |
| Antibody 3595 | DYYMN | 18 |
| V_{H} CDR1 | | |
| Antibody 3595 | NINPNNGGTDYNQKFKG | 19 |
| V_{H} CDR2 | | |
| Antibody 3595 | SYGSRFPY | 20 |
| V_{H} CDR3 | | |
| Antibody 3595 V_{L} | DIQMTQTTSSLSASLGDRVTISCRASQDITNYLSWYQQKPDGTVKLLI | 25 |
| Amino Acid | FYTSFLQSGVPLRFSGSGSGADYSLTISNLEQEDFATYFCQQGHTLP | |
| | WTFGGGTKLEIK | |
| Antibody 3595 | RASQDITNYLS | 22 |
| V_{L} CDR1 | | |
| Antibody 3595 | YTSFLQS | 26 |
| V_{L} CDR2 | | |
| Antibody 3595 | QQGHTLPWT | 24 |
| V_{L} CDR3 | | |
| Antibody 3159 V_{H} | CAGGTAACTCTGAAAGAGTCTGGCCCTGGGATATTGCAGTCCTCC | 27 |
| Nucleotide | CTGACCCTCAGTCTGACTTGTTCTTTATCTGGGTTTTCACTGAGCA | |
| | CTTCTGGCATGGGTGTGAGCTGGATTCGTCAGCCTTCAGGAAAGG | |
| | GTCTGGAGTGGCTGGCACACATTTACTGGGATGATGACAAACGCT | |
| | ATAATCCATCCCTGAAGACCCGGCTAACAATCTCCAAGGATGCCT | |
| | CCAGAGACCAGGTTTTCCTCAAGATCATCAGTGTGGACACTGCAG | |
| | ACACTGGCACATACTATTGTGGTCGAAGAGTTAGGAATTATTTCTC | |
| | CGGTACTAGTTACTGGTACTTCGATGTCTGGGGCACAGGGACCAC | |
| | GGTCACCGTCTCCTCA | |
| Antibody 3159 V_{L} | GATGTTTTGATGACCCAAACTCCACTCTCCCTGTCTGTCAGTCTTG | 28 |
| Nucleotide | GAGATCAAGCCTCCATCTCTTGCAGATCTAGTCAGAACATTCTACA | |
| | TCGTACTGGAAACACCTATTTAGAATGGTACCTGCAGAAACCAGG | |
| | CCAGTCTCCAAAGCTCCTGATCTACAAAGTTTCCAACCGATTTTCT | |
| | GGGGTCCCAGATAGGTTCAGTGGCAGTGGATCAGGGACAGATTT | |
| | CACACTCAAGATCAGTAGATTGGAGGCTGAGGATCTGGGAGTTTA | |
| | TTACTGCTTTCAAGGTTCATATGTTCCTTTCACGTTCGGCTCGGGG | |
| | ACAAAGTTGGAAATGAAA | |
| Antibody 8163 V_{H} | GAGGTACAACTGGTGGAGTCTGGGGGAGGCTTAGTGCAGCCTGG | 29 |
| Nucleotide | AGAGTCCCTGACACTCTCCTGTGATTCCAATGAATTCGATTTCCCT | |
| | TCCCATGACATGTCTTGGGTCCGCAAGACTCCGGAGAAGAGGCTG | |
| | GAGTTGGTCGCAGCCATTGATAGTGATGGTGGTCTCACCTACTAT | |
| | TCAAACAGTAGGGAGAGACGATTCATCATCTCCAGAGACAATACC | |
| | AAGAATACCCTGTACCTTCAAATGAGAAGTCTGAGGTCTGAGGAC | |
| | ACAGCCTTATATTACTGTGCAAGACGGAGGGCCTCCTACTGGTAC | |
| | TTCGATGTCTGGGGCACAGGGACCACTGTCACCGTCTCCTCAG | |
| Antibody 8163 V_{L} | GACATCTTGCTGACTCAGTCTCCAGCCATCCTGCCTGTGAGTCCA | 30 |
| Nucleotide | GGAGAAAGAGTCAGTTTCTCCTGCAGGGCCAGTCAAAACATTGGC | |
| | ACAAGTATACACTGGTATCAGCAAAGAACAAATGGTTCTCCAAGGC | |
| | TTCTCATAAAGTATGCCTCTGAGTCTATTTCTGGGATCCCTTCCAG | |
| | ATTTAGTGGCAGTGGATCAGGGACAGATTTTACTCTTAGCATCAAC | |
| | GGTGTGGAGTCTGAAGATATTGGAGATTATTACTGTCAACAAAGTA | |
| | ATAGCTGGCCAACGTTCGGAGGGGGGACCAAGCTGGAAATTAAA | |
| Antibody 11857 | GAGGTCCAGCTGCGACAATCTGGACCTGAGCTGGTGAAGCCTGG | 31 |
| V_{H} Nucleotide | GGCTTCAGTGAAGATATCCTGTAAGGCTTCTGGATACACGTTCACT | |
| | GACTATTACATGAACTGGGTGAAACAGAGCCATGAAAAGAGCCTT | |
| | GAGTGGATTGGAAATATTAATCCTAACAATGGTGGTACTGACTACA | |
| | ACCAGAAGTTCAAGGGCAAGGCCACATTGACTGTCGACAAGTCCT | |
| | CCAGTACAGCCTACATGGAGCTCCGCAGCCTGACATCTGAGGACT | |
| | CTGCAGTCTATTACTGTGCAAGATCTTACGGTAGTCGGTTTCCTTA | |
| | CTGGGGCCTAGGGACTCTGGTCACTGTGTCTGCA | |
| Antibody 11857 V_{L} | GATATCCAGATGACACAGACTACATCCTCCCTGTCTGCCTCTCTG | 32 |
| Nucleotide | GGAGACAGAGTAACCATCAGTTGCAGGGCAAGTCAGGACATTACC | |
| | AATTATTTAAGTTGGTATCAGCAGAAACCAGATGGAACTGTTAAAC | |
| | TCCTGATCTTTTATACAGCAGTTTTACAGTCAGGATTCCCATTAAG | |
| | GTTCAGTGGCAGTGGGTCTGGAGCAGATTATTCTCTCACCATTAG | |
| | CAACCTGGAGCAAGAAGATTTTGCCACTTACTTTTGCCAACAGGGT | |
| | CATACGCTTCCGTGGACGTTTGGTGGAGGCACCAAGCTGGAAATC | |
| | AAA | |
| Antibody 3595 V_{H} | GAGGTACAGCTGCGACAATCTGGACCTGAGCTGGTGAAGCCTGG | 33 |
| Nucleotide | GGCTTCAGTGAAGATATCCTGTAAGGCTTCTGGATACACGTTCACT | |
| | GACTATTACATGAACTGGGTGAAACAGAGCCATGAAAAGAGCCTT | |
| | GAGTGGATTGGAAATATTAATCCTAACAATGGTGGTACTGACTACA | |
| | ACCAGAAGTTCAAGGGCAAGGCCACATTGACTGTCGACAAGTCCT | |
| | CCAGTACAGCCTACATGGAGCTCCGCAGCCTGACATCTGAGGACT | |
| | CTGCAGTCTATTACTGTGCAAGATCTTACGGTAGTCGGTTTCCTTA | |
| | CTGGGGCCTAGGGACTCTGGTCACTGTGTCTGCA | |
| Antibody 3595 V_{L} | GATATCCAGATGACACAGACTACATCCTCCCTGTCTGCCTCTCTG | 34 |
| Nucleotide | GGAGACAGAGTCACCATCAGTTGCAGGGCAAGTCAGGACATTACC | |
| | AATTATTTAAGTTGGTATCAGCAGAAACCAGATGGAACTGTTAAAC | |
| | TCCTGATCTTTTATACATCATTTTTACAGTCAGGAGTCCCATTAAGG | |
| | TTCAGTGGCAGTGGGTCTGGAGCAGATTATTCTCTCACCATTAGC | |
| | AACCTGGAGCAAGAAGATTTTGCCACTTACTTTTGCCAACAGGGTC | |
| | ATACGCTTCCGTGGACGTTTGGTGGAGGCACCAAGCTGGAAATCA | |
| | AA | |
| Antibody 11857 | EVQLVQSGAEVKKPGATVKISCKVSGYTFTDYYMNWVQQAPGKSLE | 35 |
| HC1 (humanized) | WMGNINPNNGGTDYNQKFQGRVTITADKSTSTAYMELSSLRSEDTA | |
| Amino Acid | VYYCARSYGSRFPYWGLGTLVTVSS | |
| Antibody 11857 | DYYMN | 18 |
| HC1 (humanized) | | |
| CDR1 | | |
| Antibody 11857 | NINPNNGGTDYNQKFQG | 39 |
| HC1 (humanized) | | |
| CDR2 | | |
| Antibody 11857 | SYGSRFPY | 20 |
| HC1 (humanized) | | |
| CDR3 | | |
| Antibody 11857 | EVQLVQSGAEVKKPGASVKVSCKASGYTFTDYYMNWVRQAPGKSLE | 36 |
| HC2 (humanized) | WMGNINPNNGGTDYSQKFQGRVTITVDTSASTAYMELSSLRSEDTAV | |
| Amino Acid | YYCARSYGSRFPYWGLGTLVTVSS | |
| Antibody 11857 | DYYMN | 18 |
| HC2 (humanized) | | |
| CDR1 | | |
| Antibody 11857 | NINPNNGGTDYSQKFQG | 37 |
| HC2 (humanized) | | |
| CDR2 | | |
| Antibody 11857 | SYGSRFPY | 20 |
| HC2 (humanized) | | |
| CDR3 | | |
| Antibody 11857 | EVQLVQSGAEVKKPGASVKVSCKASGYTFTDYYMNWVRQAPGKSLE | 38 |
| HC3 (humanized) | WMGNINPNNGGTDYNQKFQGRVTLTVDTSISTAYMELSRLRSDDTV | |
| Amino Acid | VYYCARSYGSRFPYWGLGTLVTVSS | |
| Antibody 11857 | DYYMN | 18 |
| HC3 (humanized) | | |
| CDR1 | | |
| Antibody 11857 | NINPNNGGTDYNQKFQG | 39 |
| HC3 (humanized) | | |
| CDR2 | | |
| Antibody 11857 | SYGSRFPY | 20 |
| HC3 (humanized) | | |
| CDR3 | | |
| Antibody 11857 | DIQMTQSPSSLSASVGDRVTITCRASQDITNYLSWYQQKPGGTVKLLL | 40 |
| LC1 (humanized) | YYTAVLQSGFPSRFSGSGSGADYTLTISSLQPEDFATYYCQQGHTLP | |
| Amino Acid | WTFGGGTKVEIK | |
| Antibody 11857 | RASQDITNYLS | 22 |
| LC1 (humanized) | | |
| CDR1 | | |
| Antibody 11857 | YTAVLQS | 23 |
| LC1 (humanized) | | |
| CDR2 | | |
| Antibody 11857 | QQGHTLPWT | 24 |
| LC1 (humanized) | | |
| CDR3 | | |
| Antibody 11857 | DIQMTQSPSSLSASVGDRVTITCRASQDITNYLSWYQQKPGGTVKLLI | 41 |
| LC2 (humanized) | FYTAVLQSGFPSRFSGSGSGADFTLTISSLQPEDVATYYCQQGHTLP | |
| Amino Acid | WTFGGGTKVEIK | |
| Antibody 11857 | RASQDITNYLS | 22 |
| LC2 (humanized) | | |
| CDR1 | | |
| Antibody 11857 | YTAVLQS | 23 |
| LC2 (humanized) | | |
| CDR2 | | |
| Antibody 11857 | QQGHTLPWT | 24 |
| LC2 (humanized) | | |
| CDR3 | | |
| Antibody 11857 | DIQMTQSPSSLSASVGDRVTITCRASQDITNYLSWFQQKPGGTVKLLI | 42 |
| LC3 (humanized) | YYTAVLQSGFPSRFSGSGSGADFTLTISSLQPEDFATYYCQQGHTLP | |
| Amino Acid | WTFGGGTKVEIK | |
| Antibody 11857 | RASQDITNYLS | 22 |
| LC3 (humanized) | | |
| CDR1 | | |
| Antibody 11857 | YTAVLQS | 23 |
| LC3 (humanized) | | |
| CDR2 | | |
| Antibody 11857 | QQGHTLPWT | 24 |
| LC3 (humanized) | | |
| CDR3 | | |
| Antibody 3159 | QVTLRESGPALVKPTQTLTLTCTLSGFSLSTSGMGVSWIRQPPGKAL | 43 |
| HC1 (humanized) | EWLAHIYWDDDKRYSTSLKTRLTISKDASKDQVVLTMTNMDTVDTAT | |
| Amino Acid | YYCGRRVRNYFSGTSYWYFDVWGTGTLVTVSS | |
| Antibody 3159 | TSGMGVS | 2 |
| HC1 (humanized) | | |
| CDR1 | | |
| Antibody 3159 | HIYWDDDKRYSTSLKT | 44 |
| HC1 (humanized) | | |
| CDR2 | | |
| Antibody 3159 | RVRNYFSGTSYWYFDV | 4 |
| HC1 (humanized) | | |
| CDR3 | | |
| Antibody 3159 | QVTLKESGPVLVKPTETLTLTCTVSGFSLSTSGMGVSWIRQPPGKGL | 45 |
| HC2 (humanized) | EWLAHIYWDDDKRYSPSLKSRLTISKDASKDQVVLTMTNMDTVDTAT | |
| Amino Acid | YYCGRRVRNYFSGTSYWYFDVWGTGTLVTVSS | |
| Antibody 3159 | TSGMGVS | 2 |
| HC2 (humanized) | | |
| CDR1 | | |
| Antibody 3159 | HIYWDDDKRYSPSLKS | 46 |
| HC2 (humanized) | | |
| CDR2 | | |
| Antibody 3159 | RVRNYFSGTSYWYFDV | 4 |
| HC2 (humanized) | | |
| CDR3 | | |
| Antibody 3159 | QITLKESGPTLVKPTQTLTLTCTLSGFSLSTSGMGVSWIRQPPGKALE | 47 |
| HC3 (humanized) | WLAHIYWDDDKRYNPSLKSRLTITKDASKDQVVLTMTNMDTVDTATY | |
| Amino Acid | YCGRRVRNYFSGTSYWYFDVWGTGTLVTVSS | |
| Antibody 3159 | TSGMGVS | 2 |
| HC3 (humanized) | | |
| CDR1 | | |
| Antibody 3159 | HIYWDDDKRYNPSLKS | 48 |
| HC3 (humanized) | | |
| CDR2 | | |
| Antibody 3159 | RVRNYFSGTSYWYFDV | 4 |
| HC3 (humanized) | | |
| CDR3 | | |
| Antibody 3159 | DIVMTQTPLSLSVTPGQPASISCKSSQNILHRTGNTYLEWYLQKPGQP | 49 |
| LC1 (humanized) | PQLLIYKVSNRFSGVPDRFSGSGSGTDFTLKISRVEAEDVGVYYCFQ | |
| Amino Acid | GSYVPFTFGQGTKLEIK | |
| Antibody 3159 | KSSQNILHRTGNTYLE | 50 |
| LC1 (humanized) | | |
| CDR1 | | |
| Antibody 3159 | KVSNRFS | 7 |
| LC1 (humanized) | | |
| CDR2 | | |
| Antibody 3159 | FQGSYVPFT | 8 |
| LC1 (humanized) | | |
| CDR3 | | |
| Antibody 3159 | DVVMTQSPLSLPVTLGQPASISCRSSQNILHRTGNTYLEWFQQRPGQ | 51 |
| LC2 (humanized) | SPRLLIYKVSNRFSGVPDRFSGSGSGTDFTLKISRVEAEDVGVYYCFQ | |
| Amino Acid | GSYVPFTFGQGTKLEIK | |
| Antibody 3159 | RSSQNILHRTGNTYLE | 6 |
| LC2 (humanized) | | |
| CDR1 | | |
| Antibody 3159 | KVSNRFS | 7 |
| LC2 (humanized) | | |
| CDR2 | | |
| Antibody 3159 | FQGSYVPFT | 8 |
| LC2 (humanized) | | |
| CDR3 | | |
| Antibody 3159 | DIVMTQSPLSLPVTPGEPASISCRSSQNILHRTGNTYLDWYLQKPGQS | 52 |
| LC3 (humanized) | PQLLIYKVSNRFSGVPDRFSGSGSGTDFTLKISRVEAEDVGVYYCFQ | |
| Amino Acid | GSYVPFTFGQGTKLEIK | |
| Antibody 3159 | RSSQNILHRTGNTYLD | 53 |
| LC3 (humanized) | | |
| CDR1 | | |
| Antibody 3159 | KVSNRFS | 7 |
| LC3 (humanized) | | |
| CDR2 | | |
| Antibody 3159 | FQGSYVPFT | 8 |
| LC3 (humanized) | | |
| CDR3 | | |

According to some embodiments, an antibody of the present disclosure specifically binds human uPAR and comprises—or competes for binding to human uPAR with an antibody comprising—one, two, three, four, five, or all six CDRs of the antibody designated herein as antibody 3159. CDR sequences may be defined according to Kabat. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 3159; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 3159; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 3159.

In certain embodiments, an antibody of the present disclosure specifically binds human uPAR and comprises—or competes for binding to human uPAR with an antibody comprising-one, two, three, four, five, or all six CDRs of the antibody designated herein as antibody 8163. CDR sequences may be defined according to Kabat. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 8163; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 8163; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 8163.

According to some embodiments, an antibody of the present disclosure specifically binds human uPAR and comprises—or competes for binding to human uPAR with an antibody comprising—one, two, three, four, five, or all six CDRs of the antibody designated herein as antibody 11857. CDR sequences may be defined according to Kabat. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 11857; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 11857; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 11857.

According to some embodiments, an antibody of the present disclosure specifically binds human uPAR and comprises—or competes for binding to human uPAR with an antibody comprising—one, two, three, four, five, or all six CDRs of the antibody designated herein as antibody 3595. CDR sequences may be defined according to Kabat. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 3595; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 3595; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 3595.

According to some embodiments, the CDRs are defined according to the Kabat numbering system. In certain embodiments, the CDRs may be defined according to the IMGT numbering system.

In certain embodiments, antibody variants having one or more amino acid substitutions relative to a V_{H} and/or V_{L} amino acid sequence set forth in Table 1 are provided. Sites of interest for substitutional mutagenesis include one or more CDRs and/or one or more framework regions (FRs). Conservative substitutions are shown in the following table under the heading of “preferred substitutions.” More substantial changes are provided in the following table under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, improved developability, improved manufacturability, and/or the like.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Methods are available for measuring the affinity of an anti-human uPAR antibody for human uPAR using direct binding or competition binding assays. In a direct binding assay, the equilibrium binding constant (K_{D}) may be measured using a candidate anti-human uPAR antibody conjugated to a fluorophore or radioisotope, or a candidate anti-human uPAR antibody that contains an N- or C-terminal epitope tag for detection by a labeled antibody. If labels or tags are not feasible or desired, a competition binding assay can be used to determine the half-maximal inhibitory concentration (IC₅₀), the amount of unlabeled candidate anti-human uPAR antibody at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the human uPAR added in the experiment or by increasing the binding reaction volumes.

Whether an antibody of the present disclosure “competes with” a second antibody for binding to the antigen may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labeled. If the labeled antibody and the unlabeled antibody bind to separate and discrete sites on the antigen, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the antigen by about 50% or more, about 60% or more, about 70% or more, about 80% or more, about 85% or more, about 90% or more, about 95% or more, or about 99% or more. Details of procedures for carrying out such competition assays are known and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

A human uPAR polypeptide that may be used to determine whether an antibody of the present disclosure competes for binding to human uPAR with a second antibody is set forth in UniProt Q03405.

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′) 2, (scFv′) 2, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In some embodiments, the antibody is selected from an IgG, Fv, single chain antibody, scFv, Fab, F(ab′)2, F(ab′) or Fab′. The antibodies may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

An immunoglobulin light or heavy chain variable region is composed of a “framework” region (FR) interrupted by three hypervariable regions, also called “complementarity determining regions” or “CDRs”. The extent of the framework region and CDRs can be defined based on databases known in the art. See, for example, “Sequences of Proteins of Immunological Interest,” E. Kabat et al., Sequences of proteins of immunological interest, 4th ed. U.S. Dept. Health and Human Services, Public Health Services, Bethesda, MD (1987), Lefranc et al. IMGT, the international ImMunoGeneTics information System®. Nucl. Acids Res., 2005, 33: D593-D597 (www.imgt.org/textes/IMGTScientificChart/), and/or V Base at vbase.mrc-cpe.cam.ac.uk/). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Any anti-human uPAR antibody of the present disclosure may be a monoclonal antibody. As used herein, the term “monoclonal antibody” refers to an antibody composition having a homogeneous antibody population. The term is not limited by the manner in which it is made. The term encompasses whole immunoglobulin molecules, as well as Fab molecules, F(ab′)₂ fragments, Fv fragments, single chain fragment variable (scFv), fusion proteins comprising an antigen-binding portion of an antibody and a non-antibody protein, and other molecules that exhibit immunological binding properties of the parent monoclonal antibody molecule. Methods of making monoclonal antibodies are known in the art and described more fully below.

Any anti-human uPAR antibody of the present disclosure may be a recombinant or modified antibody, e.g., a chimeric, deimmunized and/or an in vitro generated antibody. The term “recombinant” or “modified” antibody as used herein is intended to include all antibodies that are prepared, expressed, created, or isolated by recombinant means, such as (i) antibodies expressed from one or more recombinant expression vectors transfected into a host cell; (ii) antibodies isolated from a recombinant, combinatorial antibody library; (iii) antibodies isolated from an animal (e.g., a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include, e.g., chimeric, deimmunized, and/or in vitro generated antibodies.

Any anti-human uPAR antibody of the present disclosure may be isolated. By “isolated” is meant that the antibody is separated from all or some of the components that accompany it in nature. “Isolated” also refers to the state of an antibody separated from all or some of the components that accompany it during manufacture, e.g., chemical synthesis, recombinant expression, culture medium, and/or the like.

Any anti-human uPAR antibody of the present disclosure may comprise an extent and/or pattern of glycosylation which is different from the extent and/or pattern of glycosylation of an antibody produced in nature, e.g., produced in an animal (e.g., produced in a human). For example, an anti-human uPAR antibody of the present disclosure may be a recombinant antibody (e.g., a monoclonal antibody) expressed from one or more recombinant expression vectors transfected into a host cell, where the expressed recombinant anti-human uPAR antibody comprises a different extent of glycosylation, a different glycosylation pattern, or both, as compared to the extent of glycosylation and/or glycosylation pattern of the antibody when produced in nature, e.g., when produced in an animal in response to immunization with a human uPAR antigen.

In some embodiments, an anti-human uPAR antibody of the present disclosure comprises a heavy chain comprising an Fc region, and the Fc region is heterologous to the V_{H} of the antibody—that is, the Fc region comprises an amino acid sequence (e.g., one or more amino acid substitutions, deletions and/or insertions), one or more post-translational modifications, and/or the like, such that an antibody comprising the combination of the Fc region and the V_{H} does not occur in nature, e.g., is different from an anti-human uPAR antibody produced in an animal in response to immunization with a human uPAR antigen.

In certain embodiments, one or more amino acid modifications may be introduced into the Fc region of an antibody provided herein, thereby generating an Fc region variant. The Fc region variant may comprise a murine Fc region sequence (e.g.: IgG1, IgG2a or IgG2b) comprising an amino acid modification (e.g., substitution) at one or more amino acid positions. The Fc region variant may comprise a human Fc region sequence (e.g., a human IgG1, IgG2, IgG3 or IgG4 Fc region) comprising an amino acid modification (e.g., substitution) at one or more amino acid positions (e.g., an IgG4 isotype including the S228P mutation).

In certain embodiments, the Fc region is mutated to increase its affinity to FcRn at pH 6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall'Acqua et al, J Immunol. 169:5171-5180 (2002)) or LS mutation M428L/N434S (Zalevsky et al, Nat Biotechnol. 28 (2): 157-159 (2010)).

The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of the antigen in the presence of a heterogeneous population of antigens (e.g., proteins and other biologics, e.g., in a sample). Thus, under designated immunoassay conditions, the specified antibodies bind to a particular human uPAR antigen and do not bind in a significant amount to other antigens present in the sample. Specific binding to an antigen under such conditions may require an antibody that is selected for its specificity for a particular antigen. For example, an anti-human uPAR antibody can specifically bind to a human uPAR antigen, and does not exhibit comparable binding (e.g., does not exhibit detectable binding) to other proteins present in a sample.

In some embodiments, an antibody of the present disclosure “specifically binds” a human uPAR antigen if it binds to or associates with the human uPAR antigen with an affinity or Kₐ (that is, an equilibrium association constant of a particular binding interaction with units of 1/M) of, for example, greater than or equal to about 10⁵ M⁻¹. In certain embodiments, the antibody binds to human uPAR with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to human uPAR with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for human uPAR can be readily determined using conventional techniques, e.g., by biolayer interferometry (BLI); competitive ELISA (enzyme-linked immunosorbent assay); equilibrium dialysis; surface plasmon resonance (SPR) technology (e.g., the BIAcore 2000 instrument, using general procedures outlined by the manufacturer); by radioimmunoassay; and/or the like.

In certain embodiments, an antibody of the present disclosure cross-reacts with a non-human animal uPAR. For example, an anti-human uPAR antibody of the present disclosure may cross-react with a non-human primate uPAR. In one non-limiting example, the non-human primate uPAR is a cynomolgus uPAR. Also by way of example, an anti-human uPAR antibody of the present disclosure may cross-react with a rodent uPAR. In some instances, the rodent uPAR is a mouse uPAR.

An antibody of the present disclosure is said to be “cross-reactive” for two different antigens or antigenic determinants (e.g., uPAR from two different species of mammal, such as human and cynomolgus monkey) if it is specific for (as defined herein) both these different antigens or antigenic determinants. In certain embodiments, an antibody binding to antigen 1 (Ag1) is “cross-reactive” to antigen 2 (Ag2) when the EC₅₀ and/or K_{D} values are in a similar range for both antigens. According to some embodiments, a monoclonal antibody binding to Ag1 is cross-reactive to Ag2 when the ratio of affinity for Ag1 to affinity for Ag2 is equal or less 10 (<10) and equal or greater than 0.1 (>0.1), which means that the affinities for Ag1 and Ag2 do not differ more than a factor of 10 (the affinities are within one order of magnitude of monovalent K_{D}), on condition that affinities are measured with the same method in the same experimental setting for both antigens. Accordingly, an antibody of the present disclosure may have a ratio of affinity for human uPAR to the affinity for cynomolgus uPAR which is equal or less 10 (<10) and equal or greater than 0.1 (>0.1), which means that the affinities for human and cynomolgus uPAR do not differ more than a factor of 10 (the affinities are within one order of magnitude of monovalent K_{D}). Such an antibody may be used, e.g., in toxicological studies performed in cynomolgus monkeys because the toxicity profile observed in cynomolgus monkeys would be relevant to anticipate potential adverse effects in humans.

An “epitope” is a site on an antigen to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Several commercial laboratories offer epitope mapping services. Epitopes bound by an antibody immunoreactive with human uPAR can reside, e.g., on the surface of human uPAR, so that such epitopes are considered human uPAR-surface accessible, solvent accessible, and/or human uPAR-surface exposed.

According to some embodiments, an anti-uPAR antibody of the present disclosure is a humanized antibody. As used herein, a humanized antibody is a recombinant polypeptide that is derived from a non-human (e.g., rabbit, rodent, or the like) antibody and has been modified to contain at least a portion of the framework and/or constant regions of a human antibody. Humanized antibodies also encompass chimeric antibodies and CDR-grafted antibodies in which various regions may be derived from different species. Chimeric antibodies may be antibodies that include a variable region from any source linked to a human constant region (e.g., a human Fc domain). Thus, in chimeric antibodies, the variable region can be non-human, and the constant region is human. CDR-grafted antibodies are antibodies that include the CDRs from a non-human “donor” antibody linked to the framework region from a human “recipient” antibody. For example, an antibody of the present disclosure in a form of an scFv may be linked to a human constant region (e.g., Fc domain) to be made into a human immunoglobulin.

In general, humanized antibodies produce a reduced immune response in a human host, as compared to a non-humanized version of the same antibody. Antibodies can be humanized using a variety of techniques including, for example, CDR-grafting, veneering or resurfacing, chain shuffling, and the like. In certain embodiments, framework substitutions are identified by modeling of the interactions of the CDR and framework residues to identify framework residues important for antigen binding and sequence comparison to identify unusual framework residues at particular positions.

Accordingly, any of the antibodies described herein may be humanized using available methods. The substitution of rabbit or mouse CDRs into a human variable domain framework can result in retention of their correct spatial orientation where, e.g., the human variable domain framework adopts the same or similar conformation to the rabbit or mouse variable framework from which the CDRs originated. This can be achieved by obtaining the human variable domains from human antibodies whose framework sequences exhibit a high degree of sequence identity with the rabbit or mouse variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Having identified the complementarity determining regions of the rabbit or mouse donor immunoglobulin and appropriate human acceptor immunoglobulins, the next step is to determine which, if any, residues from these components should be substituted to optimize the properties of the resulting humanized antibody. In general, substitution of human amino acid residues with rabbit or mouse should be minimized, because introduction of rabbit or mouse residues increases the risk of the antibody eliciting a human-anti-rabbit-antibody (HARA) or human-anti-mouse-antibody (HAMA) response in humans. Art-recognized methods of determining immune response can be performed to monitor a HARA or HAMA response in a particular patient or during clinical trials. Patients administered humanized antibodies can be given an immunogenicity assessment at the beginning and throughout the administration of said therapy. The HARA or HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HARA response in a human subject.

Certain amino acids from the human variable region framework residues are selected for substitution based on their possible influence on CDR conformation and/or binding to antigen. The unnatural juxtaposition of rabbit or murine CDR regions with human variable framework region can result in unnatural conformational restraints, which, unless corrected by substitution of certain amino acid residues, lead to loss of binding affinity. The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

When framework residues, as defined by, e.g., Kabat, constitute structural loop residues as defined by, e.g., Chothia, the amino acids present in the rabbit or mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk J M B 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody. Approaches that may be employed to humanize any of the antibodies described herein include, but are not limited to, those described in Williams, D., Matthews, D. & Jones, T. Humanising Antibodies by CDR Grafting. Antibody Engineering 319-339 (2010) doi: 10.1007/978-3-642-01144-3_21; Kuramochi, T., Igawa, T., Tsunoda, H. & Hattori, K. Humanization and simultaneous optimization of monoclonal antibody. Methods Mol. Biol. 1060, 123-37 (2014); Hwang, W. Y., Almagro, J. C., Buss, T. N., Tan, P. & Foote, J. Use of human germline genes in a CDR homology-based approach to antibody humanization. Methods 36, 35-42 (2005); Lo, B. K. Antibody humanization by CDR grafting. Methods Mol. Biol. 248, 135-59 (2004); and Lefranc, M.-P. P., Ehrenmann, F., Ginestoux, C., Giudicelli, V. & Duroux, P. Use of IMGT (®) databases and tools for antibody engineering and humanization. Methods Mol. Biol. 907, 3-37 (2012); the disclosures of which are incorporated herein by reference in their entireties for all purposes.

### Bispecific Antibodies

Also provided are bispecific antibodies. In certain embodiments, a bispecific antibody of the present disclosure comprises a first antigen-binding domain comprising a V_{H} polypeptide-V_{L} polypeptide pair of any of the anti-human uPAR antibodies of the present disclosure, including any of such antibodies described hereinabove. The bispecific antibody may include a second antigen-binding domain that specifically binds a human uPAR polypeptide bound by the first antigen-binding domain. In certain embodiments, the bispecific antibody includes a second antigen-binding domain that specifically binds an antigen other than uPAR.

Bispecific antibodies of the present disclosure include antibodies having a full-length antibody structure, and bispecific antibody fragments. “Full-length” as used herein refers to an antibody having two full-length antibody heavy chains and two full length antibody light chains. A full-length antibody heavy chain (HC) consists of well-known heavy chain variable and constant domains VH, CH1, CH2, and CH3. A full-length antibody light chain (LC) consists of well-known light chain variable and constant domains VL and CL. The full-length antibody may be lacking the C-terminal lysine in either one or both heavy chains. The term “Fab arm” refers to one heavy chain:light chain pair that specifically binds an antigen.

Full-length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in a cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US2010/0015133; US2009/0182127; US2010/028637 or US2011/0123532. In other strategies. heterodimerization may be promoted by the following substitutions (expressed as modified position in the first CH3 domain of the first heavy chain/modified position in the second CH3 domain of the second heavy chain): L351 Y_F405A_Y407V T394W, T3661_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8 (18): 3689-3696.

Approaches that may be employed to produce multispecific (e.g., bispecific) antibodies from the antibodies described herein include, but are not limited to, Ellerman, D. (2019). “Bispecific T-cell engagers: Towards understanding variables influencing the in vitro potency and tumor selectivity and their modulation to enhance their efficacy and safety.” Methods 154:102-117; Brinkmann, U. and R. E. Kontermann (2017). “The making of bispecific antibodies.” mAbs 9 (2): 182-212; and Suurs, F. V., et al. (2019). “A review of bispecific antibodies and antibody constructs in oncology and clinical challenges.” Pharmacol Ther 201:103-119; the disclosures of which are incorporated herein by reference in their entireties for all purposes.

### Fusion Proteins

Also provided are fusion proteins. In certain embodiments, a fusion protein of the present disclosure comprises a chain of any of the anti-uPAR antibodies of the present disclosure, fused to a heterologous sequence of amino acids. The heterologous sequence of amino acids may be fused to the C-terminus of the chain of the antibody or the N-terminus of the chain of the antibody. In certain embodiments, a fusion protein of the present disclosure includes a heterologous sequence at the C-terminus of the chain of the antibody and a heterologous sequence at the N-terminus of the chain of the antibody, wherein the heterologous sequences may be the same sequence or different sequences. “Heterologous” as used in the context of a nucleic acid or polypeptide generally means that the nucleic acid or polypeptide is from a different origin (e.g., molecule of different sequence, different species origin, and the like) than that with which the nucleic acid or polypeptide is associated or joined, such that the nucleic acid or polypeptide is one that is not found in nature. For example, in a fusion protein, a light chain polypeptide and a reporter polypeptide (e.g., GFP, red fluorescent protein (e.g., mCherry), luciferase, etc.) are said to be “heterologous” to one another. Similarly, a CDR from a mouse antibody and a constant region from a human antibody are “heterologous” to one another.

The chain of the anti-human uPAR antibody may be fused to any heterologous sequence of interest. Heterologous sequences of interest include, but are not limited to, an albumin, a transferrin, XTEN, a homo-amino acid polymer, a proline-alanine-serine polymer, an elastin-like peptide, or any combination thereof. In certain aspects, the heterologous polypeptide increases the stability and/or serum half-life of the antibody upon its administration to an individual in need thereof, as compared to the same antibody which is not fused to the heterologous sequence.

In certain embodiments, a fusion protein of the present disclosure comprises a single chain antibody, e.g., a single chain antibody (e.g., scFv) comprising a V_{H} polypeptide-V_{L} polypeptide pair of any of the anti-human uPAR antibodies of the present disclosure, including any of such antibodies described hereinabove.

According to some embodiments, when the fusion protein comprises a single chain antibody (e.g., any of the single chain antibodies of the present disclosure, including any of the scFvs described herein), the fusion protein is a chimeric antigen receptor (CAR) comprising the single chain antibody, a transmembrane domain, and an intracellular signaling domain.

A CAR of the present disclosure may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a serine-glycine linker, such as a serine-glycine linker that includes the amino acid sequence GGGGGGGGSGGGGS (G₄S)₃ (SEQ ID NO:54). In certain aspects, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, the antigen binding domain of the CAR is followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8a and CD4, which may be wild-type hinge regions from these molecules or variants thereof. In certain aspects, the hinge domain includes a CD8a hinge region. In some embodiments, the hinge is a PD-1 hinge or CD152 hinge.

The “transmembrane domain” (Tm domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD33, CD3γ, CD30, CD4, CD5, CD8a, CD9, CD16, CD22, CD27, CD28, CD33, CD37, CD45, CD64, CD80, CD86, CD134, CD137, CD152, CD154, or PD-1.

In one embodiment, a CAR includes a Tm domain derived from CD8a. In certain aspects, a CAR includes a Tm domain derived from CD8a and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion of a protein which transduces the effector function signal and that directs the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a CAR of the present disclosure may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR of the present disclosure include those derived from FcRγ, FcRβ, CD3γ, CD3δ, CD3ε, CD3ζ, CD22, CD79α, CD79β, and CD66δ. In certain embodiments, a CAR includes a CD3ζ primary signaling domain and one or more costimulatory signaling domains. The intracellular primary signaling and costimulatory signaling domains are operably linked to the carboxyl terminus of the transmembrane domain.

In some embodiments, the CAR includes one or more costimulatory signaling domains to enhance the efficacy and expansion of immune effector cells (e.g., T cells) expressing the CAR. As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the CAR includes one or more costimulatory signaling domains selected from the group consisting of 4-1BB (CD137), CD28, and CD134, and a CD3Z primary signaling domain.

A CAR of the present disclosure may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains; and/or the like. Non-limiting examples of such domains that may be included in a CAR of the present disclosure include those provided in Table 6 below. As will be appreciated by one of ordinary skill in the art, the amino acid sequence of one or more of the domains indicated in Table 6 (e.g., linker, hinge, transmembrane, co-stimulatory, signaling, ribosomal skip element; restriction enzyme sequence; reporter protein etc.) may be modified as desired, e.g., for improved functionality, etc. of the CAR.

In certain aspects, a CAR of the present disclosure includes a single chain antibody (e.g., any of the scFvs of the present disclosure) that binds to human uPAR; a transmembrane domain from a polypeptide selected from the group consisting of: CD4, CD8a, CD154, and PD-1; one or more intracellular costimulatory signaling domains from a polypeptide selected from the group consisting of: 4-1BB (CD137), CD28, and CD134; and an intracellular signaling domain from a polypeptide selected from the group consisting of: FcRγ, FcRβ, CD3γ, CD3δ, CD3ε, CD3ζ, CD22, CD79α, CD79β, and CD66δ. Such a CAR may further include a spacer domain between the antigen-binding portion and the transmembrane domain, e.g., a CD8 alpha hinge.

According to some embodiments, provided are CARs that comprise—from N-terminus to C-terminus—a variable heavy chain (VA) polypeptide of an antibody described herein, a linker, the variable light chain (V_{L}) of the antibody, a CD8 hinge region (which in some embodiments is an extended CD8 hinge region), a CD8 transmembrane domain, a 4-1BB costimulatory domain, and a CD3 signaling domain. According to certain embodiments, provided are CARs that comprise—from N-terminus to C-terminus—a variable light chain (V_{L}) polypeptide of an antibody described herein, a linker, the variable heavy chain (V_{H}) of the antibody, a CD8 hinge region (which in some embodiments is an extended CD8 hinge region), a CD8 transmembrane domain, a 4-1BB costimulatory domain, and a CD3ζ signaling domain. In certain embodiments, provided are CARs that comprise—from N-terminus to C-terminus—a variable heavy chain (V_{H}) polypeptide of an antibody described herein, a linker, the variable light chain (V_{L}) of the antibody, a CD28 hinge region, a CD28 transmembrane domain, a 4-1BB costimulatory domain, and a CD3ζ signaling domain. According to some embodiments, provided are CARs that comprise—from N-terminus to C-terminus—a variable light chain (V_{L}) polypeptide of an antibody described herein, a linker, the variable heavy chain (V_{H}) of the antibody, a CD28 hinge region, a CD28 transmembrane domain, a 4-1BB costimulatory domain, and a CD3ζ signaling domain. Any of the CARs of the present disclosure may include a domain N-terminal to the V_{H} polypeptide. For example, a leader sequence (e.g., a GM-CSFR leader sequence) may be present at the N-terminus of a CAR of the present disclosure.

A CAR of the present disclosure may include one or more additional domains as desired. Non-limiting examples of such additional domains include a ribosomal skip element, an enzymatic domain (e.g., a domain having nuclease activity, e.g., restriction endonuclease activity), a domain that enables detection of the CAR (e.g., a reporter protein domain (e.g., a fluorescent protein (e.g., eGFP, mCherry, or the like), a luminescent protein, and/or the like)), etc. For example, in certain embodiments, provided are CARs that comprise a ribosomal skip element, a restriction enzyme domain, and/or a reporter protein domain.

According to some embodiments, a CAR of the present disclosure is provided by a single polypeptide. In certain embodiments, a CAR of the present disclosure is provided by two or more polypeptides. When the CAR is provided by two or more polypeptides, the CAR may be provided in any useful multi-polypeptide format, including universal CAR formats such as biotin-binding immune receptor (BBIR) format (see, e.g., Urbanska K, Powell D J. Development of a novel universal immune receptor for antigen targeting to infinity and beyond. Oncoimmunology. 2012; 1 (5): 777-779. doi: 10.4161/onci.19730, and Urbanska K, Lanitis E, Poussin M, et al. A universal strategy for adoptive immunotherapy of cancer through use of a novel T cell antigen receptor. 2013; 72 (7): 1844-1852. doi: 10.1158/0008-5472.CAN-11-3890.A); a switchable CAR format with peptide NeoEpitope (PNE) (see, e.g., Kim et al. (2015) J Am Chem Soc. 2015; 137 (8): 2832-2835; Ma et al. (2016) Proc Natl Acad Sci 113 (4):E450-8; Rodgers et al. (2016) Proc Natl Acad Sci. 113 (4): E459-E468; Viaud et al. (2018) Proc Natl Acad Sci 115 (46):E10898-E10906); a SUPRA CAR format with leucine zippers (see, e.g., Cho et al. (2108) Cell 173 (6): 1426-1438.e11); a CAR-T Adapter Molecule (CAM)-based format with FITC-folic acid (see, e.g., Lee et al. (2019) Cancer Res. 79 (2): 387-396; and Lu et al. (2019) Front Oncol. 9:151); anti-FITC-folic acid adaptor format (see, e.g., Chu et al. (2018) Biosci Trends. 12 (3): 298-308); anti-FITC antibody adaptor CAR format (see, e.g., Tamada et al. (2012) Clin Cancer Res. 18 (23): 6436-6445); Fc-targeting (e.g., anti-CD16) CAR+anti-tumor antibody format (see, e.g., Kudo et al. (2014) Cancer Res. 74 (1): 93-103); and the like.

### Conjugates

The present disclosure also provides conjugates. According to some embodiments, a conjugate of the present disclosure comprises any of the antibodies or fusion proteins of the present disclosure, and an agent conjugated to the antibody or fusion protein. The term “conjugated” generally refers to a chemical linkage, either covalent or non-covalent, usually covalent, that proximally associates one molecule of interest with a second molecule of interest. In certain embodiments, the agent conjugated to the antibody or fusion protein is a chemotherapeutic agent, a toxin, a radiation-sensitizing agent, a radioactive isotope (e.g., a therapeutic radioactive isotope), a detectable label, or a half-life extending moiety.

According to some embodiments, the agent is a therapeutic agent, e.g., a chemotherapeutic agent. As used herein, a “therapeutic agent” is a physiologically or pharmacologically active substance that can produce a desired biological effect in a targeted site in an animal, such as a mammal or in a human. The therapeutic agent may be any inorganic or organic compound. Examples include, without limitation, peptides, proteins, nucleic acids (including siRNA, miRNA and DNA), polymers, and small molecules. A therapeutic agent may decrease, suppress, attenuate, diminish, arrest, or stabilize the development or progression of disease, disorder, or cell growth in an animal such as a mammal or human. Therapeutic agents of interest include agents capable of affecting the function of a cell/tissue to which the conjugate binds via specific binding of the antibody portion of the conjugate to the antigen. When the function of the cell/tissue is pathological, an agent that reduces the function of the cell/tissue may be employed. In certain aspects, a conjugate of the present disclosure includes an agent that reduces the function of a target cell/tissue by inhibiting cell proliferation and/or killing the cell/tissue. Such agents may vary and include cytostatic agents and cytotoxic agents, e.g., an agent capable of killing a target cell tissue with or without being internalized into a target cell.

In certain embodiments, the therapeutic agent is a cytotoxic agent selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

According to some embodiments, the agent is a toxin, such as a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

In certain embodiments, the agent is a radiation-sensitizing agent. As used herein, a “radiation-sensitizing agent” is an agent that enhances the ability of radiation to kill tumor cells. Non-limiting examples of radiation-sensitizing agents that may be conjugated to the antibody or fusion protein include cisplatin, 5-fluorouracil (5-FU), AZD7762, selumetinib, and the like.

In certain embodiments, the agent is a radioisotope, e.g., useful for therapy and/or detection (e.g., imaging). Non-limiting examples of radioisotopes that may be conjugated to the antibody or fusion protein include but are not limited to ²²⁵Ac, ¹¹¹Ag, ¹¹⁴Ag, ⁷¹As, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ¹³C, ⁵⁵Co, ⁶²Cu, ⁶⁴Cu, ⁶⁷Cu, ¹⁶⁵Dy, ¹⁶⁶Dy, ¹⁶⁹Er, ¹⁸F, ¹⁹F, ⁵²Fe, ⁵⁹Fe, ⁶⁶Ga, ⁶⁷Ga, ⁶⁸Ga, ⁷²Ga, ¹⁵⁴⁻¹⁵⁸Gd, ¹⁵⁷Gd, ¹⁵⁹Gd, ¹⁶⁶Ho, ¹²⁰I, ¹²¹I, ¹²³I, ¹²⁴I, ¹²⁵I, ¹³¹I, ¹¹⁰In, ¹¹¹In, ^{113m}In, ¹⁹⁴Ir, ^{81m}Kr, ¹⁷⁷Lu, ⁵¹Mn, ⁵²Mn, ⁹⁹Mo, ¹³N, ¹⁵N, ¹⁵O, ¹⁷O, ³²P, ³³P, ²¹¹Pb, ²¹²Pb, ¹⁰⁹Pd, ¹⁴⁹Pm, ¹⁵¹Pm, ¹⁴²Pr, ¹⁴³Pr, ¹⁹¹PT, ^{193m}PT, ^{195m}Pt, ²²³Ra, ¹⁴²Rb, ¹⁸⁶Re, ¹⁸⁸Re, ¹⁸⁹Re, ¹⁰⁵Rh, ⁴⁷Sc, ⁷⁵Se, ¹⁵³Sm, ^{117m}Sn, ¹²¹Sn, ⁸³Sr, ⁸⁹Sr, ¹⁶¹Tb, ⁹⁴Tc, ⁹⁹Tc, ^{99m}Tc, ²²⁷Th, ²⁰¹Tl, ¹⁷²Tm, ¹²⁷Te, ⁹⁰Y, ¹⁶⁹Yb, ¹⁷⁵Yb, ¹³³X, and ⁸⁹Zr.

In certain embodiments, a radioisotope is conjugated to the antibody or fusion protein via a chelator, for example, a bifunctional chelator. A bifunctional chelator may contain a metal chelating moiety that binds the radioisotope in a stable coordination complex and a reactive functional group that is covalently linked to a targeting moiety, such as any of the antibodies or fusion proteins of the present disclosure, so that the radioisotope may be properly directed to the desirable molecular target in vivo. Examples of bifunctional chelators that may be employed to conjugate an antibody or fusion protein of the present disclosure to a radioisotope include those described in Price & Orvig (2014) Chem. Soc. Rev. 43:260; and Brechbiel (2008) Q J Nucl Med Mol Imaging 52 (2): 166-173.

According to some embodiments, the radioisotope is a therapeutic radioisotope. In certain embodiments, the radioisotope is an alpha emitting radioisotope. e.g., ²²⁵Ac, ²¹¹At, ²¹²Bi/²¹²Pb, ²¹³Bi, ²²³Ra, or ²²⁷Th. In other embodiments, the radioisotope is a beta minus emitting radioisotope, e.g., ³²P, ³³P, ⁶⁷Cu, ⁹⁰Y, ¹³¹I or ¹⁷⁷Lu.

According to some embodiments, the agent is a labeling agent. By “labeling agent” (or “detectable label”) is meant the agent detectably labels the antibody or fusion protein, such that the antibody or fusion protein may be detected in an application of interest (e.g., in vitro and/or in vivo research and/or clinical applications). Detectable labels of interest include radioisotopes (e.g., gamma or positron emitters), enzymes that generate a detectable product (e.g., horseradish peroxidase, alkaline phosphatase, luciferase, etc.), fluorescent proteins, paramagnetic atoms, and the like. In certain aspects, the antibody or fusion protein is conjugated to a specific binding partner of detectable label, e.g., conjugated to biotin such that detection may occur via a detectable label that includes avidin/streptavidin.

In certain embodiments, the agent is a labeling agent that finds use in in vivo imaging, such as near-infrared (NIR) optical imaging, single-photon emission computed tomography (SPECT)±CT imaging, positron emission tomography (PET)±CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 800CW Fluors. According to some embodiments, the labeling agent is an in vivo imaging agent that finds use in SPECT imaging applications, non-limiting examples of which include ^{99m}Tc, ¹¹¹In, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

For half-life extension, the antibodies and fusion proteins of the present disclosure may be conjugated to an agent that provides for an improved pharmacokinetic profile (e.g., by PEGylation, hyperglycosylation, and the like). Modifications that can enhance serum half-life are of interest. A subject antibody or fusion protein may be “PEGylated”, as containing one or more poly(ethylene glycol) (PEG) moieties. Methods and reagents suitable for PEGylation of a protein are well known in the art and may be found, e.g., in U.S. Pat. No. 5,849,860. PEG suitable for conjugation to a protein is generally soluble in water at room temperature and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody or fusion protein can be linear. The PEG conjugated to the subject antibody or fusion protein may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Where the subject antibody or fusion protein is to be isolated from a source, the antibody or fusion protein may be conjugated to one or more moieties that facilitate purification, such as members of specific binding pairs, e.g., biotin (member of biotin-avidin specific binding pair), a lectin, and the like. The antibody can also be bound to (e.g., immobilized onto) a solid support, including, but not limited to, polystyrene plates or beads, magnetic beads, test strips, membranes, and the like.

Where the antibodies or fusion proteins are to be detected in an assay, the antibodies or fusion proteins may contain a detectable label, e.g., a radioisotope (e.g., 89Zr; 111 In, and the like), an enzyme which generates a detectable product (e.g., luciferase, β-galactosidase, horse radish peroxidase, alkaline phosphatase, and the like), a fluorescent protein, a chromogenic protein, dye (e.g., fluorescein isothiocyanate, rhodamine, phycoerythrin, and the like); fluorescence emitting metals, e.g., 152Eu, or others of the lanthanide series, attached to the protein through metal chelating groups such as EDTA; chemiluminescent compounds, e.g., luminol, isoluminol, acridinium salts, and the like; bioluminescent compounds, e.g., luciferin; fluorescent proteins; and the like. Indirect labels include antibodies specific for a subject protein, wherein the antibody may be detected via a secondary antibody; and members of specific binding pairs, e.g., biotin-avidin, and the like.

Any of the above agents may be conjugated to the antibody or fusion protein via a linker. If present, the linker molecule(s) may be of sufficient length to permit the antibody or fusion protein and the linked agent to allow some flexible movement between the antibody or fusion protein and the linked agent. Linker molecules may be, e.g., about 6-50 atoms long. Linker molecules may also be, e.g., aryl acetylene, ethylene glycol oligomers containing 2-10 monomer units, diamines, diacids, amino acids, or combinations thereof.

Where the linkers are peptides, the linkers can be of any suitable length, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids in length.

Flexible linkers include glycine polymers (G) n, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of an antibody or fusion protein conjugated to any agents described above can include linkers that are all or partially flexible, such that the linker can include a flexible linker as well as one or more portions that confer a less flexible structure.

According to some embodiments, the antibody or fusion protein is conjugated to the agent via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the antibody is conjugated to the agent via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9 (1): 33-46; and elsewhere.

Numerous strategies are available for linking agents to an antibody or fusion protein directly, or indirectly via a linker. For example, the agent may be derivatized by covalently attaching a linker to the agent, where the linker has a functional group capable of reacting with a “chemical handle” on the antibody or fusion protein. The functional group on the linker may vary and may be selected based on compatibility with the chemical handle on the antibody or fusion protein. According to one embodiment, the chemical handle on the antibody or fusion protein is provided by incorporation of an unnatural amino acid having the chemical handle into the antibody or fusion protein. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into an antibody of a conjugate of the present disclosure, which unnatural amino acid may be selected to provide a functional group of interest, are known and described in, e.g., Maza et al. (2015) Bioconjug. Chem. 26 (9): 1884-9; Patterson et al. (2014) ACS Chem. Biol. 9:592-605; Adumeau et al. (2016) Mol. Imaging Biol. (2): 153-65; and elsewhere. An unnatural amino acid may be incorporated into an antibody or fusion protein via chemical synthesis or recombinant approaches, e.g., using a suitable orthogonal amino acyl tRNA synthetase-tRNA pair for incorporation of the unnatural amino acid during translation of the antibody or fusion protein in a host cell.

The functional group of an unnatural amino acid present in the antibody or fusion protein may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the antibody or fusion protein and the linker portion of a linker-agent moiety may include an alkyne functional group, such that the antibody or fusion protein and linker-agent moiety are covalently conjugated via azide-alkyne cycloaddition. Conjugation may be carried out using, e.g., a copper-catalyzed azide-alkyne cycloaddition reaction.

In certain embodiments, the chemical handle on the antibody or fusion protein does not involve an unnatural amino acid. An antibody containing no unnatural amino acids may be conjugated to the agent by utilizing, e.g., nucleophilic functional groups of the antibody or fusion protein (such as the N-terminal amine or the primary amine of lysine, or any other nucleophilic amino acid residue) as a nucleophile in a substitution reaction with a moiety bearing a reactive leaving group or other electrophilic group. An example would be to prepare an agent-linker moiety bearing an N-hydroxysuccinimidyl (NHS) ester and allow it to react with the antibody or fusion protein under aqueous conditions at elevated pH (˜10) or in polar organic solvents such as DMSO with an added non-nucleophilic base, such as N,N-diisopropylethylamine.

It will be appreciated that the particular approach for attaching a linker, agent and/or antibody or fusion protein to each other may vary depending upon the particular linker, agent and/or antibody or fusion protein and functional groups selected and employed for conjugating the various components to each other.

### Methods of Producing Antibodies

Using the information provided herein, the anti-uPAR antibodies and fusion proteins of the present disclosure may be prepared using standard techniques well known to those of skill in the art. For example, a nucleic acid sequence(s) encoding the amino acid sequence of an antibody or fusion protein of the present disclosure can be used to express the antibodies or fusion proteins. The polypeptide sequences provided herein (see, e.g., Table 1) can be used to determine appropriate nucleic acid sequences encoding the antibodies or fusion proteins and the nucleic acids sequences then used to express one or more antibodies or fusion proteins specific for human uPAR. The nucleic acid sequence(s) can be optimized to reflect particular codon “preferences” for various expression systems according to standard methods well known to those of skill in the art. Using the sequence information provided, the nucleic acids may be synthesized according to a number of standard methods known to those of skill in the art.

Once a nucleic acid(s) encoding a subject antibody is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies and fusion proteins of the present disclosure can be achieved by operably linking a nucleic acid encoding the antibody or fusion protein to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject antibody can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody can be further purified by dialysis and other methods normally employed in protein purification methods. In one embodiment, the antibody may be isolated using metal chelate chromatography methods. Antibodies of the present disclosure may contain modifications to facilitate isolation, as discussed above.

The antibodies may be prepared in substantially pure or isolated form (e.g., free from other polypeptides). The protein can be present in a composition that is enriched for the polypeptide relative to other components that may be present (e.g., other polypeptides or other host cell components). Purified antibodies may be provided such that the antibody is present in a composition that is substantially free of other expressed proteins, e.g., less than 90%, usually less than 60% and more usually less than 50% of the composition is made up of other expressed proteins.

The antibodies produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the antibodies may be operably linked to a secretion signal sequence such as pelB so that the antibodies are secreted into the periplasm in correctly-folded form.

The present disclosure also provides cells that produce the antibodies of the present disclosure, where suitable cells include eukaryotic cells, e.g., mammalian cells. The cells can be a hybrid cell or “hybridoma” that is capable of reproducing antibodies in vitro (e.g. monoclonal antibodies, such as IgG). For example, the present disclosure provides a recombinant host cell (also referred to herein as a “genetically modified host cell”) that is genetically modified with one or more nucleic acids comprising a nucleotide sequence encoding a heavy and/or light chain of an antibody of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia), insect or mammalian expression system, for example. One example of a suitable technique uses a bacteriophage lambda vector system having a leader sequence that causes the expressed antibody (e.g. Fab or scFv) to migrate to the periplasmic space (between the bacterial cell membrane and the cell wall) or to be secreted. One can rapidly generate great numbers of functional fragments (e.g. Fab or scFv) for those which bind the antigen of interest.

Antibodies that specifically bind human uPAR can be prepared using a wide variety of techniques known in the art including the use of hybridoma, recombinant, phage display technologies, Selected Lymphocyte Antibody Method (SLAM), or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds human uPAR can be selected or identified with human uPAR, e.g., using labeled human uPAR bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene Ill or gene Vill protein. The production of high affinity human antibodies by chain shuffling is known, as are combinatorial infection and in vivo recombination as a strategy for constructing large phage libraries. In another embodiment, ribosomal display can be used to replace bacteriophage as the display platform. Cell surface libraries may be screened for antibodies. Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

### Nucleic Acids, Expression Vectors and Cells

In view of the section above regarding methods of producing the antibodies and fusion proteins of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an antibody or fusion protein of the present disclosure, including any of the anti-human uPAR antibodies of the present disclosure, e.g., any of such antibodies described hereinabove. According to some embodiments, the antibody is a single chain antibody (e.g., an scFv), and the nucleic acid encodes the single chain antibody.

According to some embodiments, provided is a nucleic acid that encodes a CAR of the present disclosure, e.g., a CAR comprising: a single chain antibody comprising a V_{H} polypeptide and a V_{L} polypeptide of an anti-human uPAR antibody of the present disclosure; a transmembrane domain; and an intracellular signaling domain. Examples of such single chain antibodies, transmembrane domains, and intracellular signaling domains are described in detail above.

Also provided are expression vectors comprising any of the nucleic acids of the present disclosure. Expression of natural or synthetic nucleic acids encoding the antibodies and fusion proteins of the present disclosure can be achieved by operably linking a nucleic acid encoding the antibody or fusion protein to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Cells that comprise any of the nucleic acids and/or expression vectors of the present disclosure are also provided. According to some embodiments, a cell of the present disclosure includes a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such as cell comprises a first expression vector comprising the first nucleic acid, and a second expression vector comprising the second nucleic acid.

Also provided are methods of making an antibody or fusion protein of the present disclosure, the methods including culturing a cell of the present disclosure under conditions suitable for the cell to express the antibody or fusion protein, wherein the antibody or fusion protein is produced. The conditions for culturing the cell such that the antibody or fusion protein is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%).

### Compositions

As summarized above, the present disclosure also provides compositions. According to some embodiments, a composition of the present disclosure includes an antibody, fusion protein, or conjugate of the present disclosure. For example, the antibody, fusion protein, or conjugate may be any of the antibodies, fusion proteins, or conjugates described in the Antibodies section hereinabove, which descriptions are incorporated but not reiterated herein for purposes of brevity.

In certain aspects, a composition of the present disclosure includes the antibody, fusion protein, or conjugate present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure includes an anti-human uPAR antibody of the present disclosure (or conjugate or fusion protein comprising same), and a pharmaceutically acceptable carrier.

The antibodies, fusion proteins, or conjugates can be incorporated into a variety of formulations for therapeutic administration. More particularly, the antibodies, fusion proteins, or conjugates can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the antibodies, fusion proteins, or conjugates for administration to an individual (e.g., suitable for human administration) are generally sterile and may further be free of detectable pyrogens or other contaminants contraindicated for administration to a patient according to a selected route of administration.

In pharmaceutical dosage forms, the antibodies, fusion proteins, or conjugates can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the antibodies, fusion proteins, or conjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The antibodies, fusion proteins, or conjugates can be formulated for parenteral (e.g., intravenous, intra-arterial, intraosseous, intramuscular, intracerebral, intracerebroventricular, intrathecal, subcutaneous, etc.) administration. In certain aspects, the antibodies, fusion proteins, or conjugates are formulated for injection by dissolving, suspending or emulsifying the antibodies, fusion proteins, or conjugates in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the antibodies, fusion proteins, or conjugates may be prepared by mixing the antibodies, fusion proteins, or conjugates having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the antibodies, fusion proteins, or conjugates may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/V.

A lyoprotectant may also be added in order to protect the antibody, fusion protein, or conjugate against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the antibody, fusion protein, or conjugate, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Aspects of the present disclosure further include kits. In certain embodiments, the kits find use in practicing the methods of the present disclosure, including but not limited to, methods of treating a condition associated with uPAR expression and/or activity in a subject in need thereof.

Accordingly, in certain embodiments, a kit of the present disclosure comprises any of the pharmaceutical compositions of the present disclosure, and instructions for administering the pharmaceutical composition to an individual in need thereof. The pharmaceutical composition included in the kit may include any of the antibodies, fusion proteins, and/or conjugates of the present disclosure, e.g., any of the antibodies, fusion proteins, and/or conjugates described hereinabove. As will be appreciated, the kits of the present disclosure may include any of the agents and features described above in the sections relating to the subject antibodies, fusion proteins, conjugates and compositions, which are not reiterated herein for purposes of brevity.

The kits of the present disclosure may include a quantity of the compositions, present in unit dosages, e.g., ampoules, or a multi-dosage format. As such, in certain embodiments, the kits may include one or more (e.g., two or more) unit dosages (e.g., ampoules) of a composition that includes an antibody, fusion protein, and/or conjugate of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular antibody, fusion protein, and/or conjugate employed, the effect to be achieved, and the pharmacodynamics associated with the antibody, fusion protein, and/or conjugate, in the individual. In yet other embodiments, the kits may include a single multi dosage amount of the composition.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods of Use

Aspects of the present disclosure further include methods of using the antibodies, fusion proteins (e.g., CARs), and conjugates of the present disclosure. The methods are useful in a variety of contexts, including in vitro and/or in vivo research and/or clinical applications.

In certain embodiments, provided are methods of treating a condition associated with uPAR expression and/or activity in a subject in need thereof, the methods comprising administering an effective amount of a composition comprising an antibody, fusion protein (e.g., CAR), or conjugate of the present disclosure to the subject.

According to some embodiments, the condition associated with uPAR expression and/or activity is cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. According to some embodiments, the cancer is characterized by cancer cells that express uPAR on the surface thereof. In certain embodiments, the cancer comprises a solid tumor. According to some embodiments, the solid tumor is a carcinoma, lymphoma, blastoma, or sarcoma. In some embodiments, when the cancer comprises a solid tumor, the cancer is characterized by stromal cells in the tumor microenvironment that express uPAR on the surface thereof.

Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, breast cancer, squamous non-small cell lung cancer, rhabdomyosarcoma, metastatic renal cell carcinoma, basal cell carcinoma (basal cell epithelioma), and gliosarcoma. According to some embodiments, the subject has breast cancer, lung cancer, bladder cancer, ovarian cancer, prostate cancer, liver cancer, colon cancer, pancreatic cancer, gastric cancer, glioma, or any combination thereof.

In certain embodiments, the cancer comprises a hematological malignancy. Non-limiting examples of hematological malignancies include leukemia, a lymphoma, and multiple myeloma.

According to some embodiments, provided are methods of inhibiting tumor invasion, tumor metastasis, degradation of extracellular matrix (ECM), tumor angiogenesis, tumor cell proliferation, or any combination thereof, in a subject having cancer, the method comprising administering an effective amount of a composition comprising an antibody, fusion protein (e.g., CAR), or conjugate of the present disclosure to the subject.

The antibodies, fusion proteins and conjugates of the present disclosure may be administered via any suitable route of administration, e.g., oral (e.g., in tablet form, capsule form, liquid form, or the like), parenteral (e.g., by intravenous, intra-arterial, subcutaneous, intramuscular, or epidural injection), topical, intra-nasal, intra-tumoral administration, or the like.

The antibodies, fusion proteins and conjugates of the present disclosure may be administered in a composition in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer, as compared to a control. With respect to cancer, in some embodiments, the therapeutically effective amount is sufficient to slow the growth of a tumor, reduce the size of a tumor, and/or the like. An effective amount can be administered in one or more administrations.

As described above, aspects of the present disclosure include methods for treating a cancer of an individual. By treatment is meant at least an amelioration of one or more symptoms associated with the cancer of the individual, where amelioration is used in a broad sense to refer to at least a reduction in the magnitude of a parameter, e.g. symptom, associated with the cancer being treated. As such, treatment also includes situations where the cancer, or at least one or more symptoms associated therewith, are completely inhibited, e.g., prevented from happening, or stopped, e.g., terminated, such that the individual no longer suffers from the cancer, or at least the symptoms that characterize the cancer.

An antibody, fusion protein, or conjugate of the present disclosure may be administered to the individual alone or in combination with a second agent. Second agents of interest include, but are not limited to, agents approved by the United States Food and Drug Administration and/or the European Medicines Agency (EMA) for use in treating cancer. In some embodiments, the second agent is an immune checkpoint inhibitor. Immune checkpoint inhibitors of interest include, but are not limited to, a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When an antibody, fusion protein, or conjugate of the present disclosure is administered with a second agent, the antibody, fusion protein, or conjugate and the second agent may be administered to the individual according to any suitable administration regimen. According to certain embodiments, the antibody, fusion protein, or conjugate and the second agent are administered according to a dosing regimen approved for individual use. In some embodiments, the administration of the antibody, fusion protein, or conjugate permits the second agent to be administered according to a dosing regimen that involves one or more lower and/or less frequent doses, and/or a reduced number of cycles as compared with that utilized when the second agent is administered without administration of the antibody, fusion protein, or conjugate. In certain aspects, the administration of the second agent permits the antibody, fusion protein, or conjugate to be administered according to a dosing regimen that involves one or more lower and/or less frequent doses, and/or a reduced number of cycles as compared with that utilized when the antibody, fusion protein, or conjugate is administered without administration of the second agent.

In some embodiments, one or more doses of the antibody, fusion protein, or conjugate and the second agent are administered concurrently to the individual. By “concurrently” is meant the antibody, fusion protein, or conjugate and the second agent are either present in the same pharmaceutical composition, or the antibody, fusion protein, or conjugate and the second agent are administered as separate pharmaceutical compositions within 1 hour or less, 30 minutes or less, or 15 minutes or less.

In some embodiments, one or more doses of the antibody, fusion protein, or conjugate and the second agent are administered sequentially to the individual.

In some embodiments, the antibody, fusion protein, or conjugate and the second agent are administered to the individual in different compositions and/or at different times. For example, the antibody, fusion protein, or conjugate may be administered prior to administration of the second agent, e.g., in a particular cycle. Alternatively, the second agent may be administered prior to administration of the antibody, fusion protein, or conjugate, e.g., in a particular cycle. The second agent to be administered may be administered a period of time that starts at least 1 hour, 3 hours, 6 hours, 12 hours, 24 hours, 48 hours, 72 hours, or up to 5 days or more after the administration of the first agent to be administered.

In one example, the second agent is administered to the individual for a desirable period of time prior to administration of the antibody, fusion protein, or conjugate. In certain aspects, such a regimen “primes” the cancer cells to potentiate the anti-cancer effect of the antibody, fusion protein, or conjugate. Such a period of time separating a step of administering the second agent from a step of administering the antibody, fusion protein, or conjugate is of sufficient length to permit priming of the cancer cells, desirably so that the anti-cancer effect of the antibody, fusion protein, or conjugate is increased.

In some embodiments, administration of one agent is specifically timed relative to administration of the other agent. For example, in some embodiments, the antibody, fusion protein, or conjugate is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest.

In some embodiments, the antibody, fusion protein, or conjugate and the second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of a second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between at least one pair of doses of the first agent. In some embodiments, different doses of the same agent are separated by a common interval of time; in some embodiments, the interval of time between different doses of the same agent varies. In certain aspects, different doses of the antibody, fusion protein, or conjugate and the second agent are separated from one another by a common interval of time; in some embodiments, different doses of the different agents are separated from one another by different intervals of time.

One exemplary protocol for interdigitating two intermittent, cycled dosing regimens may include: (a) a first dosing period during which a therapeutically effective amount the antibody, fusion protein, or conjugate is administered to the individual; (b) a first resting period; (c) a second dosing period during which a therapeutically effective amount of the second agent is administered to the individual; and (d) a second resting period. A second exemplary protocol for interdigitating two intermittent, cycled dosing regimens may include: (a) a first dosing period during which a therapeutically effective amount the second agent is administered to the individual; (b) a first resting period; (c) a second dosing period during which a therapeutically effective amount of the antibody, fusion protein, or conjugate is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the therapy is administered; properties (e.g., pharmacokinetic properties) of the antibody, fusion protein, or conjugate, and/or one or more features of the patient's response to therapy with the antibody, fusion protein, or conjugate. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below a pre-determined level, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The antibody, fusion protein, or conjugate and the second agent may be administered together or independently via any suitable route of administration. The antibody, fusion protein, or conjugate and the second agent may be administered via a route of administration independently selected from oral, parenteral (e.g., by intravenous, intra-arterial, subcutaneous, intramuscular, or epidural injection), topical, intra-nasal, intra-tumoral administration, or the like. According to certain embodiments, antibody, fusion protein, or conjugate and the second agent are both administered orally or parenterally (e.g., in tablet form, capsule form, liquid form, or the like) either concurrently (in the same pharmaceutical composition or separate pharmaceutical compositions) or sequentially.

The following examples are offered by way of illustration and not by way of limitation.

### EXPERIMENTAL

Based on the important role of uPAR as an anti-cancer target, therapeutic antibodies targeting uPAR for anti-cancer therapy have been developed. Fully human rAbs, 2G10 and 3C6 (27), were identified from a human naïve Fab library with phage display technology and shown to be effective against human TNBC cells in xenograft models (17). The antitumor efficacy of the antibodies was increased with either therapeutic radionuclide or as antibody-drug conjugates (ADC) (17,24,27). However, they lacked cross-reactivity, limiting their advancement as clinical candidates. In the development process of a therapeutic antibody, the precise prediction of human pharmacokinetics, toxicity, and potency for an antibody before first in-human studies is fundamental to its development as an effective biotherapeutic entity (28). The cynomolgus monkeys (cyno) are genetically similar to human compared to other species and are the most relevant non-human primate model for conducting pre-clinical studies in the development of antibody drugs (29).

Described herein is the establishment of an accelerated discovery approach for the development of novel human and cyno cross-reactive rAbs by using a microfluidic platform and optoelectro tweezers to screen uPAR-primed mouse B lymphocytes. Unique cross-reactive rAbs were shown to exhibit antibody-dependent cellular cytotoxicity (ADCC), ADC cytotoxicity, and inhibitory effects on cell adhesion against human breast cancer cells. Furthermore, lead antibodies showed their therapeutic efficacy in reducing tumor growth in an orthotopic animal model of human breast cancer, providing promising rAb candidates. Finally, a binding model of lead antibodies is provided showing their binding epitopes that lead to unique activities against uPAR.

### Example 1—High-Throughput B-Cell Screening for Human and Cyno uPAR Cross-Reactive Antibodies

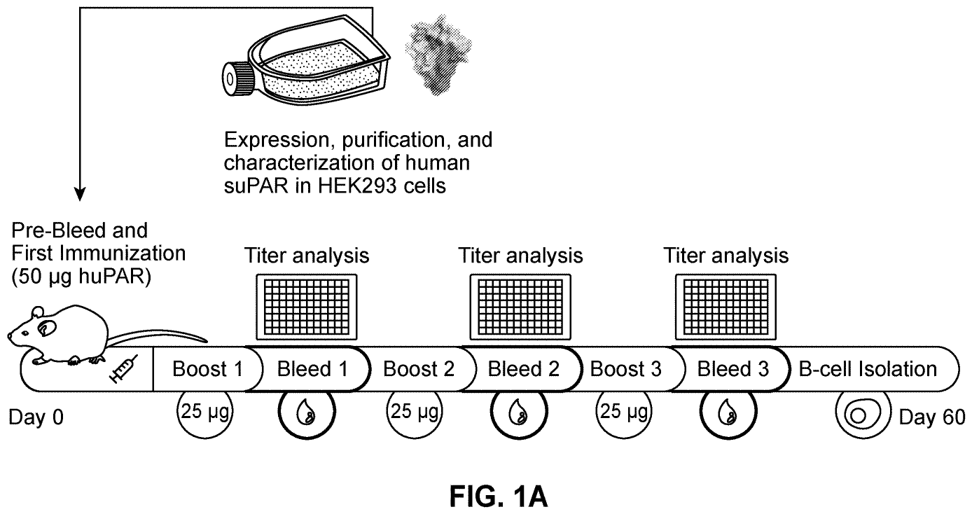

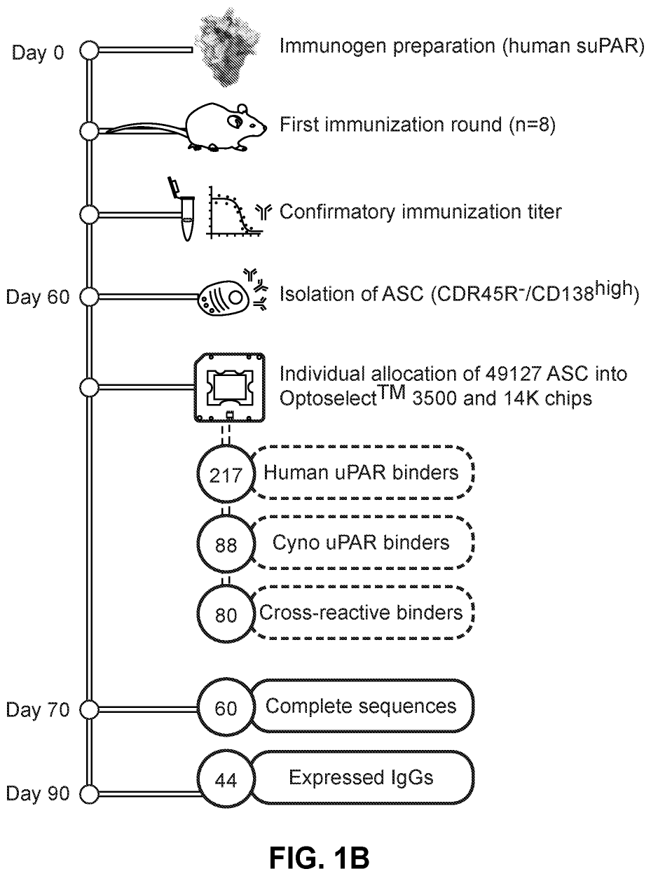

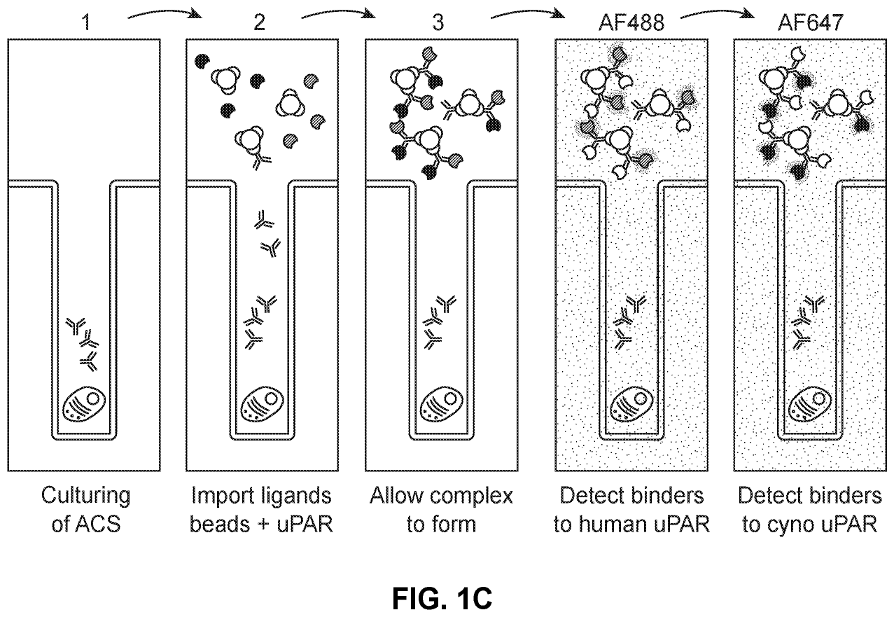

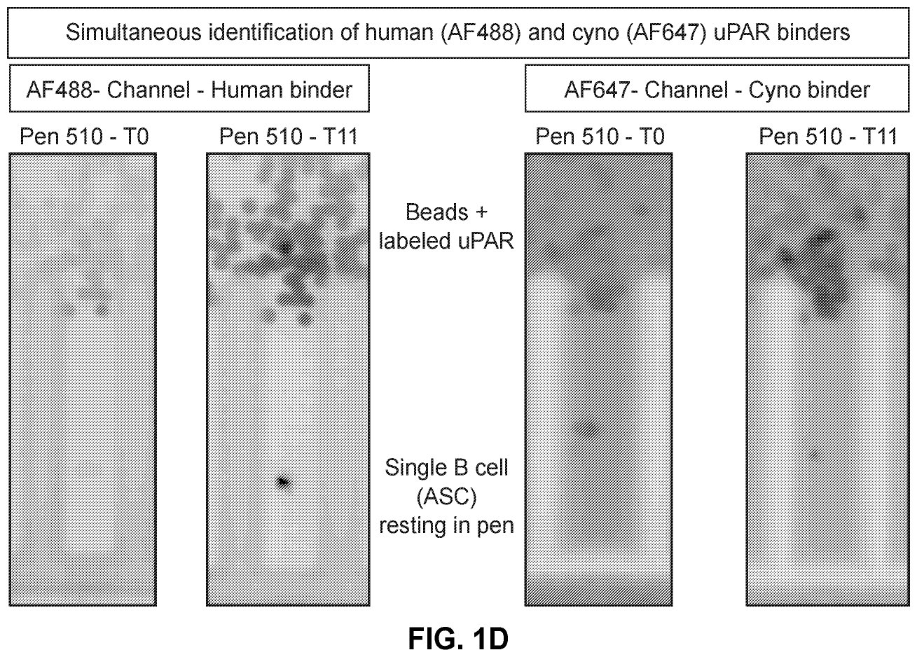

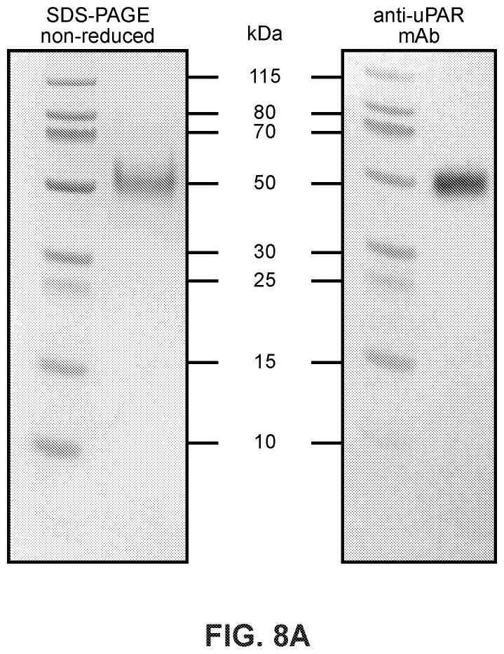

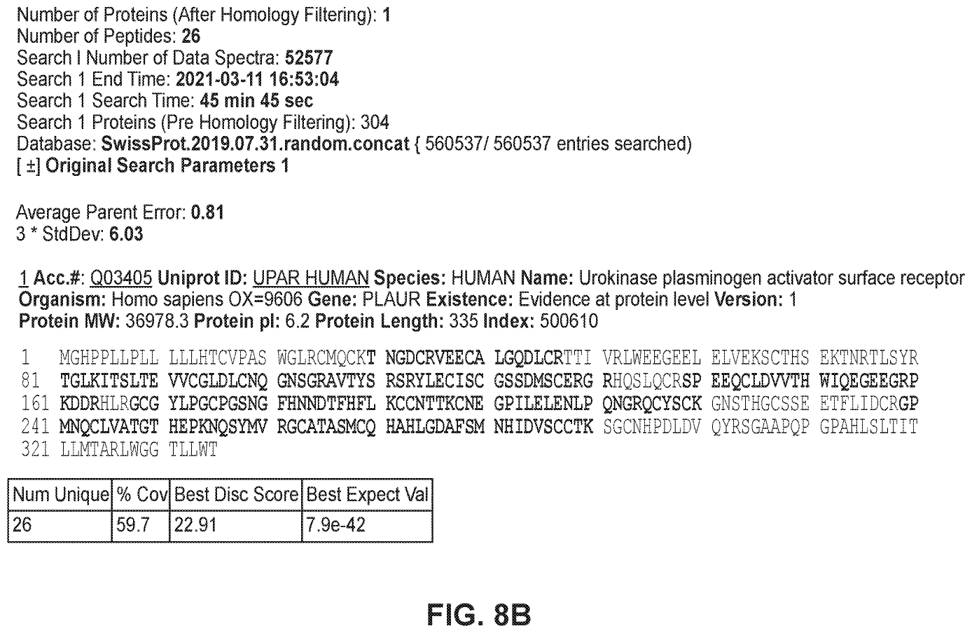

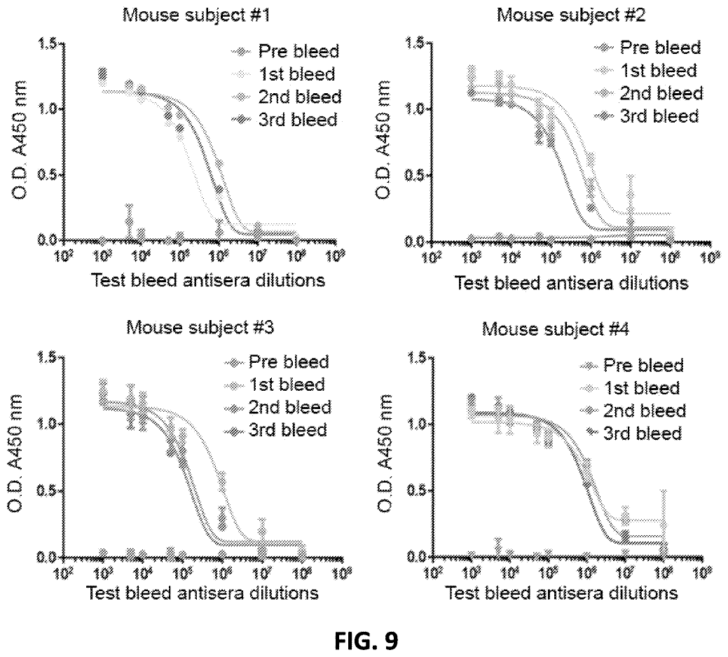

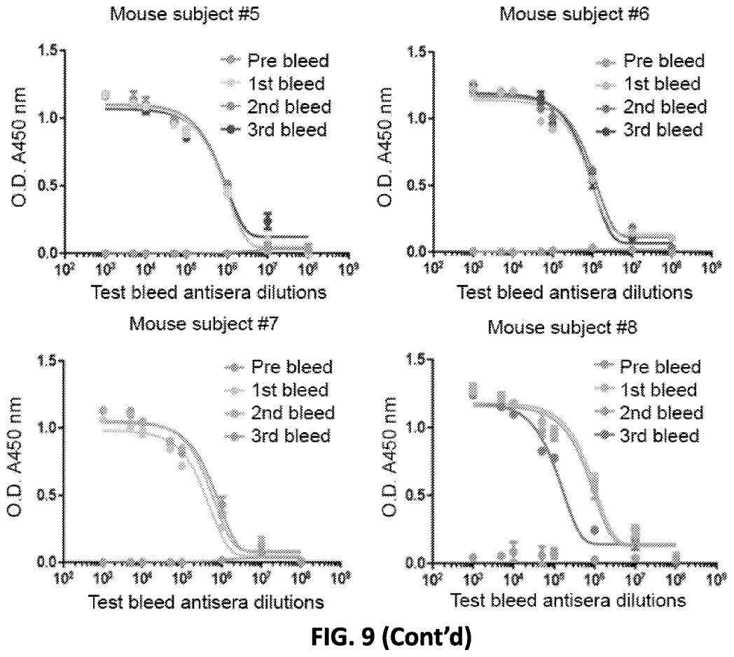

Swiss Jim Lambert (SJL/J) mice (n=8) were subjected to a 60-day immunization campaign by using a recombinant soluble form of human uPAR (suPAR), which lacks a GPI anchor, as an immunogen with seven buffering days between bleeds and boosts (FIG. 1A). The suPAR was prepared by endotoxin removal to reduce non-specific pyrogenic reactions to immunized animals, and further characterization was performed using SDS-PAGE, immunoblot, and LC-MS/MS (FIG. 8A-8B). Immunized mice were monitored by bi-weekly bleeds, followed by the determination of their antibody titers. Antisera binding curves demonstrated increased production of anti-uPAR antibodies within the first week of immunization. Sustained antibody production was maintained throughout the campaign with an antibody titer saturating at a 1×10⁷ dilution of mouse antisera (FIG. 9). With the confirmed maturation of uPAR-primed plasma B-cells, spleens and bone marrow from each animal were harvested to allow the isolation of CD45R(B220)/CD138^{high} antibody-secreting cells (ASCs) using magnetic beads and flow-assisted cell sorting.

To screen and select cross-reactive antibodies against human and cyno uPAR, high-throughput optofluidic screening of single B cells was performed using the Beacon™ platform (30). The in vivo development of antibodies relies on the maturation and selection of ASCs, offering high specificity of antibodies towards their targets with low off-target binding to other host proteins. The Beacon platform enabled the screening and selection of thousands of B cells from immunized animals, therefore, accelerating the antibody discovery process (31). A total of 49,127 mouse ASCs were imported into nanopens on OptoSelect™ 3500 chips, and screened against human, mouse, and cyno uPAR. Overall, 217 binders were identified against human uPAR, from which 80 were cross-reactive to cyno uPAR, and no cells were able to produce reactive binders to mouse uPAR (FIG. 1B-D). Interestingly, 8 ASCs produced specific binders to cyno uPAR, and 137 ASCs were specific to human uPAR.

### Example 2—VH/VL Sequencing, Cloning, and Recombinant IgG Expression

A total of 217 individual mouse B cells were exported from the Beacon, and 80 pairs of VH and VL sequences from cross-reactive binders against human and cyno uPAR were then covered using rapid amplification of cDNA ends (RACE) protocol (32). A total of 64 clones showed the amplicons within 500-700 bp with 78% recovery and these amplicons were sequenced using next-generation sequencing (NGS), resulting in 60 unique pairs of VH and VL sequences with 94% sequence recovery and 100% diversity.

Previous studies have shown that Herceptin, a humanized IgG1 monoclonal antibody that targets the HER2 protein, is able to promote tumor cell death by evoking ADCC through the interaction of its IgG1 Fc and Fcγ receptors on human immune cells (33-35). In order to convey this effector function to the anti-uPAR antibodies, 60 unique mouse VH/VL sequences were linked to the Herceptin IgG1 constant region for producing rAbs in a chimeric antibody format. From the recovery pool, 44 initial antibodies were successfully expressed recombinantly.

### Example 3—Cell-Surface uPAR Recognition and Binding Affinity of Antibody Candidates

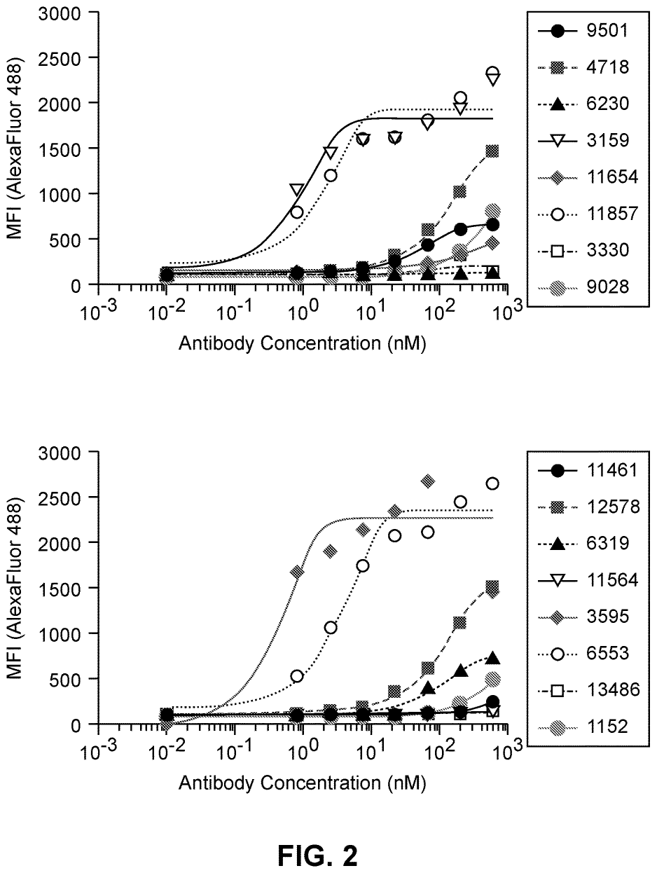

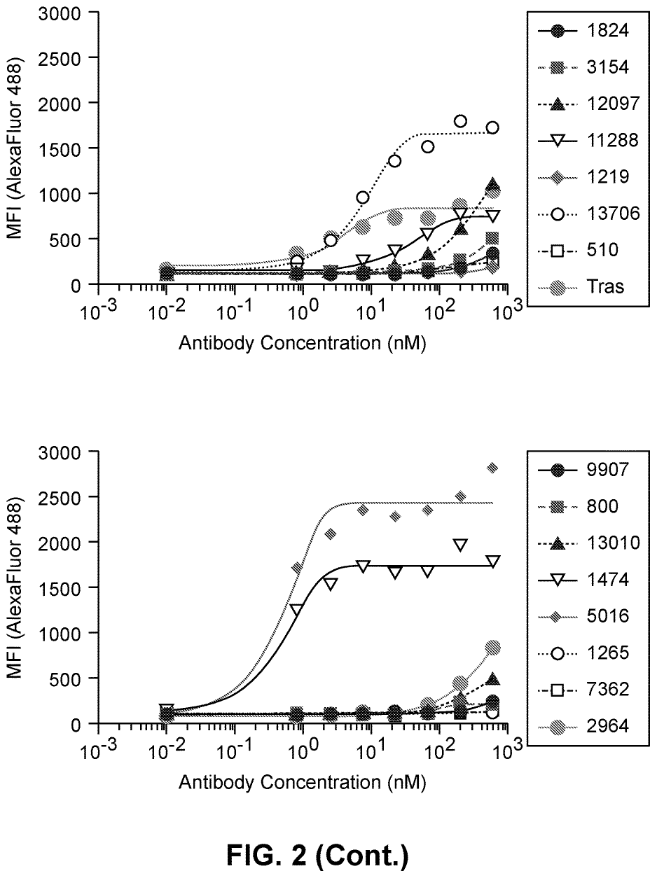

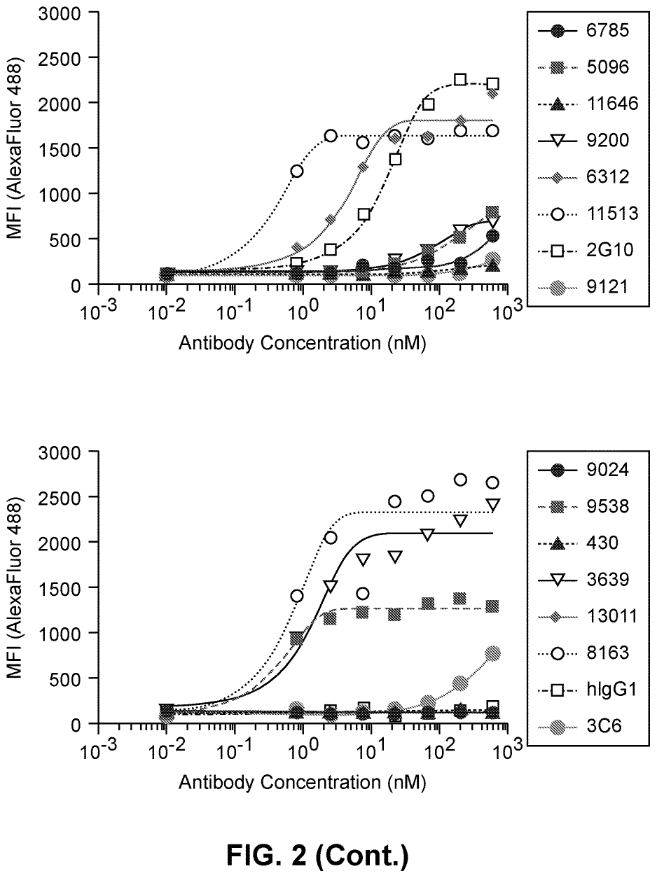

Antibodies generated by immunizing animals with suPAR, which lacks the cell surface anchoring motif, can target protein regions that are not accessible for membrane-bound uPAR. Nonetheless, effective targeting of cell surface receptors benefits from recognizing both solvent-exposed epitopes and also native conformational states displayed on the cell surface (36). Thus, FACS was applied to evaluate the cell-surface uPAR recognition by each antibody candidate under different concentrations of MDA-MB-231 cells, a triple-negative breast cancer cell line with high uPAR expression (FIG. 2). From the 44 initial antibodies, 12 lead candidates recognized uPAR displayed on breast cancer cells in a dose-dependent manner, with half-maximal effective binding concentration (EC₅₀) values ranging from 0.39 to 7.6 nM (FIG. 2). All candidates were benchmarked against 2G10 and 3C6, and were shown to be more potent at recognizing cell-surface uPAR as demonstrated by their lower EC₅₀ values, and these values are comparable to Herceptin binding to HER2 (EC₅₀=3.6 nM) (Table 2) (37,38).

TABLE 2
In vitro characterization of novel anti-uPAR antibody candidates.

| | Cellular | | Human uPAR | Cyno uPAR | ADCC | | ADCC | | ADC | | Adhesion |
|---|---|---|---|---|---|---|---|---|---|---|---|
| Antibody | recognition | | binding | binding | NK-92 | | PBMCs | | Cytotoxicity | | blocking |
| Candidates | | EC₅₀ | EC₅₀ | EC₅₀ | | EC₅₀ | | EC₅₀ | | EC₅₀ | EC₅₀ |
| ID number | Max % | (nM) | (nM) | (nM) | Max % | (nM) | Max % | (nM) | Max % | (nM) | (μM) |
| 1474 | 1740 | 0.506 | 0.14 | 0.20 | 15.56 | 0.73 | — | — | — | — | — |
| 3159 | 1830 | 0.9158 | 0.53 | 0.27 | 28.18 | 0.887 | 47.41 | 0.11 | 32.02 | 0.75 | 1.61 |
| 3595 | 2281 | 0.453 | 0.05 | 0.10 | 21.85 | 0.2 | 55.59 | 0.10 | 34.76 | 0.66 | >10 |
| 3639 | 2086 | 1.415 | 0.52 | 0.19 | 34.09 | 5.948 | 57.06 | 0.53 | 2.32 | NA | >10 |
| 4718 | 1500 | 120.9 | — | — | 4.273 | 0.55 | — | — | — | — | — |
| 5016 | 2429 | 0.578 | 0.75 | 0.11 | 23.73 | 3.119 | 49.56 | 0.12 | 0 | NA | >10 |
| 6312 | 1809 | 4.53 | 0.28 | 0.21 | 16.44 | 4.03 | — | — | — | — | 3.38 |
| 6553 | 2353 | 3.76 | 0.33 | 0.21 | 17.44 | 5.6 | — | — | — | — | >10 |
| 8163 | 2330 | 0.6902 | 0.41 | 0.15 | 18.81 | 1.135 | 57.55 | 0.09 | 28.7 | 0.57 | 5.40 |
| 9538 | 1270 | 0.4602 | 0.67 | 1.12 | 29.71 | 50.2 | 45.64 | 13.01 | 4.26 | NA | 3.51 |
| 11513 | 1645 | 0.388 | 0.75 | 0.28 | 21.6 | >200 | — | — | — | — | — |
| 11857 | 1936 | 2.07 | 0.52 | 0.18 | 27.16 | 5.59 | 52.04 | 0.35 | 35.16 | 0.76 | 0.94 |
| 12578 | 1556 | 109.5 | — | — | 16.04 | 49.48 | — | — | — | — | — |
| 13706 | 1669 | 7.606 | 0.53 | 0.30 | 54.56 | 4.851 | 65.66 | 0.79 | 3.24 | NA | >10 |
| 2G10 | 2204 | 16.65 | 0.26 | NA | 33.92 | 22.53 | 65.45 | 8.65 | 4.9 | NA | 3.5 |
| 3C6 | 971 | 291.9 | 0.28 | NA | — | — | — | — | 1.42 | NA | 0.81 |
| hIgG1 | 155.3 | — | — | — | 0.468 | NA | 4.8 | NA | | | — |
| Trastuzumab | 841.8 | 3.647 | — | — | — | — | — | — | | | — |

NA = No Applicable Activity,
Dash (—) = not determined

The binding affinity of lead candidates to human uPAR was further characterized using biolayer interferometry (BLI). All lead candidates bound to human uPAR with equilibrium dissociation constant (K_{D}) values in the pM range, exhibiting stronger binding affinity than 2G10 and 3C6 in the double-digit nanomolar range (17). These results demonstrated the power of selecting in vivo, affinity matured antibodies to develop tight binders with slow off rates against uPAR.

### Example 4—Cross-Reactivity Profiles Confirmed by ELISA

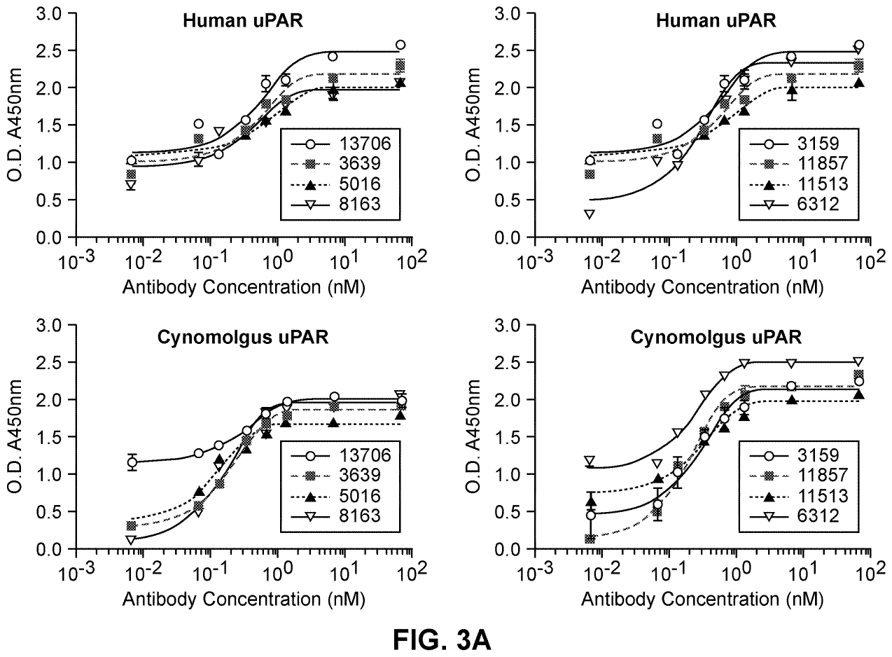

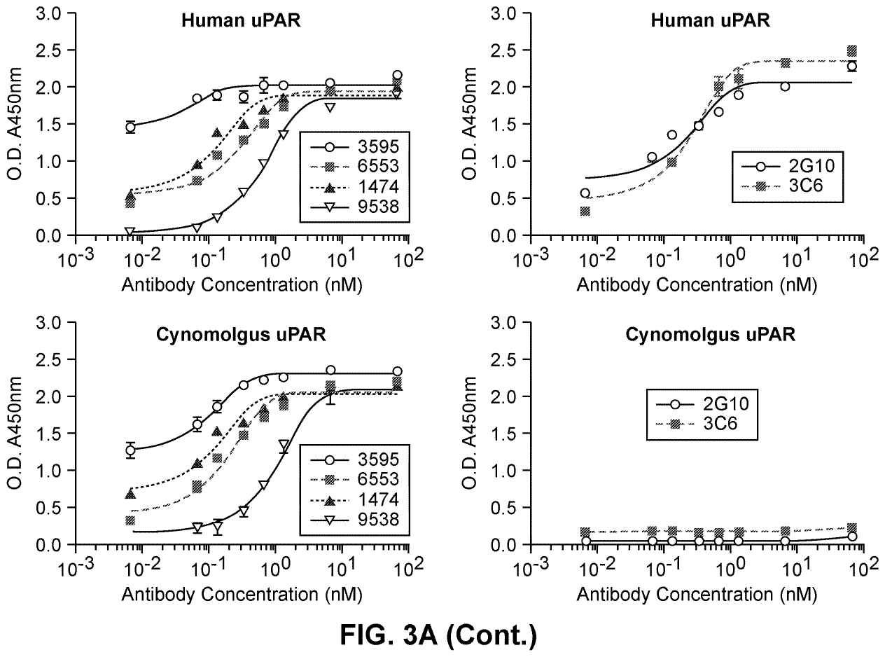

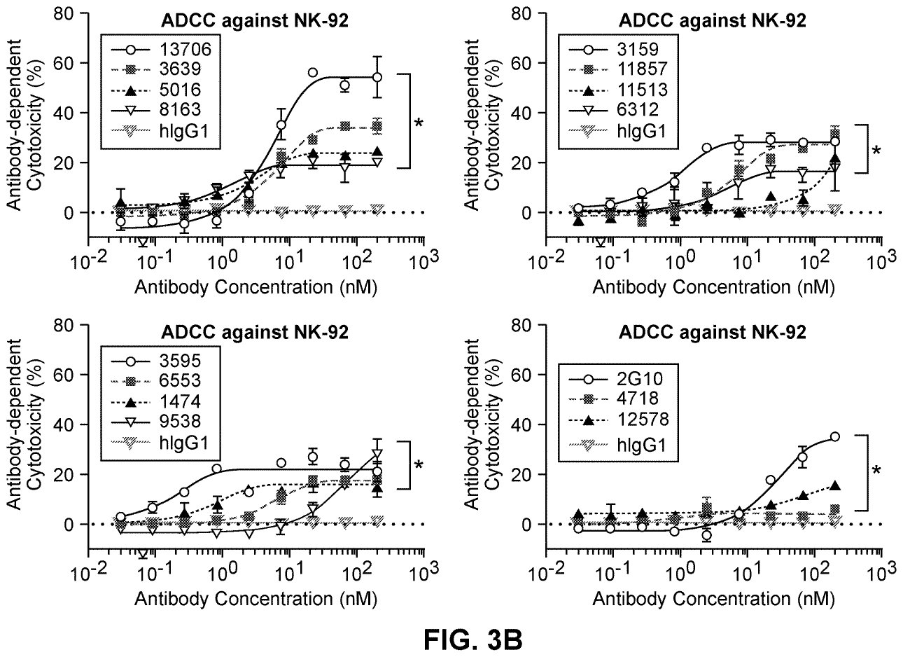

To ensure the cross-reactivity of the 12 lead candidates was maintained with the Herceptin constant region, their binding to human and cyno uPAR was evaluated by ELISA (FIG. 3A). The results demonstrated that all lead candidates displayed cross-reactivity and showed strong binding to both human and cyno uPAR with EC₅₀ values ranging from 0.05-0.8 nM and 0.1-1.1 nM, respectively. Two of them exhibited approximately 2-fold higher binding affinity to human uPAR, seven antibodies showed 2-7 fold stronger binding to cyno uPAR, and three antibodies bound to human and cyno uPAR with comparable binding affinity. On the other hand, 2G10 and 3C6 were able to bind to human uPAR, but no reactivity was observed with cyno uPAR (FIG. 3A and Table 2).

### Example 5—Antibody-Dependent Cellular Cytotoxicity (ADCC)

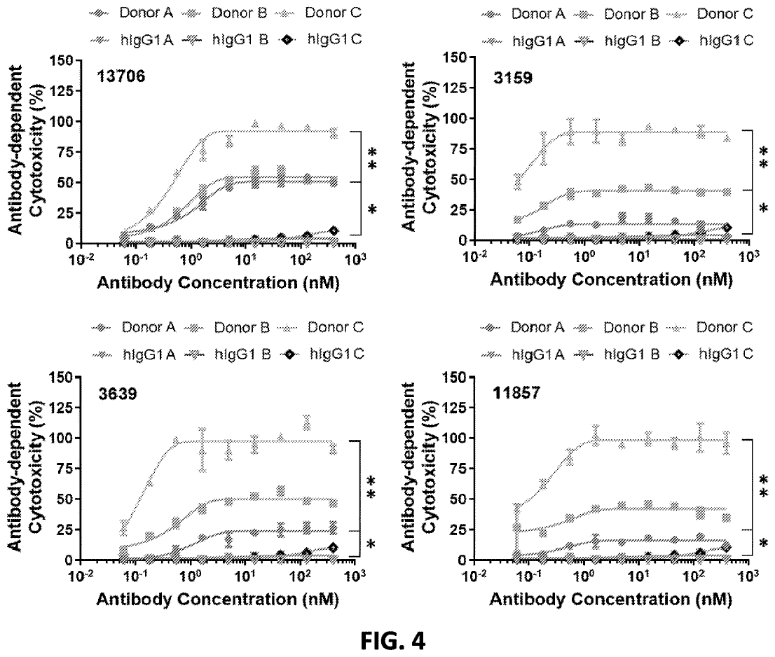

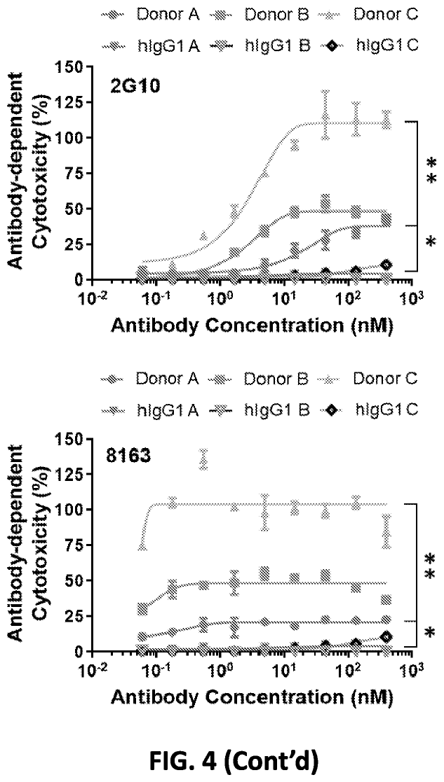

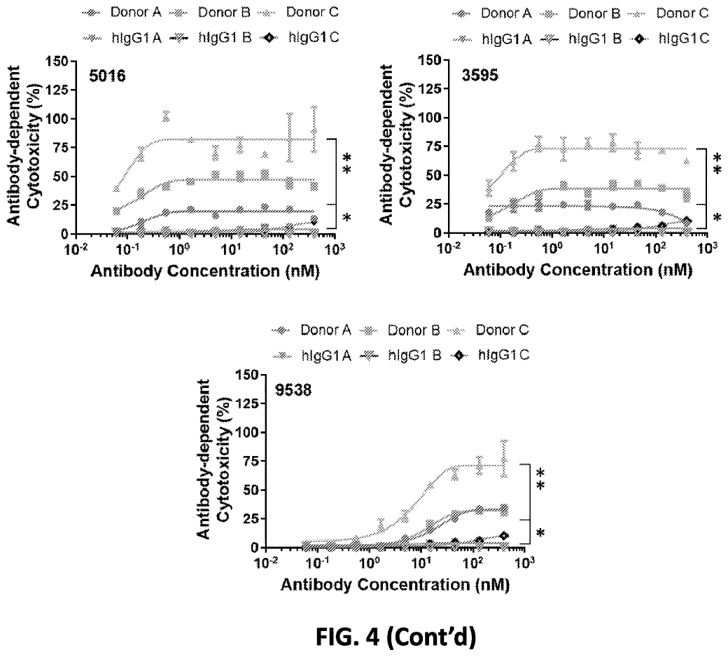

With the confirmation of cell-surface uPAR recognition and cross-reactivity of the 12 lead antibody candidates, evaluated next was whether they mediate ADCC to promote tumor cell death as a uPAR-targeted immunotherapy approach. The ADCC assay for all lead antibody candidates was first performed using MDA-MB-231 cells as target cells in the presence of NK-92 MI CD16a effector cells. The resulting dose-response curves demonstrated that all lead candidates, except 4718, were able to induce ADCC, and no cytotoxicity was observed for the isogenic huIgG1 control (FIG. 3B). Selected eight antibody candidates (3159, 3595, 3639, 5016, 8163, 9538, 11857, 13706), which exerted effective ADCC response in NK-92 cells, were further tested for ADCC activity in the presence of human PBMCs from three different healthy donors. Due to the intrinsic characteristics of effector cells from each PBMC donor, this often results in considerable donor-to-donor variability in their capacity to induce ADCC (39). The inherent donor variability was observed as expected between eight lead candidates, and the results showed that they all facilitated effector cell function against MDA-MB-231 cells in a dose-dependent manner in the presence of healthy PBMCs (FIG. 4). The mean maximum percentage of ADCC response for eight antibodies ranges from 46 to 66%, and seven of them are more potent than 2G10 with 10 to 96-fold lower EC₅₀ values from 0.1 to 13 nM (Table 2).

### Example 6—Cytotoxicity as Antibody Drug Conjugates (ADCs)

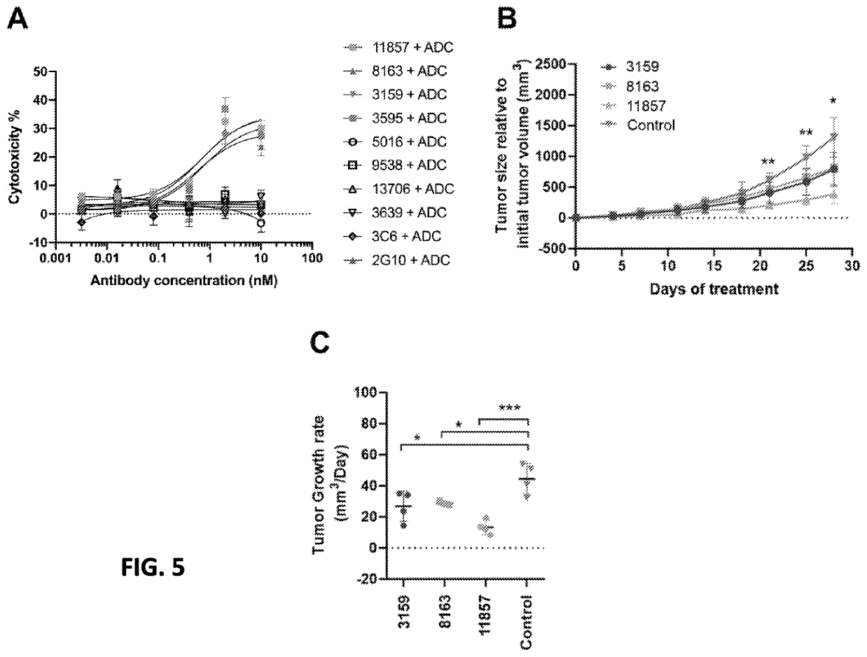

In addition to Fc-mediated ADCC as a strategy for providing antitumor cytotoxicity, antibody-drug conjugates (ADCs) have been developed rapidly in recent decades to selectively deliver cytotoxic payloads directly to the target cancer cells (40). To determine if the selected eight antibody candidates can be internalized by targeting uPAR for the ADC approach, ADC efficacy was assessed in vitro against the MDA-MB-231 cells with Fab-αHFc-CL-MMAE, which recognizes the Herceptin Fc moiety and has a cathepsin-cleavable linker connecting to monomethyl auristatin E (MMAE). The control was performed without the treatment of @HFc-CL-MMAE, and no cytotoxicity was observed. Although all antibodies recognized cell-surface uPAR, only four of them (3159, 8163, 11857, and 3595) exhibited a concentration-dependent increase in ADC cytotoxicity in the presence of αHFc-CL-MMAE. The low EC₅₀ values of the four antibodies range from 0.57 to 0.76 nM indicating they target uPAR in a distinct complex and induce efficient internalization (FIG. 5A).

### Example 7—Inhibition of Cell Adhesion to Vitronectin

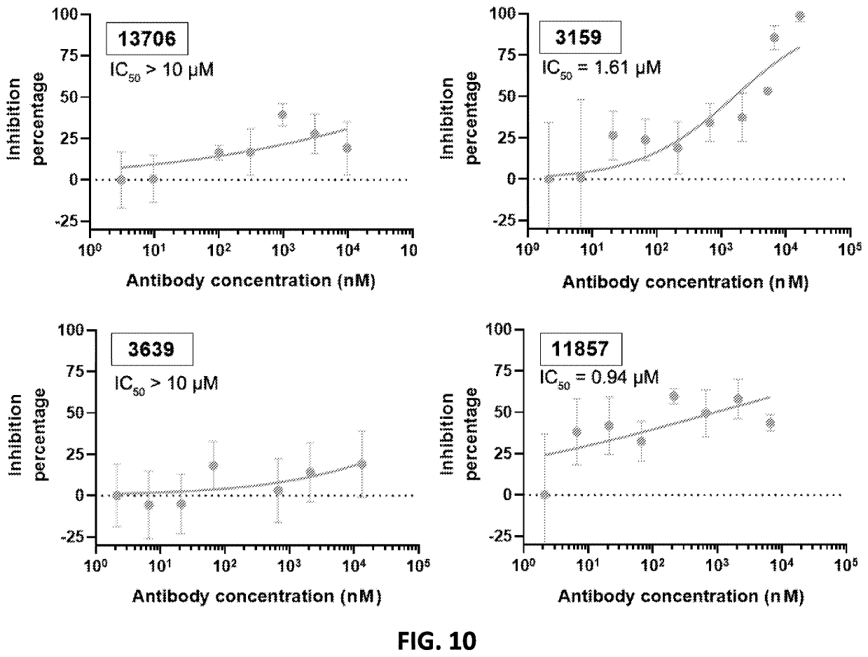

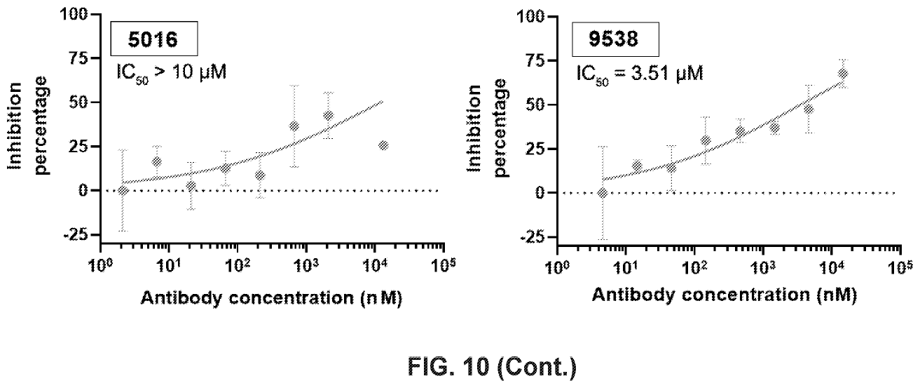

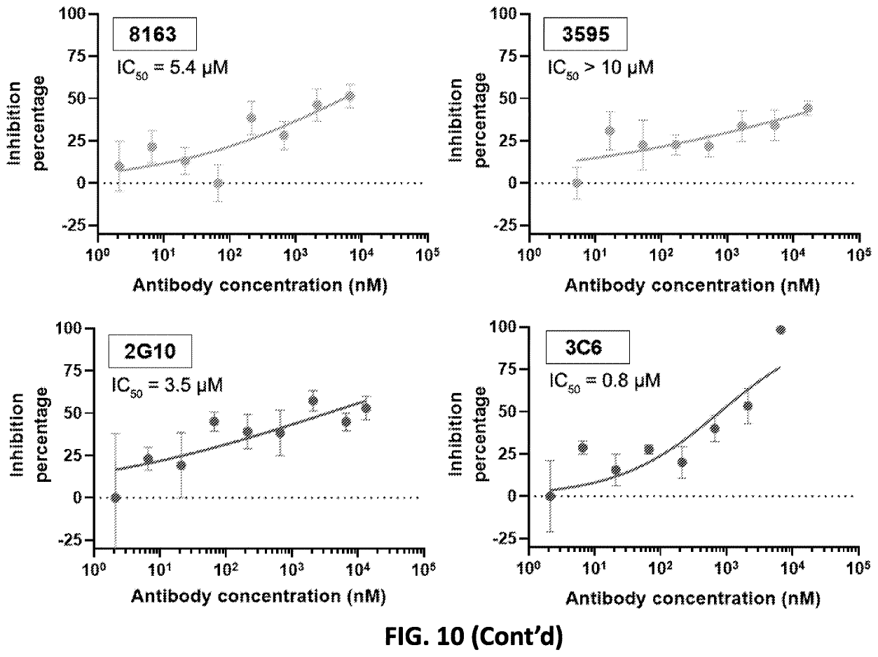

The binding of vitronectin (VN) to uPAR is known to induce intracellular signaling events that activate integrins to promote cancer cell adhesion and communication to the extracellular matrix (41). To investigate whether the eight lead antibody candidates have any functional inhibition on the tumor cell by targeting cell-surface uPAR, their ability to block uPAR-mediated cell adhesion to VN was evaluated. The results demonstrated that the eight lead candidates were able to inhibit the adhesion of MDA-MB-231 cells to VN-coated wells in a dose-dependent manner, and five of them (3159, 6312, 8163, 9538, and 11857) had EC₅₀ values from 0.9 to 5.4 μM (Table 2 and FIG. 10). Candidate 3159 showed the strongest inhibitory effect and is comparable to 3C6, which was identified as an inhibitor to abrogate uPAR-mediated cell adhesion in a previous study (42). Overall, the in vitro characterization of the lead antibodies highlighted candidates 3159, 8163, and 11857 as the most promising rAbs with ADCC activity, ADC cytotoxicity by inducing efficient uPAR-rAb internalization, and the functional inhibition on cell adhesion (Table 2), prompting an investigation into their therapeutic efficacy in an orthotopic animal model of breast cancer.

### Example 8—Therapeutic Efficacy in an Orthotopic Animal Model of Human Breast Cancer

To determine the in vivo therapeutic efficacy of the 3 lead antibodies, MDA-MB-231 cells were orthotopically implanted in the mammary fat pads of Foxn1^{nu} nude mice, and animals with tumors (75-100 mm³ in volume) were treated weekly via intravenous injection with each antibody (30 mg/kg). Close monitoring of tumor growth across treatment groups revealed that all antibodies were able to reduce tumor burden relative to the untreated control (FIG. 5B). The data demonstrated candidate 11857 exhibited the most potent efficacy with a 3.1±0.4 fold smaller tumor burden on day 21 (p=0.0039) compared to the untreated control. Such activity was maintained on Days 25 and 28 with a 3.3±0.3 (p=0.0058) and a 3.6±0.6 (p=0.0125) fold smaller tumor burden than that of the untreated control group, respectively (FIG. 5B). Although candidates 3159 and 8163 had little effect in reducing tumor volume in comparison to candidate 11857, they were nonetheless effective in reducing tumor growth rates relative to the untreated control (FIG. 5C). The superior antitumor activity of candidate 11857 was also reflected on its ability to impair tumor growth rates in comparison to the untreated control (p=0.0002), which was far superior to that of candidates 3159 (p=0.0141) and 8163 (p=0.0267) (FIG. 5C).

### Example 9—Epitope Binning Using Biolayer Interferometry

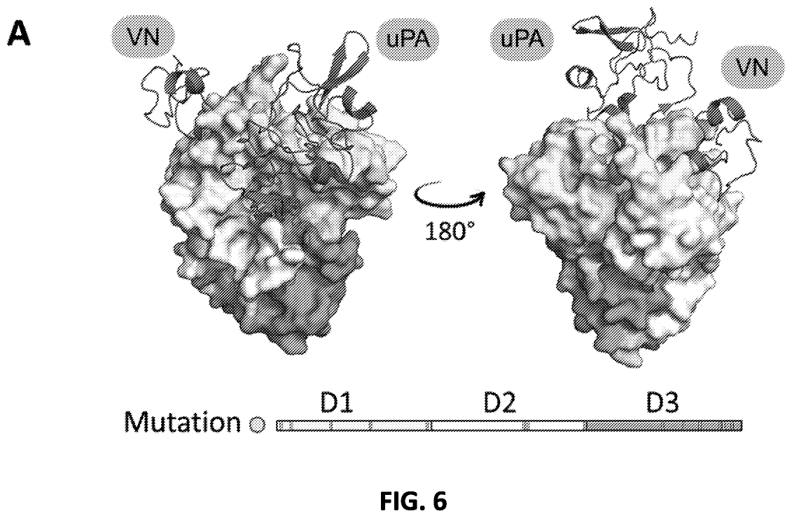

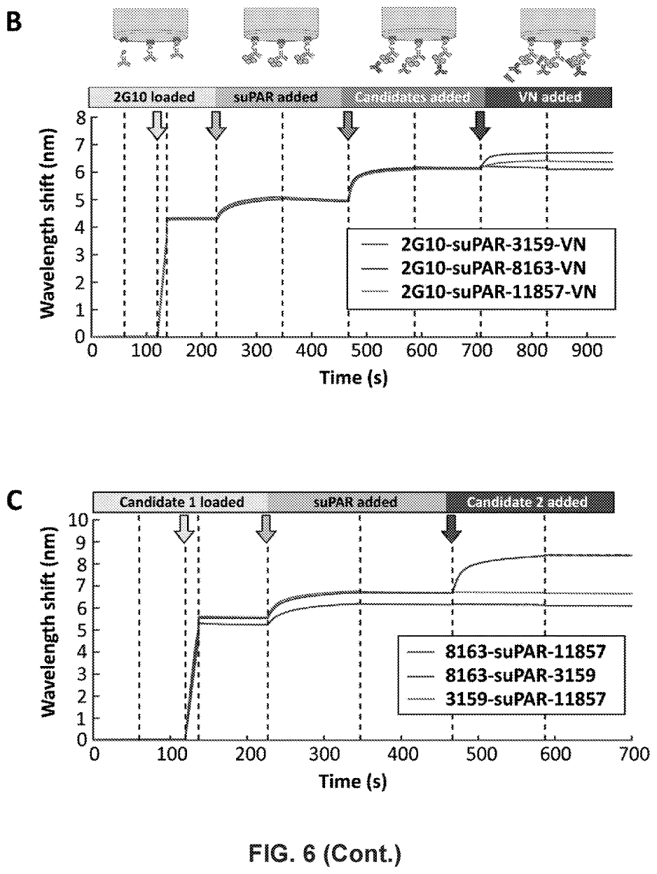

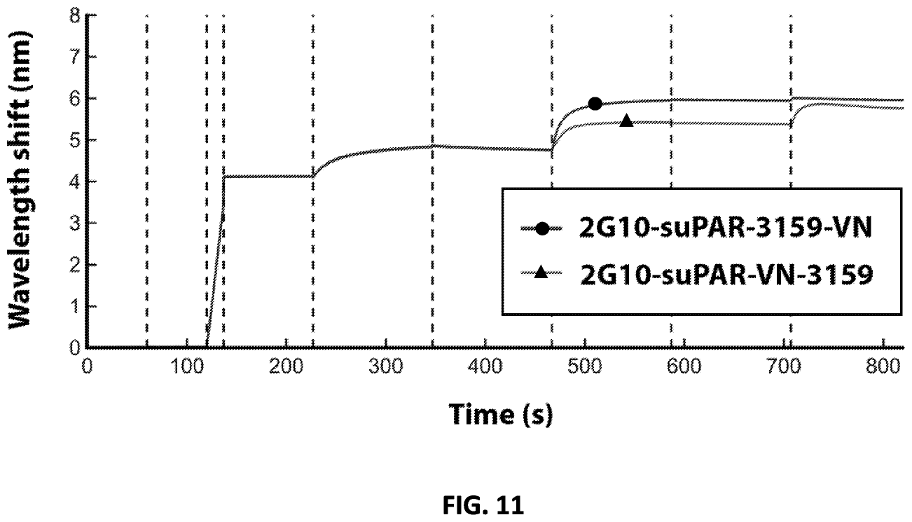

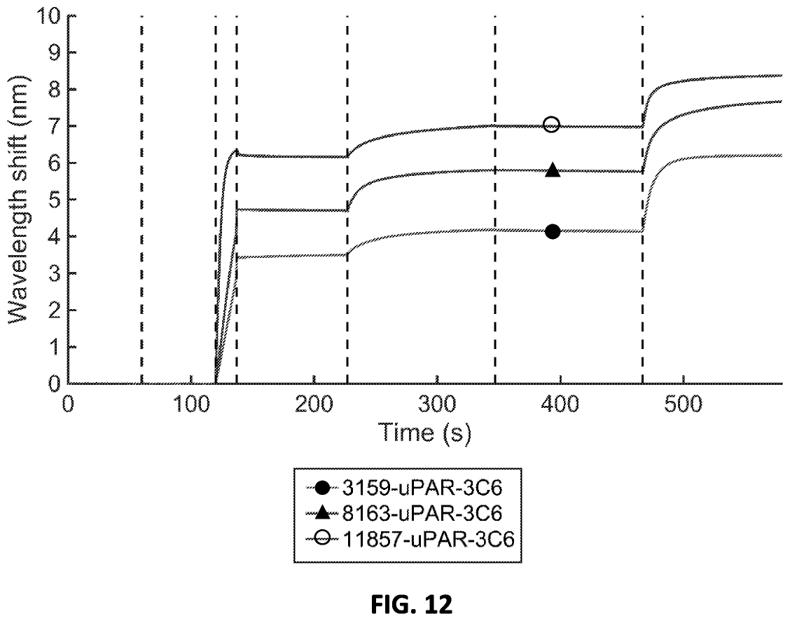

The decrease in tumor growth rate along with the ability of the three lead antibodies (3159, 8163, and 11857) to impair cell adhesion led us to investigate their binding epitopes on uPAR. The epitope binning was performed by BLI, and 2G10 was included as a control. Human and cyno uPAR share 96% sequence identity (FIG. 6A), where most of the sequence variation between the homologs lies at the uPA-binding site resulting in the species-specific interaction between uPA and uPAR (43,44). On the other hand, the VN-binding site is located on the opposite side and is more conserved between uPAR homologs (FIG. 6A). A previous study has shown that 2G10 was identified as a competitor to disrupt the uPA/uPAR interaction, suggesting it binds to a region that prevents uPA binding (45). The BLI curves indicated the three lead antibodies can bind to uPAR after the formation of the 2G10-uPAR complex. This result demonstrated they target uPAR at the regions that are distinct from the uPA-binding domain where 2G10 is recognized (FIG. 6B). To further test whether the three lead antibodies could inhibit the binding of VN to uPAR, the binding response of VN after each of them was bound to uPAR was measured (FIG. 6B). BLI measurements showed the binding of 3159 to uPAR completely abolished subsequent VN binding, 11857 displayed a partial effect on blocking the binding of VN, and 8163 did not affect VN binding to uPAR (FIG. 6B). This is consistent with the result from the adhesion assay showing 3159 exhibited the strongest inhibition on VN-mediated cell adhesion, suggesting 3159 recognized the VN-binding site and blocked the interaction between VN and uPAR. The result was also confirmed by changing the order of adding 3159 and VN to show that 3159 was able to compete with VN for binding to uPAR (FIG. 11). Furthermore, an epitope competition assay was performed in different pairs of antibodies to identify if they have distinct binding epitopes (FIG. 6C). Interestingly, both 3159 and 8163 could bind to uPAR at the same time, but neither one of them was able to interact with uPAR once a rAb-uPAR complex was formed with 11857, suggesting 11857 has a partially overlapping epitope with 3159 and 8163 (FIG. 6C). In addition, it was also found the binding epitopes of these three antibodies are distinct from 3C6 that is known to block the integrin aVB1 (FIG. 12).

### Discussion

The growing understanding of uPAR and its molecular partners in tumorigenesis, cancer progression, and metastasis has provided the basis for developing novel diagnostic, prognostic, and therapeutic approaches to treat a wide variety of tumors (46,47). Cynomolgus monkeys have served as a valuable model to provide the most relevant information on the safety, efficacy, and pharmacokinetic profiles of translational therapeutics for human use (48,49). Thus, this study presented a rapid antibody discovery pipeline that would allow the identification of cross-reactive antibodies against human and cyno uPAR. The 60-day immunization campaign using recombinant human suPAR enabled a rapid generation of uPAR-primed B cells in SJL mice, and the Beacon platform allowed the culture, manipulation, and screening of single B cells in one day, with 99% assurance of clonal origin. A similar approach has recently been used for the successful development of neutralizing antibodies against SARS-COV and SARS-COV-2 (50). The present approach provides an example of using immunization to bias the immune response coupled to the screening of antigen-primed B-cells for identifying human/cyno cross-reactive antibodies with strong binding affinity and antitumor activity against human breast cancer, showing the power in vivo development and affinity maturation in B cells for antibody selection.

Effective tumor-targeting antibodies induce direct and indirect effects on tumor cells, mediated by their Fab variable regions and Fc constant regions, respectively (51). The targeted therapies for HER2-positive breast cancer in clinical use (i.e. Herceptin and Perjeta) target the HER2 protein and induce ADCC by recruiting immune effector cells through the Fc domain as part of their mechanism for tumor-killing (52). To confer the mouse antibodies with ADCC activity exerted by human immune cells, their VH/VL domains were engineered to include the Herceptin constant regions. Interestingly, different amplitudes of ADCC were observed in the presence of NK-92 cells, suggesting the epitope recognition of antibodies is critical to modulate ADCC activity. These findings corroborate previous studies which demonstrated how antigen binding can alter an IgG conformation and affect the recognition of the Fc region by the FcγRIIIa and FcγRIIIb receptors on the surface of NK cells and PBMCs (53-55). In addition, the binding epitope of an antibody affects the angle of its Fc domain relative to the target cell surface and could govern the accessibility of the Fc region for the interaction with effector cells to induce ADCC (56,57).

Although ADCC is one of the primary mechanisms for most antitumor mAbs currently in the clinic, recent findings suggest that antibodies, that functionally inhibit their targets while inducing ADCC, offer further benefit to achieve an effective anti-tumor response (58,59). Previous studies have shown how a vitronectin deficiency strongly impairs tumor growth in an orthotopic xenograft model of breast cancer (60). In addition, the binding of uPAR to vitronectin has been shown to regulate cell adhesion and further trigger changes in cell morphology, migration, and signaling (61-63). A reported mAb 8B12 was found to inhibit the vitronectin binding to uPAR and effectively reduce uPAR-mediated cell migration on vitronectin-coated surfaces (18). These studies revealed how inhibiting the interaction of cancer cells with the ECM can affect their pro-proliferative communication in the tumor microenvironment and overall tumorigenesis. This supports the finding that the inhibition of cell adhesion found in the lead candidates provides an advantage to impair tumor growth along with their ability to induce ADCC (41,64).

In addition to ADCC, several therapeutic antibodies have been redeveloped as ADCs to deliver cytotoxic drugs to antigen-positive tumor cells (65). Here, the lead antibodies were evaluated in ADC cytotoxicity and showed the potential of achieving cytotoxicity in MDA-MB-231 cells. According to previous studies, uPAR can be internalized by tumor cells via clathrin-mediated endocytosis or via a clathrin-independent mechanism mediated by LRP-1, both of which are responsible for trafficking uPAR to the lysosome for degradation and recycling (66,67). This could provide an additional advantage because all known internalization mechanisms of uPAR cause it to dissociate from its co-receptors, which include matrix-engaged integrins and other bonafide ligands, and therefore abrogate the downstream signaling (68).

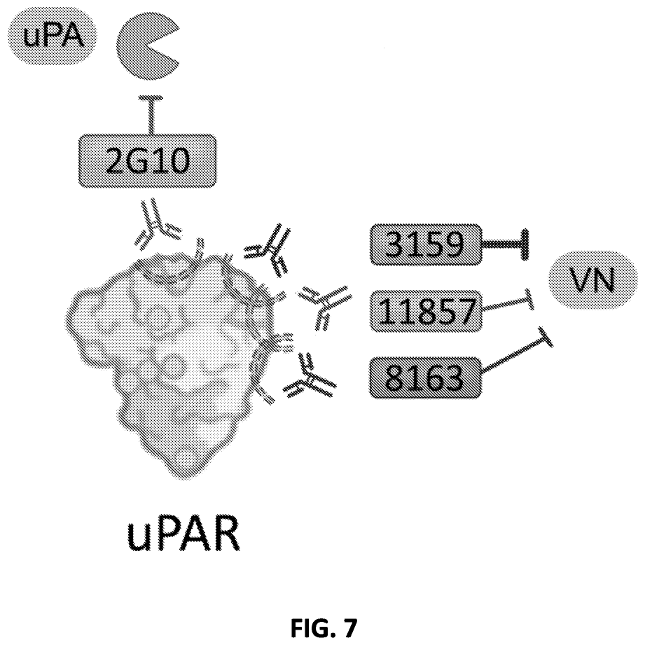

Finally, based on the biolayer interferometry data and inhibition assays, a binding model (FIG. 7) is proposed for the three lead antibodies to uPAR. Candidates 3159, 8163, and 11857 bind to uPAR with distinct epitopes from the previously reported binders, 2G10 and 3C6, and their binding epitopes are located on the opposite side of the central uPA-binding cavity. The binding sites of 3159 and 8163 on uPAR are independent, and the 11857 binding epitope overlaps considerably with their binding sites but is not identical. All of them exhibited an inhibitory impact on cell adhesion, and 3159 binds to an epitope on uPAR for vitronectin binding and therefore showed the strongest inhibitory effect. 11857 binds to a spot resulting in a synergistic effect on ADCC, uPAR internalization, and blocking cell adhesion, showing the advantage of having antibodies with ADCC and additional functional effects for impairing tumor growth.

### Materials and Methods

### Antigen Production

A HEK293 cell line, stably expressed suPAR, was generously provided by the Chapman Lab at UCSF. Cells were grown in DMEM complete medium supplemented with 10% fetal bovine serum (FBS), 100 U/mL penicillin, and 100 μg/L streptomycin (Gibco) at 5% CO₂ and 37° C. For suPAR production, 3.7×10⁷ cells were seeded in a 5-stack Corning® CellSTACK® Culture Chamber and maintained in the complete medium. The proteins were harvested at day 3 and purified using a Ni-NTA column followed by gel-filtration using a Hiload 16/600 Superdex200 prep-grade column. The production of suPAR was confirmed by immunoblot analysis using a mouse anti-human uPAR monoclonal antibody clone R-3 (Invitrogen) and an HRP-conjugated goat anti-mouse IgG (H+L) antibody (Biorad). Purified suPAR samples were also characterized by LC-MS/MS and further subject to endotoxin removal using Pierce™ High-Capacity Endotoxin Removal Spin Columns. After endotoxin removal, samples were characterized for their total endotoxin level and only those not exceeding 0.5 U/mL were approved for the preparation of immunogen injections.

### Animal Immunization Strategy

The immunization strategy consisted of a primary intraperitoneal injection with 50 μg of antigen prepared as an emulsion in Freund's Complete Adjuvant (FCA). Nine Swiss Jim Lambert (SJL) mice (6-8 weeks old) received a boost injection containing 25 μg of antigen every other week for three boosts, with bleeds being intercalated to each boost injection for 60 days. A pre-bleed was performed for each mouse prior to the start of the immunization campaign in order to control for non-anticipated exposure to the antigen. All blood samples were allowed to clot and 100-200 μL of serum was stored at −80° C. for antibody titer determination.

### Harvest and Enrichment of Mouse Antibody-Secreting Cells (ASCs)

Animals were euthanized in accordance with approved IACUC protocols. Spleens and bone marrows were harvested and processed into single-cell suspension in RPMI. Total B-cells were isolated by magnetic negative selection using EasySep Mouse Pan-B Cell Kit (StemCell) to deplete non-B cells from the single-cell suspension. Antibody-secreting cells (ASCs) were enriched from single B cell suspensions by magnetic positive selection using EasySep Mouse CD138+Kit (StemCell) and a FACS-based positive selection through the gating of CD45R (B220)/CD138^{high} cells, which are traditionally used to broadly define the population of plasma cells (69).

### Nanofluidic Optoelectro Screening of Single B Cells

Direct screening of secreted antibodies from ASCs was achieved with the Beacon platform. Enriched ASCs were injected into a 0.75 nL OptoSelect™ 3500 and OptoSelect™ 14K chips. The platform enables the usage of tunable optoelectro positioning parameters to effectively isolate single ASCs into nanopens. ASCs were individually cultured for 1 hr within the chip and screened for both IgG secretion and antigen specificity using an in-channel multiplex bead-based fluorescent assay. Briefly, beads coated with rabbit anti-mouse IgG (H+L) were imported to the chip where active accumulation of secreted antibodies was identified by the binding of a FITC-labeled goat anti-mouse secondary antibody to the beads. Antigen specificity was evaluated by importing fluorescently labeled uPAR from either human (conjugated to Alexa Fluor 488), cynomolgus monkey (R&D Systems) or mouse (Sino Biologicals) (conjugated to Alexa Flour 647) into the chip. The binding of ASC-derived IgGs to the antigen was monitored by the time-dependent increase of uPAR-derived fluorescence on the beads found at the mouth of the nanopen. The selected ASC were exported into a 96-well plate containing lysis buffer. HEK293T/17 cells expressing 2G10 were used as a positive control for both IgG secretion and the production of anti-uPAR antibodies.

### Sequencing, Recombinant Cloning, Expression, and Purification of Candidate Antibodies

Single B cells were exported from Beacon to 96-well plates containing lysis buffer and mineral oil. The cDNA generation process was performed using ChemPartner & BLI's proprietary protocol using RNA capture beads. Selected human and cyno uPAR cross-reactive binders were then amplified with RACE PCR protocol using proprietary heavy and light chain constant reverse primers. Clones showed amplicons within 500-700 bp and were sequenced by next generation sequencing (NGS). The NGS library preparation was done by indexing universal forward and reverse constant primers. Then, the samples were run on MiSeq (Illumina). The raw data was analyzed using NGS-related software.

VH and VL sequences were linked to Trastuzumab constant regions and cloned into a pcDNA3.4-hCg1 or pcDNA3.4-hCk mammalian expression vectors. The transfection and expression of recombinant IgG were performed based on the manufacturer's protocol. Briefly, HEK293F cells were seeded in FreeStyle™ media and incubated at 130 rpm and 37° C. with 8% CO₂. Transfection was carried out using polyethylenimine (PEI) keeping a 1:2 ratio of DNA/PEI. A 5% solution of peptone was added at 0.1 equivalent volumes of the original cell suspension to improve recombinant protein synthesis. On day 6-7 post-transfection, the IgG-enriched media were collected, and IgGs were purified using protein A column (GE MabSelect™ SuRe™) and dialyzed overnight against PBS (pH 7.4) at 4° C.

### Biolayer Interferometry (BLI) Analysis

The binding affinity of anti-uPAR antibodies was measured using an Octet RED384 System at 25° C. Octet SA (Streptavidin) biosensors were immobilized with biotinylated human or cyno uPAR at 2 μg/mL (Protein Sciences) in assay buffer (PBS with 1% BSA). After equilibrium to baseline in assay buffer, the biosensor was put into each well containing anti-uPAR antibodies and allowed for dissociation in assay buffer. The association and dissociation curves were analyzed using the Octet Data Analysis software.

### Cross-Reactivity of Candidate Antibodies by ELISA

Nunc MaxiSorp™ flat-bottom 96-well plates were coated with human or cyno uPAR (3.19 μg/mL) at 4° C. overnight, and plates were washed with wash buffer and blocked with 5% non-fat dry milk. A standard log serial dilution of each antibody candidate was added to the uPAR-coated plates and incubated at 4° C. overnight. Plates were washed three times and incubated with 50 μL of HRP-conjugated goat anti-human (H+L) antibody (Biorad). After a two-hr incubation, plates were washed, and 100 μL of 1-Step™ Turbo TMB-ELISA Substrate Solution (Thermo Scientific) was added to each well. The reaction was quenched with 2M H₂SO₄ for 5 min at room temperature, and the optical density of each well was measured at 450 nm using a SpectraMax190 microplate reader. The resulting dose-response curves were used to determine the minimum dose of antibodies required to achieve 50% of the saturation signal and enable a quantitative comparison of binding affinity.

Recognition of Cellular uPAR by Candidate Antibodies

MDA-MB-231 cells were harvested with TrypleE and resuspended in FACS buffer (PBS+1% BSA) to 2×10⁶ cells/mL before being aliquoted to a 96-well plate (100 μL, 2×10⁵ cells/well). Cells were pelleted by centrifuging the microplate for 5 min at 400 RCF and resuspended in PBS containing serially diluted antibodies to a maximum concentration of 600 nM, followed by a 50 min incubation at 4° C. Cells were then washed three times with PBS and incubated with an AlexaFluor488-conjugated goat anti-human IgG for 50 min at 4° C. in the dark. Finally, cells were washed twice with FACS buffer and resuspended in 80 μL for FACS analysis in a Bio-rad S3e Cell Sorter.

### Inhibition of Cell Adhesion to Vitronectin

MDA-MB-231 cells were cultured in the complete medium in a humidified atmosphere of 5% CO₂ at 37° C. . . . MaxiSorp 96-wells plates were coated with vitronectin (corning) at 4° C. overnight. The wells were washed with PBS and blocked for 1 hr with 1% BSA in PBS. 50,000 MDA-MB-231 cells were seeded in each well and a serial dilution of antibody or RGDS peptide was added, and the plate was incubated at 5% CO₂ and 37° C. overnight. All wells were washed with PBS, and ice-cold methanol was added to fix cells for 10 min at room temperature. After fixation, a 5% crystal violet solution was used to stain cells. Wells were washed three times with PBS, and cells were lysed with 2% SDS lysis buffer. Each lysate was transferred to a transparent 96-well plate, and the absorption at 590 nm was recorded to determine the number of adherent cells.

### Antibody-Dependent Cellular Cytotoxicity (ADCC) In Vitro Using NK Cells

Antibody-dependent cellular cytotoxicity on MDA-MB-231 by NK cells was detected by DELFIA® EuTDA Cytotoxicity Reagents (Perkinelmer). Briefly, MDA-MB-231 cells were harvested and labeled by incubation with 2 μL/mL of the fluorescence enhancing ligand (ParkinElmerDELFIA® BATDA Labeling Reagent) for 20 min at 37° C. After the diffusion of BATDA into the cells, it was hydrolyzed and converted to 2,2′: 6′,2″-terpyridine-6,6″-dicarboxylic acid (TDA) by cytosolic acetyl esterase. Since TDA is a non-cell permeable hydrophobic ligand, it can be trapped inside the live target cells. The solution was centrifuged, and cells were washed three times with PBS. The labeled cells were reconstituted in RPMI 1640 media without phenol red and then seeded to a 96-well U-bottom sterile microplate (100 μL, 1×10⁴ cells/well). Next, 50 μL of a serial dilution of each antibody candidate was added to the assay plate and incubated at 37° C. for 5-10 min. Separately, NK-92 CD16a 176V effector cells were harvested and concentrated to approximately 1.2×10⁶ cells/mL before the addition of 50 μL to the assay plate resulting in an effector to target cell ratio of 6:1 in each well. The plate containing the antibodies, target, and effector cells was then incubated for 4 hr at 37° C. and 5% CO₂. After the incubation, the plate was centrifuged for 5 min at 400 RCF, and 25 μL of the supernatant was transferred to a flat-bottom detection plate. Finally, 200 μL of Europium solution (PerkinElmer, DELFIA® Eu-Solution) was added to each well, and the plate was incubated for 15 min at room temperature to allow the formation of a highly fluorescent stable-chelate (Eu-TDA). The resulting fluorescent signal was obtained in a time-resolved fluorimeter within 5 hr. The background death control was determined by diluting target cells with media, and the maximum death control was determined by incubating cells with 10 μL of lysis buffer (1% Triton X-100) for 30 min prior to centrifuging the plate.

Antibody-Dependent Cellular Cytotoxicity (ADCC) In Vitro Using Human PBMCs Isolated from Healthy Donors

Frozen PBMC cells were obtained commercially from AllCells. The cells were isolated from human blood by the Leuko Pak-Density gradient method, then stored in liquid nitrogen. Cells were thawed at 37° C., suspended in RPMI1640+10% FBS, and incubated at 37° C. overnight. MDA-MB-231 target cells are labeled with DELFIA BATDA in accordance with the manufacturer's instructions. Then, the effector cells PBMC cells from each donor were plated with target cells into a 96-well plate at a ratio of 50:1. The induction of ADCC was triggered upon the addition of each antibody candidate to the mix, which was incubated for 4 hours at 37° C. Finally, the supernatant was collected and mixed with Europium solution. Time-resolved fluorescent (TRF) signal intensity was used to determine the degree of cytotoxicity. Control groups are set for data normalization, including target spontaneous group (Target cells), target maximum group (Target cells lysed using Triton) and background group (Supernatant of target cell). ADCC effect is determined by the formula: Calculated ADCC was defined by the formula: % ADCC=(Sample Cytotoxicity−Target cell and Effector cell mixture spontaneous cytotoxicity)/(Target cell Maximum cytotoxicity (Triton X-100 treatment)−Target cell and Effector cell mixture spontaneous cytotoxicity)*100%. Dose-response effect is analyzed with GraphPad Prism.

### Epitope Binning

Epitope binning assay was performed using Octet RED384 in a classical sandwich assay format. All samples were prepared in assay buffer (PBS with 1% BSA), and primary antibodies were biotinylated for the immobilization on streptavidin (SA) biosensors. Each binding cycle consisted of the following steps. First, SA biosensors were dipped in assay buffer for a sensor check, and a baseline was established. Next, the biotinylated antibody was loaded on the SA biosensors. After a wash step, uPAR was bound to reach saturation. The biosensors were washed in assay buffer then moved to the next well for the association of the secondary antibody, and finally transferred to buffer-containing wells for the dissociation phase. The data analysis was performed using ForteBio Data Analysis software, and figures were made with Matlab.

### Antibody-Drug Conjugates (ADC) Cytotoxicity Screening

MDA-MB-231 cells were seeded at 2,500 cells/well in 96 well plates (Corning) at 37° C. and 5% CO₂ overnight, and cells were grew for five days in the presence of serial dilutions of antibodies ranging from 0.0032 nM to 10 nM in triplicates, combined with a Fab fragment of an anti-human IgG Fc specific antibody conjugated to monomethyl auristatin E (Fab-αHFc-CL-MMAE, Moradec) in a final concentration of 20 nM. The number of live cells was quantified by the CellTiter-Glo luminescent cell viability assay (Promega) based on luminescent detection of ATP, which is directly proportional to the number of cells present in each well. After the incubation, the luminescence was recorded using a Synergy Neo2 Multi-Mode Microplate Reader (BioTek Instruments, Inc.).

### Therapeutic Efficacy in an Orthotopic Animal Model of Human Breast Cancer

A group of 16 female Foxninu mice were orthotopically implanted with 1×10⁶ MDA-MB-231 cells and monitored for several days until tumor volume reached 75-100 mm³. Once tumor volumes were reached animals were considered eligible for therapeutic intervention starting three days after such tumor volumes were achieved. Therapeutic intervention commenced with each experimental arm receiving antibodies administered intravenously at a concentration of 30 mg/kg. A 30-day treatment regimen was conducted, with animals receiving weekly antibody treatment over a period of 30 days (Days 3, 10, 17, and 24). Animal welfare, body weight, and tumor volume were monitored continuously throughout the study. Upon the completion of the therapeutic intervention regimen tumors were harvested and prepared for histological analysis.

### Statistical Analysis

All statistical analysis were performed in GraphPad Prism version 8.0 (GraphPad Software, Inc., San Diego, CA). Dose-response curves were driven from non-linear fitting to raw values run in a minimal of three experimental replicates. Statistical analysis for all data acquisition was performed as two-way ANOVA with a post-hoc multiple-comparison Tukey test. Differences between groups were considered significant at a P value≤0.05.

### Liquid Chromatography Tandem Mass Spectrometry Analysis (LC-MS/MS)

Purified recombinant human suPAR (8 μg) was denatured with 6M urea and disulfide bonds were reduced for 20 min at 55° C. with 10 mM DTT, followed by carbamidomethylation with 12.5 mM iodoacetamide for 1 hour in the dark. Unreacted iodoacetamide was quenched with DTT and pH was balanced to pH 8, and the trypsin digestion (Promega CAT #VA9000) was carried overnight at 37° C. Samples were desalted using Pierce™ C18 Spin Tips (Thermo Scientific™, CAT #87782), dried under vacuum, and resuspended in HPLC-grade water with 0.2% TFA. LC-MS/MS analysis was performed in a LTQ Orbitrap XL mass spectrometer (Thermo) coupled to a nanoACQUITY Ultra Performance Liquid Chromatography (UPLC) System (Waters). Trypsin digestion products were separated over a Thermo ES901 C18 column and eluted with a linear gradient from 2-50% in Buffer B (acetonitrile, 0.5% formic acid). Survey scans were recorded over a 325-1500 m/z range and up to the three most-intense precursor ions (MS1 features of charge ≥2) were selected for a higher-energy collisional dissociation (HCD) at a resolution of 30 000 at m/z 200 for MS/MS [CB2]. Data from uPAR peptides were acquired using Xcalibur software and processed as previously. (Zhao et al. (2021) ACS Cent Sci. American Chemical Society 7:1638-49).

Measurements of Serum Antibody Titers in uPAR Immunized Mice

Nunc MaxiSorp™ flat-bottom 96-well plates (Invitrogen Cat #44-2404-21) were coated with human uPAR (3.19 μg/mL) at 4° C. overnight, and plates were blocked with 200 μL of blocking buffer consisting of 5% bovine serum albumin overnight at 4° C. Antibody titer determination was carried out by performing a standard log serial dilution of serum in 5% non-fat dry milk. All uPAR-coated plates were washed three times with wash buffer (50 mM Tris-HCl, 150 mM NaCl, pH 7.4+0.02% Tween 20), and each serial dilution of serum was added. The plates were incubated at 4° C. overnight and washed three times with wash buffer, and goat anti-mouse IgG (H+L)-HRP Conjugate (BioRad Cat #1706516, Dilution 1:3000) was added and incubated for two hr at room temperature in an orbital shaker. Finally, the plates were washed, and 100 μL of 1-Step™ Turbo TMB-ELISA substrate solution (Thermo Scientific, Cat #34022) was added to each well and incubated for 15 min before quenching the reaction with 2 M H₂SO₄. Plates were incubated for 5 min at room temperature and the optical density of each well was measured at 450 nM using a SpectraMax190 microplate reader against a standard curve (10-0.07 μg/mL) of mouse anti-human uPAR monoclonal antibody clone R-3 (Invitrogen Cat #MON R-3-02).

### Example 10—Humanized 11857 and 3159 Antibodies

Antibody 11857 was used as a parental template in humanization design. Based on antibody sequence analysis and homology modeling of mAb 3D structure, three humanized V_{H} (“11857 HCl”, “11857 HC2”, and “11857 HC3”) and three humanized V_{L} (“11857 LC1”, “11857 LC2”, and “11857 LC3”) sequences were designed. Sequences for the humanized 11857 antibodies are provided in Table 1, above. CDR sequences were defined using the Kabat numbering system.

In addition, antibody 3159 was used as parental template in humanization design. Based on antibody sequence analysis and homology modeling of mAb 3D structure, three humanized VH (“3159 HCl”, “3159 HC2”, and “3159 HC3”) and three humanized VL (“3159 LC1”, “3159 LC2”, and “3159 LC3”) sequences were designed. Sequences for the humanized 11857 antibodies are provided in Table 1, above. CDR sequences were defined using the Kabat numbering system.

The T20 score analyzer was used to determine the humanness score of the humanized 11857 antibodies as described in Gao et al. (2013) BMC Biotechnology. 13:55. Results are shown below in Table 3. The T20 score of humanness ranged from 84-86 (VH) and 97-99 (VK) for the humanized variable region frameworks, which was near or exceeded the threshold of “humanness” according to Gao et al., T20 scores for the humanized full-length variable regions ranged from 79-84 (VH) and 81-82 (VK). T20 scores for the humanized full-length variable heavy chain regions exceeded the threshold of humanness. Though T20 scores for full-length kappa light chain sequences were below the recommended cut-off score, based on structural modeling, maximum T20 scores were achieved without compromising the structural confirmation of the light chain.

In addition, the T20 score analyzer was used to determine the humanness score of the humanized 3159 antibodies. Results are shown below in Table 3. The T20 Analyzer score of humanness ranged from 84-86 (VH) and 97-99 (VK) for the humanized variable region frameworks, which was near or exceeded the threshold of “humanness” according to Gao et al. T20 scores for the humanized full-length variable regions ranged from 79-82 (VH) and 85-86 (VK), which were all near, or exceeding the threshold of humanness. Though T20 scores for the full-length kappa light chain sequence “3159 LC1” was just below the recommended cut-off score, based on structural modeling, maximum T20 scores were achieved without compromising the structural confirmation of the light chain.

TABLE 3
T20 Humanness Assessment for 11857 and 3159 Heavy and Light Chains

| | | | HC T20 | HC T20 | | | LC T20 | LC T20 |
|---|---|---|---|---|---|---|---|---|
| | | | Score | Score | | | Score | Score |
| | | HC | (Full | (Framework | Sample | LC | (Full | (Framework |
| ID | Species | Framework | Length) | only) | ID | Framework | Length) | only) |
| 11857 | Mus | IGHV1-26 | 67.8 | 67.2 | 11857 | IGKV10-96 | 70.1 | 75.8 |
| Parental | Musculus | | | | Parental | | | |
| HC | | | | | LC | | | |
| 11857 | Homo | IGHV1-69 | 79.1 | 84 | 11857 | IGKV1-9 | 80.8 | 89.4 |
| HC1 | sapiens | | | | LC1 | | | |
| 11857 | Homo | IGHV1-3 | 82.6 | 88.3 | 11857 | IGK1-16 | 81.1 | 89.8 |
| HC2 | sapiens | | | | LC2 | | | |
| 11857 | Homo | IGHV1-2 | 83.9 | 86.2 | 11857 | IGKV1-27 | 82 | 90.6 |
| HC3 | sapiens | | | | LC3 | | | |
| 3159 | Mus | IGHV8-12 | 64.4 | 63.3 | 3159 | IGKV1-117 | 75.3 | 84.2 |
| Parental | Musculus | | | | Parental | | | |
| HC | | | | | LC | | | |
| 3159 | Homo | IGHV3-11 | 80.2 | 86 | 3159 | IGKV3D-11 | 84.8 | 96.6 |
| HC1 | sapiens | | | | LC1 | | | |
| 3159 | Homo | IGHV3-21 | 79.2 | 84.2 | 3159 | IGKV3-11 | 85.9 | 97.6 |
| HC2 | sapiens | | | | LC2 | | | |

Humanized 11857 and 3159 antibodies were assayed for binding to human and cyno uPAR by biolayer interferometry (BLI). The BLI curves were provided and their affinity (K_{D}) values are reported in Table 4. Kinetic constant ranges for Octet HTX were between 1 mM to 10 pM. Thus, a calculated KD lower than 10 pM is to be interpreted as KD<10 pM. For two antibodies (11857 HC2+LC3 and 11857 HC3+LC3), production yields were insufficient to perform kinetics analysis.

Two breast cancer cell lines MDA-MB-231 (high uPAR expression) and MCF-7 (low uPAR expression) were used to validate cell surface uPAR binding by a flow cytometry-based cellular assay for humanized and parental 11857 or 3159. Cells were incubated with humanized and parental 11857 or 3159, and stained with APC-conjugated anti-human Fc antibody. MFI comparison at 10 μg/mL showed specific binding on MDA-MB-231 and low nonspecific binding on MCF7 of the testing articles.

Six humanized 11857 variants and the parental chimera were assayed by flow cytometry-based cellular assay for EC50 determination using MDA-MB-231 cell line, and EC50 values are between 0.2119 to 1.206 (μg/mL). For three antibodies (11857 HC2+LC3, 11857 HC3+LC2 and 11857 HC3+LC3), production yields were insufficient to perform EC50 assay. In addition, Nine humanized 3159 variants and the parental chimera were assayed by flow cytometry-based cellular assay for EC50 determination using MDA-MB-231 cell line, and EC50 values are between 0.1852 to 0.5176 (μg/mL).

TABLE 4
Kinetics analysis results for parental and humanized 11857 and 3159

| Sample ID | Human uPAR K_{D} (M) | | Cyno uPAR K_{D} (M) | FACS EC₅₀ (μg/mL) |
|---|---|---|---|---|
| 11857 Parental | 6.37E−09 | | 8.48E−09 | 0.545 |
| 11857 HC1 + LC1 | 1.27E−07 | | 6.97E−08 | 0.6947 |
| 11857 HC1 + LC2 | 5.34E−08 | | 3.24E−08 | 0.2228 |
| 11857 HC1 + LC3 | n.d. | | n.d. | 1.206 |
| 11857 HC2 + LC1 | 1.25E−07 | | 3.07E−07 | 0.4323 |
| 11857 HC2 + LC2 | 4.57E−08 | | 5.95E−08 | 0.2119 |
| 11857 HC2 + LC3 | n/a | | n/a | n/a |
| 11857 HC3 + LC1 | 1.47E−07 | | 8.39E−08 | 0.3037 |
| 11857 HC3 + LC2 | 7.56E−08 | | 5.96E−08 | n/a |
| 11857 HC3 + LC3 | n/a | | n/a | n/a |
| 3159 Parental | <10 | pM | 9.34E−07 | 0.1884 |
| 3159 HC1 + LC1 | 6.79E−09 | | 5.56E−07 | 0.1869 |
| 3159 HC1 + LC2 | <10 | pM | 5.87E−06 | 0.5176 |
| 3159 HC1 + LC3 | 5.579E−09 | | 1.62E−07 | 0.2702 |
| 3159 HC2 + LC1 | <10 | pM | 4.11E−08 | 0.3794 |
| 3159 HC2 + LC2 | <10 | pM | 4.04E−07 | 0.3196 |
| 3159 HC2 + LC3 | <10 | pM | 2.55E−07 | 0.2052 |
| 3159 HC3 + LC1 | <10 | pM | 2.02E−07 | 0.488 |

| | | | |
|---|---|---|---|
| 3159 HC3 + LC2 | 4.655E−09 | 2.24E−06 | 0.2141 |
| 3159 HC3 + LC3 | 7.389E−09 | 5.85E−08 | 0.1852 |
| 11857 Parental | 6.37E−09 | 8.48E−09 | 0.545 |

Also, uPAR expression level in MDA-MB-231 and UMUC3 cell lines was compared by flow cytometer. Cells were incubated with parental 11857 and stained with PE-conjugated anti-human Fc antibody. The result identified uPAR expression level is about 3-fold lower in UMUC3 compared to MDA-MB-231 cells.

### Example 11—Imaging Studies

5 mCi of Zr-89 was labeled with 500 μg of desferrioxamine (DFO)-conjugated 3159 and 11857 antibodies described above with respect to Example 10. In the present example, 11857 and 3159 refer to 11857 HC2+LC2 and 3159 HC2+LC2, respectively. A quality control analysis showed 100% labeling, and a yield of 95%. Imaging was conducted with a UMUC3 tumor model in nude male mice. N=4 for Zr89-3159, and N=3 for Zr89-11857. Doses were administered at 200-280 μCi per mouse. PET/CT images were taken at 30 min, 4 hr, 19 hr, 24 hr, 48 hr, 72 hr, 96 hr and 120 hr post-administration.

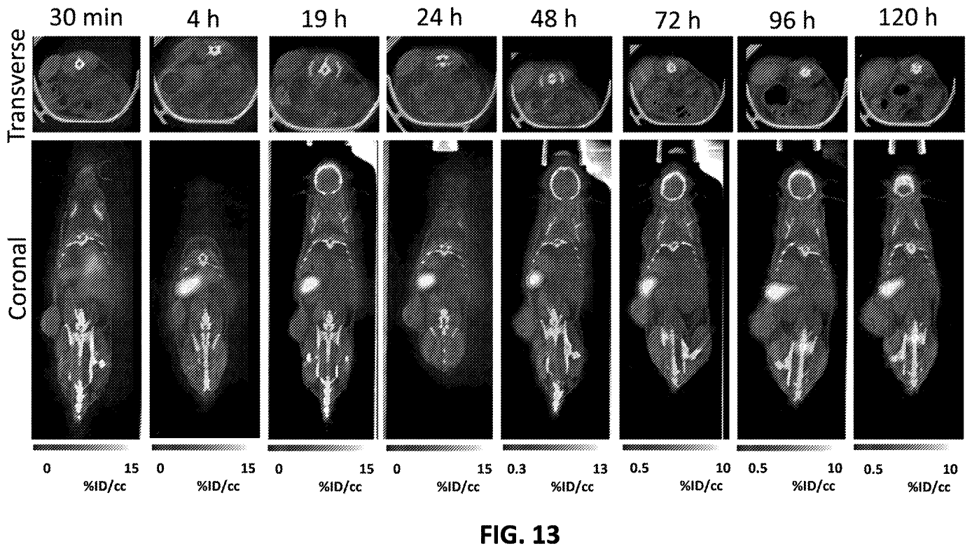

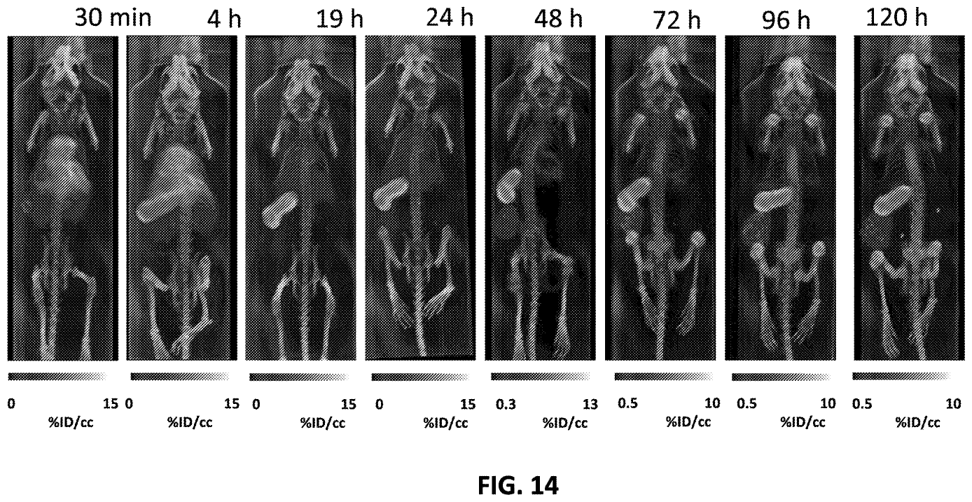

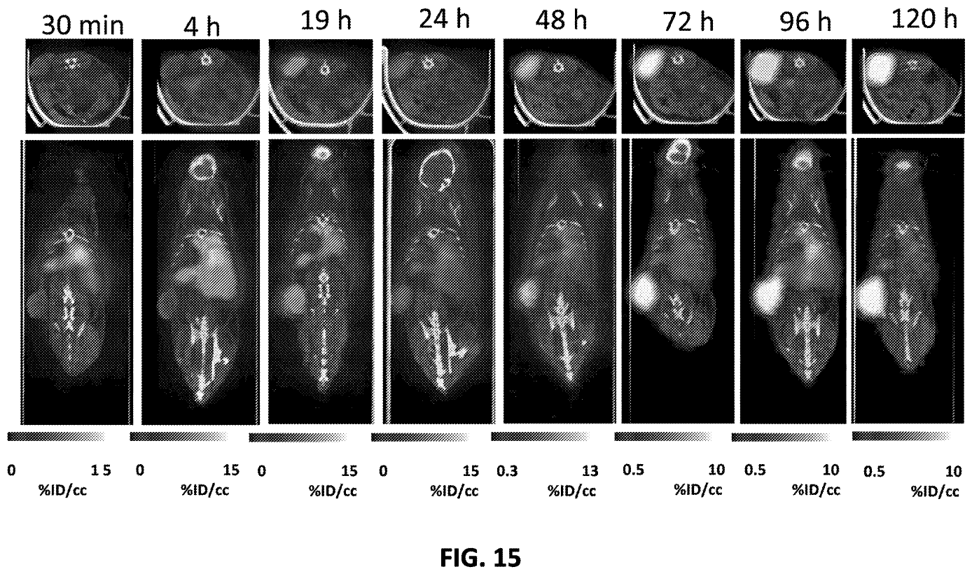

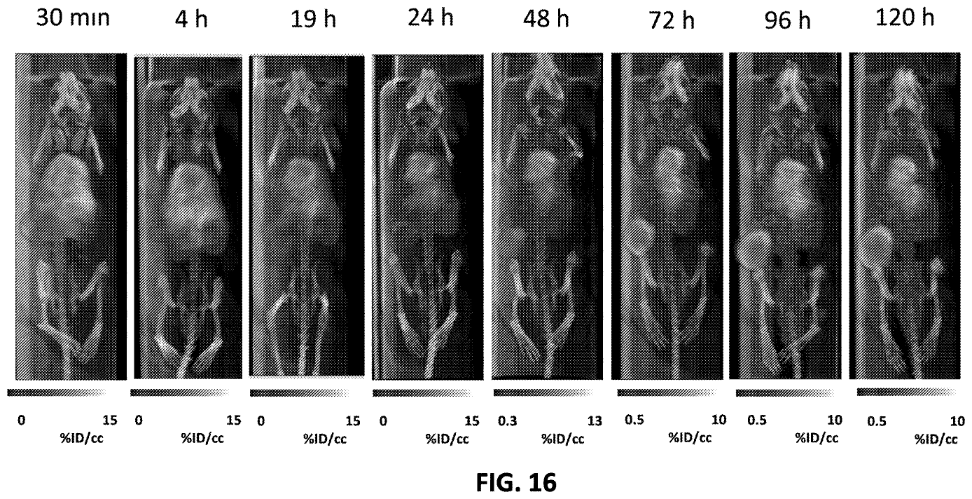

Representative transaxial and coronal positron emission tomography-computed tomography (PET/CT) slices acquired from male nu/nu mice bearing subcutaneous UMUC3 xenografts are shown in FIG. 13. The mice received ˜250 μCi of 89Zr-3159 IgG and were imaged at the indicated time point. The position of the tumor is indicated with an arrow. Representative maximum intensity projections acquired from male nu/nu mice bearing subcutaneous UMUC3 xenografts are shown in FIG. 14. The mice received ˜250 μCi of 89Zr-3159 IgG and were imaged at the indicated time point. The position of the tumor is indicated with an arrow. Representative transaxial and coronal PET/CT slices acquired from male nu/nu mice bearing subcutaneous UMUC3 xenografts are shown in FIG. 15. The mice received ˜250 μCi of 89Zr-11857 IgG and were imaged at the indicated time point. The position of the tumor is indicated with an arrow. Representative maximum intensity projections acquired from male nu/nu mice bearing subcutaneous UMUC3 xenografts are shown in FIG. 16. The mice received ˜250 μCi of ⁸⁹Zr-11857 IgG and were imaged at the indicated time point. The position of the tumor is indicated with an arrow.

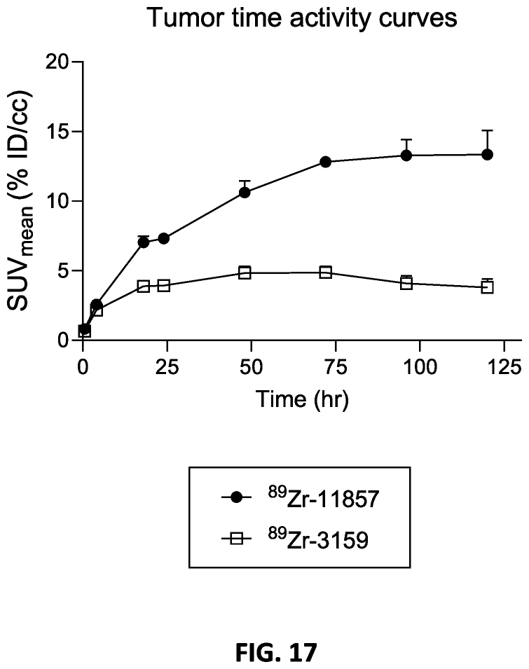

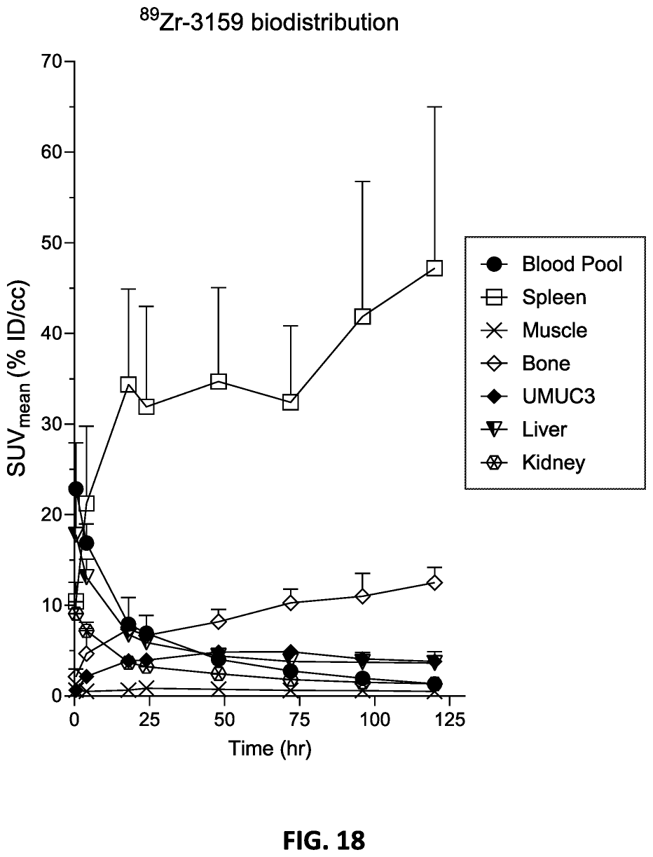

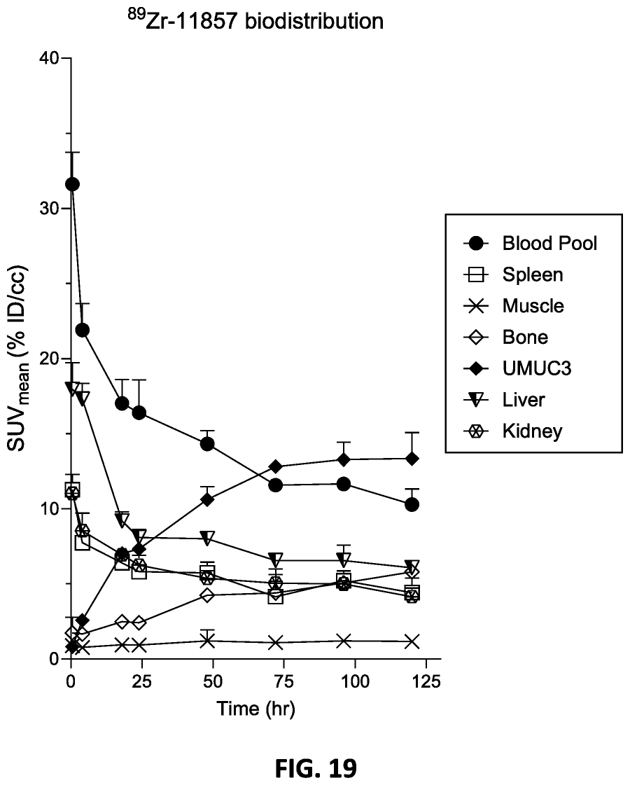

SUVmean data (FIG. 17) was acquired by region of interest analysis on the tumors from the mice in the imaging cohorts. The data was expressed as mean with standard deviation. Data were acquired from 4 tumors in the 3159 cohort and 3 tumors in the 11857 cohort. In addition, SUVmean data was acquired by region of interest analysis on the tumor and various normal tissues from the mice in the imaging cohorts. The data is expressed as mean with standard deviation. Data were acquired from 4 tumors in the 3159 cohort (FIG. 18) and 3 tumors in the 11857 cohort (FIG. 19).

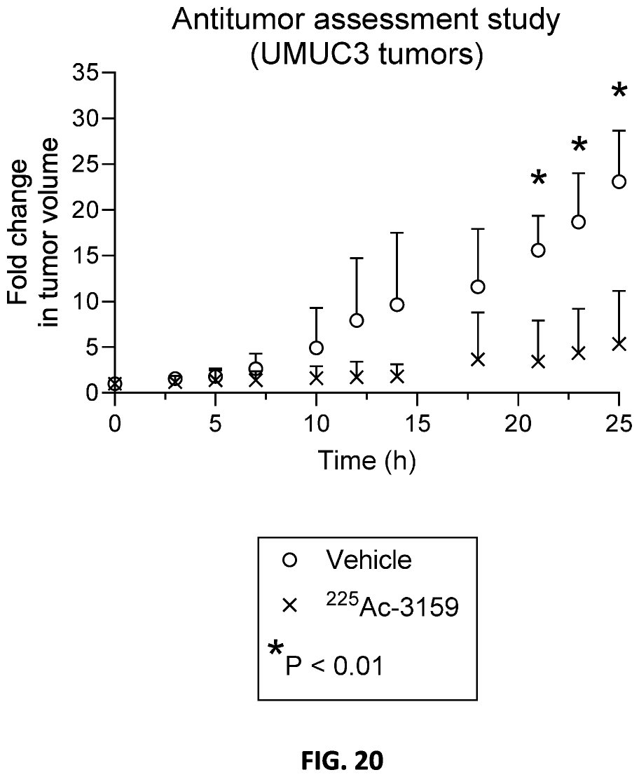

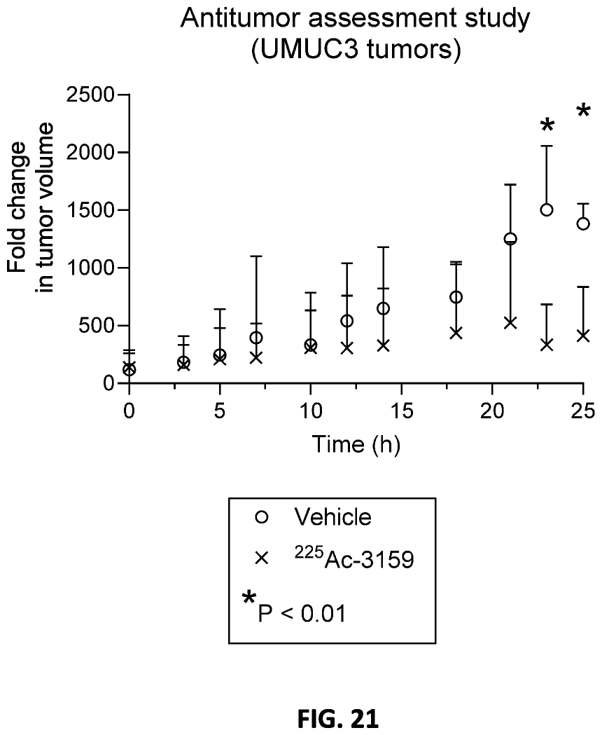

Fold change in volume (normalized to volume at day 0) of UMUC3 tumors treated with 225Ac-labeled 3159 is depicted in FIG. 20. To enable radiolabeling, 3159 was coupled to NHS-Macropa through lysine residues. The radiopharmaceutical was administered at 0.8 μCi per mouse on day 0 via tail vein. Tumor volume measurements were recorded for the vehicle (n=10) and drug treated (n=18) arms. Volume change of the of UMUC3 tumors treated with ²²⁵Ac-labeled 3159 is shown in FIG. 21.

### REFERENCES

1. Smith H W, Marshall C J. Regulation of cell signalling by uPAR. Nat Rev Mol Cell Biol. 2010; 11:23-36.
2. Lund I K, Illemann M, Thurison T, Christensen I J, Høyer-Hansen G. uPAR as anti-cancer target: evaluation of biomarker potential, histological localization, and antibody-based therapy. Curr Drug Targets. 2011; 12:1744-60.
3. Mazar A P, Ahn R W, O'Halloran T V. Development of Novel Therapeutics Targeting the Urokinase Plasminogen Activator Receptor (uPAR) and Their Translation Toward the Clinic. Curr Pharm Des. 17:1970-8.
4. Stroomberg H V, Kristensen G, Drimer Berg K, Lippert S, Brasso K, Røder MA. The Association between Plasma Levels of Intact and Cleaved uPAR Levels and the Risk of Biochemical Recurrence after Radical Prostatectomy for Prostate Cancer. Diagnostics. 2020; 10:877.
5. Liu K L, Fan J H, Wu J. Prognostic Role of Circulating Soluble uPAR in Various Cancers: a Systematic Review and Meta-Analysis. Clin Lab. 2017; 63:871-80.
6. de Bock C E, Wang Y. Clinical significance of urokinase-type plasminogen activator receptor (uPAR) expression in cancer. Med Res Rev. 2004; 24:13-39.
7. Yuan C, Guo Z, Yu S, Jiang L, Huang M. Development of inhibitors for uPAR: blocking the interaction of uPAR with its partners. Drug Discov Today. 2021; 26:1076-85.
8. Meijer-van Gelder M E, Look M P, Peters H A, Schmitt M, Brunner N, Harbeck N, et al. Urokinase-type plasminogen activator system in breast cancer: association with tamoxifen therapy in recurrent disease. Cancer Res. 2004; 64:4563-8.
9. Bianchini G, Balko J M, Mayer I A, Sanders M E, Gianni L. Triple-negative breast cancer: challenges and opportunities of a heterogeneous disease. Nat Rev Clin Oncol. 2016; 13:674-90.
10. Konecny G, Untch M, Arboleda J, Wilson C, Kahlert S, Boettcher B, et al. Her-2/neu and urokinase-type plasminogen activator and its inhibitor in breast cancer. Clin Cancer Res Off J Am Assoc Cancer Res. 2001; 7:2448-57.
11. Jo M, Lester R D, Montel V, Eastman B, Takimoto S, Gonias S L. Reversibility of Epithelial-Mesenchymal Transition (EMT) Induced in Breast Cancer Cells by Activation of Urokinase Receptor-dependent Cell Signaling. J Biol Chem. 2009; 284:22825-33.
12. Chandran V I, Eppenberger-Castori S, Venkatesh T, Vine K L, Ranson M. HER2 and uPAR cooperativity contribute to metastatic phenotype of HER2-positive breast cancer.

### Oncoscience. 2015; 2:207-24.

13. Uhr J. uPAR and HER2 Genes Are Usually Co-Amplified in Individual Breast Cancer Cells from Blood and Tissues. Breast Care. 2008; 3:16-9.
14. Li C, Cao S, Liu Z, Ye X, Chen L, Meng S. RNAi-mediated downregulation of uPAR synergizes with targeting of HER2 through the ERK pathway in breast cancer cells. Int J Cancer. 2010; 127:1507-16.
15. Gajria D, Chandarlapaty S. HER2-amplified breast cancer: mechanisms of trastuzumab resistance and novel targeted therapies. Expert Rev Anticancer Ther. 2011; 11:263-75.
16. Pohlmann P R, Mayer I A, Mernaugh R. Resistance to Trastuzumab in Breast Cancer. Clin Cancer Res. 2009; 15:7479-91.
17. LeBeau A M, Duriseti S, Murphy S T, Pepin F, Hann B, Gray J W, et al. Targeting uPAR with Antagonistic Recombinant Human Antibodies in Aggressive Breast Cancer. Cancer Res. 2013; 73:2070-81.
18. Zhao B, Gandhi S, Yuan C, Luo Z, Li R, Gårdsvoll H, et al. Stabilizing a flexible interdomain hinge region harboring the SMB binding site drives uPAR into its closed conformation. J Mol Biol. 2015; 427:1389-403.
19. Rabbani S A, Ateeq B, Arakelian A, Valentino M L, Shaw D E, Dauffenbach L M, et al. An Anti-Urokinase Plasminogen Activator Receptor Antibody (ATN-658) Blocks Prostate Cancer Invasion, Migration, Growth, and Experimental Skeletal Metastasis In Vitro and In Vivo. Neoplasia. 2010; 12:778-88.
20. Rullo A F, Fitzgerald K J, Muthusamy V, Liu M, Yuan C, Huang M, et al. Re-engineering the Immune Response to Metastatic Cancer: Antibody-Recruiting Small Molecules Targeting the Urokinase Receptor. Angew Chem Int Ed. 2016; 55:3642-6.
21. Mani T, Wang F, Knabe W E, Sinn A L, Khanna M, Jo I, et al. Small-molecule inhibition of the uPAR.uPA interaction: Synthesis, biochemical, cellular, in vivo pharmacokinetics and efficacy studies in breast cancer metastasis. Bioorg Med Chem. 2013; 21:2145-55.
22. Minopoli M, Polo A, Ragone C, Ingangi V, Ciliberto G, Pessi A, et al. Structure-function relationship of an Urokinase Receptor-derived peptide which inhibits the Formyl Peptide Receptor type 1 activity. Sci Rep. Nature Publishing Group; 2019; 9:12169.
23. Kenny H A, Leonhardt P, Ladanyi A, Yamada S D, Montag A, Im H K, et al. Targeting the Urokinase Plasminogen Activator Receptor Inhibits Ovarian Cancer Metastasis. Clin Cancer Res Off J Am Assoc Cancer Res. 2011; 17:459-71.
24. Harel E T, Drake P M, Barfield R M, Lui I, Farr-Jones S, Van′t Veer L, et al. Antibody-Drug Conjugates Targeting the Urokinase Receptor (uPAR) as a Possible Treatment of Aggressive Breast Cancer. Antibodies Basel Switz. 2019; 8:E54.
25. Kriegbaum M C, Persson M, Haldager L, Alpizar-Alpizar W, Jacobsen B, Gårdsvoll H, et al. Rational targeting of the urokinase receptor (uPAR): development of antagonists and non-invasive imaging probes. Curr Drug Targets. 2011; 12:1711-28.
26. Baart V M, Houvast R D, de Geus-Oei L F, Quax P H A, Kuppen P J K, Vahrmeijer A L, et al. Molecular imaging of the urokinase plasminogen activator receptor: opportunities beyond cancer. EJNMMI Res. 2020; 10:87.
27. Duriseti S, Goetz D H, Hostetter D R, LeBeau A M, Wei Y, Craik C S. Antagonistic anti-urokinase plasminogen activator receptor (uPAR) antibodies significantly inhibit uPAR-mediated cellular signaling and migration. J Biol Chem. 2010; 285:26878-88.
28. Betts A, Keunecke A, van Steeg T J, van der Graaf P H, Avery L B, Jones H, et al. Linear pharmacokinetic parameters for monoclonal antibodies are similar within a species and across different pharmacological targets: A comparison between human, cynomolgus monkey and hFcRn Tg32 transgenic mouse using a population-modeling approach. mAbs. 2018; 10:751-64.
29. Iwasaki K, Uno Y, Utoh M, Yamazaki H. Importance of cynomolgus monkeys in development of monoclonal antibody drugs. Drug Metab Pharmacokinet. 2019; 34:55-63.
30. Winters A, McFadden K, Bergen J, Landas J, Berry K A, Gonzalez A, et al. Rapid single B cell antibody discovery using nanopens and structured light. mAbs. 2019; 11:1025-35.
31. Pedrioli A, Oxenius A. Single B cell technologies for monoclonal antibody discovery. Trends Immunol. Elsevier; 2021; 42:1143-58.
32. Yeku O, Frohman M A. Rapid amplification of cDNA ends (RACE). Methods Mol Biol Clifton N J. 2011; 703:107-22.
33. Arnould L, Gelly M, Penault-Llorca F, Benoit L, Bonnetain F, Migeon C, et al. Trastuzumab-based treatment of HER2-positive breast cancer: an antibody-dependent cellular cytotoxicity mechanism? Br J Cancer. Nature Publishing Group; 2006; 94:259-67.
34. Kang T H, Jung S T. Boosting therapeutic potency of antibodies by taming Fc domain functions. Exp Mol Med. 2019; 51:1-9.
35. Petricevic B, Laengle J, Singer J, Sachet M, Fazekas J. Steger G, et al. Trastuzumab mediates antibody-dependent cell-mediated cytotoxicity and phagocytosis to the same extent in both adjuvant and metastatic HER2/neu breast cancer patients. J Transl Med. 2013; 11:307.
36. Forsström B, Bisławska Axnäs B, Rockberg J, Danielsson H, Bohlin A, Uhlen M. Dissecting Antibodies with Regards to Linear and Conformational Epitopes. PLOS ONE. 2015; 10:e0121673.
37. McKeage K, Perry C M. Trastuzumab: a review of its use in the treatment of metastatic breast cancer overexpressing HER2. Drugs. 2002; 62:209-43.
38. Mazzotta M, Krasniqi E, Barchiesi G, Pizzuti L, Tomao F, Barba M, et al. Long-Term Safety and Real-World Effectiveness of Trastuzumab in Breast Cancer. J Clin Med. 2019; 8:254.
39. Boyerinas B, Jochems C, Fantini M, Heery C R, Gulley J L, Tsang K Y, et al. Antibody-dependent cellular cytotoxicity (ADCC) activity of a novel anti-PD-L1 antibody avelumab (MSB0010718C) on human tumor cells. Cancer Immunol Res. 2015; 3:1148-57.
40. Dean A Q, Luo S, Twomey J D, Zhang B. Targeting cancer with antibody-drug conjugates: Promises and challenges. mAbs. 2021; 13:1951427.
41. Ferraris G M S, Schulte C, Buttiglione V, De Lorenzi V, Piontini A, Galluzzi M, et al. The interaction between uPAR and vitronectin triggers ligand-independent adhesion signalling by integrins. EMBO J. 2014; 33:2458-72.
42. LeBeau A M, Sevillano N, King M L, Duriseti S, Murphy S T, Craik C S, et al. Imaging the Urokinase Plasminongen Activator Receptor in Preclinical Breast Cancer Models of Acquired Drug Resistance. Theranostics. 2014; 4:267-79.
43. Appella E, Robinson E A, Ullrich S J, Stoppelli M P, Corti A, Cassani G, et al. The receptor-binding sequence of urokinase. A biological function for the growth-factor module of proteases. J Biol Chem. 1987; 262:4437-40.
44. Estreicher A, Wohlwend A, Belin D, Schleuning W D, Vassalli J D. Characterization of the Cellular Binding Site for the Urokinase-type Plasminogen Activator. J Biol Chem. Elsevier; 1989; 264:1180-9.
45. Zhai B-T, Tian H, Sun J, Zou J-B, Zhang X-F, Cheng J-X, et al. Urokinase-type plasminogen activator receptor (uPAR) as a therapeutic target in cancer. J Transl Med. 2022; 20:135.
46. Mahmood N, Mihalcioiu C, Rabbani S A. Multifaceted Role of the Urokinase-Type Plasminogen Activator (uPA) and Its Receptor (uPAR): Diagnostic, Prognostic, and Therapeutic Applications. Front Oncol. 2018; 8:24.
47. Mahmood N, Arakelian A, Khan H A, Tanvir I, Mazar A P, Rabbani S A. uPAR antibody (huATN-658) and Zometa reduce breast cancer growth and skeletal lesions. Bone Res. 2020; 8:18.
48. Han C, Gunn G R, Marini J C, Shankar G, Han Hsu H, Davis H M. Pharmacokinetics and immunogenicity investigation of a human anti-interleukin-17 monoclonal antibody in non-naïve cynomolgus monkeys. Drug Metab Dispos Biol Fate Chem. 2015; 43:762-70.
49. Derebe M G, Nanjunda R K, Gilliland G L, Lacy E R, Chiu M L. Human IgG subclass cross-species reactivity to mouse and cynomolgus monkey Fcγ receptors. Immunol Lett. 2018; 197:1-8.
50. Zost S J, Gilchuk P, Chen R E, Case J B, Reidy J X, Trivette A, et al. Rapid isolation and profiling of a diverse panel of human monoclonal antibodies targeting the SARS-COV-2 spike protein. Nat Med. 2020; 26:1422-7.
51. Dixon K J, Wu J, Walcheck B. Engineering Anti-Tumor Monoclonal Antibodies and Fc Receptors to Enhance ADCC by Human NK Cells. Cancers. 2021; 13:312.
52. St-Pierre F, Bhatia S, Chandra S. Harnessing Natural Killer Cells in Cancer Immunotherapy: A Review of Mechanisms and Novel Therapies. Cancers. 2021; 13:1988.
53. Yogo R, Yamaguchi Y, Watanabe H, Yagi H, Satoh T, Nakanishi M, et al. The Fab portion of immunoglobulin G contributes to its binding to Fcγ receptor III. Sci Rep. 2019; 9:11957.
54. Wang W, Chen Q. Antigen improves binding of IgGs to FcγRs in SPR analysis. Anal Biochem. 2022; 640:114411.
55. Sun Y, Izadi S, Callahan M, Deperalta G, Wecksler A T. Antibody-receptor interactions mediate antibody-dependent cellular cytotoxicity. J Biol Chem. 2021; 297:100826.
56. Acharya P, Tolbert W D, Gohain N, Wu X, Yu L, Liu T, et al. Structural definition of an antibody-dependent cellular cytotoxicity response implicated in reduced risk for HIV-1 infection. J Virol. 2014; 88:12895-906.
57. Tolbert W D, Sherburn R T, Van V, Pazgier M. Structural Basis for Epitopes in the gp120 Cluster A Region that Invokes Potent Effector Cell Activity. Viruses. 2019; 11:69.
58. Mielke D, Bandawe G, Pollara J. Abrahams M-R, Nyanhete T, Moore P L, et al. Antibody-Dependent Cellular Cytotoxicity (ADCC)-Mediating Antibodies Constrain Neutralizing Antibody Escape Pathway. Front Immunol. 2019; 10:2875.
59. Kohrt H E, Houot R, Marabelle A, Cho H J, Osman K, Goldstein M, et al. Combination strategies to enhance antitumor ADCC. Immunotherapy. 2012; 4:511-27.
60. Pirazzoli V, Ferraris G M S, Sidenius N. Direct evidence of the importance of vitronectin and its interaction with the urokinase receptor in tumor growth. Blood. 2013; 121:2316-23.
61. Wei Y, Waltz D A, Rao N, Drummond R J, Rosenberg S, Chapman H A. Identification of the urokinase receptor as an adhesion receptor for vitronectin. J Biol Chem. 1994; 269:32380-8.
62. Deng G, Curriden S A, Wang S, Rosenberg S, Loskutoff D J. Is plasminogen activator inhibitor-1 the molecular switch that governs urokinase receptor-mediated cell adhesion and release? J Cell Biol. 1996; 134:1563-71.
63. Deng G, Curriden S A, Hu G, Czekay R P, Loskutoff D J. Plasminogen activator inhibitor-1 regulates cell adhesion by binding to the somatomedin B domain of vitronectin. J Cell Physiol. 2001; 189:23-33.
64. Madsen C D, Ferraris G M S, Andolfo A, Cunningham O, Sidenius N. uPAR-induced cell adhesion and migration: vitronectin provides the key. J Cell Biol. 2007; 177:927-39.
65. Ferraro E, Drago J Z, Modi S. Implementing antibody-drug conjugates (ADCs) in HER2-positive breast cancer: state of the art and future directions. Breast Cancer Res. 2021; 23:84.
66. Vilhardt F, Nielsen M, Sandvig K, van Deurs B. Urokinase-Type Plasminogen Activator Receptor Is Internalized by Different Mechanisms in Polarized and Nonpolarized Madin-Darby Canine Kidney Epithelial Cells. Mol Biol Cell. 1999; 10:179-95.
67. Cortese K, Sahores M, Madsen C D, Tacchetti C, Blasi F. Clathrin and LRP-1-independent constitutive endocytosis and recycling of uPAR. PloS One. 2008; 3: e3730.
68. Noh H, Hong S, Huang S. Role of urokinase receptor in tumor progression and development. Theranostics. 2013; 3:487-95.
69. Wilmore J R, Jones D D, Allman D. Improved resolution of plasma cell subpopulations by flow cytometry. Eur J Immunol. 2017; 47:1386-8.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. An antibody that specifically binds to human urokinase-type plasminogen activator receptor (uPAR) and competes for binding to uPAR with an antibody comprising:
a variable heavy chain (V_{H}) polypeptide comprising
a V_{H} CDR1 comprising the amino acid sequence TSGMGVS (SEQ ID NO:2),
a V_{H} CDR2 comprising the amino acid sequence HIYWDDDKRYNPSLKT (SEQ ID NO: 3), and
a V_{H} CDR3 comprising the amino acid sequence RVRNYFSGTSYWYFDV (SEQ ID NO: 4); and
a variable light chain (V_{L}) polypeptide comprising
a V_{L} CDR1 comprising the amino acid sequence RSSQNILHRTGNTYLE (SEQ ID NO: 6),
a V_{L} CDR2 comprising the amino acid sequence KVSNRFS (SEQ ID NO:7), and
a V_{L} CDR3 comprising the amino acid sequence FQGSYVPFT (SEQ ID NO:8);
a variable heavy chain (V_{H}) polypeptide comprising
a V_{H} CDR1 comprising the amino acid sequence SHDMS (SEQ ID NO:10),
a V_{H} CDR2 comprising the amino acid sequence AIDSDGGLTYYSNSRER (SEQ ID NO: 11), and
a V_{H} CDR3 comprising the amino acid sequence RRASYWYFDV (SEQ ID NO:12); and
a variable light chain (V_{L}) polypeptide comprising
a V_{L} CDR1 comprising the amino acid sequence RASQNIGTSIH (SEQ ID NO:14),
a V_{L} CDR2 comprising the amino acid sequence YASESIS (SEQ ID NO:15), and
a V_{L} CDR3 comprising the amino acid sequence QQSNSWPT (SEQ ID NO:16);
a variable heavy chain (VA) polypeptide comprising
a V_{H} CDR1 comprising the amino acid sequence DYYMN (SEQ ID NO:18),
a V_{H} CDR2 comprising the amino acid sequence NINPNNGGTDYNQKFKG (SEQ ID NO: 19), and
a V_{H} CDR3 comprising the amino acid sequence SYGSRFPY (SEQ ID NO:20); and
a variable light chain (V_{L}) polypeptide comprising
a V_{L} CDR1 comprising the amino acid sequence RASQDITNYLS (SEQ ID NO:22),
a V_{L} CDR2 comprising the amino acid sequence YTAVLQS (SEQ ID NO:23), and
a V_{L} CDR3 comprising the amino acid sequence QQGHTLPWT (SEQ ID NO:24); or
a variable heavy chain (VA) polypeptide comprising
a V_{H} CDR1 comprising the amino acid sequence DYYMN (SEQ ID NO:18),
a V_{H} CDR2 comprising the amino acid sequence NINPNNGGTDYNQKFKG (SEQ ID NO: 19), and
a V_{H} CDR3 comprising the amino acid sequence SYGSRFPY (SEQ ID NO:20); and
a variable light chain (V_{L}) polypeptide comprising
a V_{L} CDR1 comprising the amino acid sequence RASQDITNYLS (SEQ ID NO:22),
a V_{L} CDR2 comprising the amino acid sequence YTSFLQS (SEQ ID NO:26), and
a V_{L} CDR3 comprising the amino acid sequence QQGHTLPWT (SEQ ID NO:24).

2. The antibody of claim 1, wherein the antibody comprises:
a variable heavy chain (V_{H}) polypeptide comprising
a V_{H} CDR1 comprising the amino acid sequence TSGMGVS (SEQ ID NO:2),
a V_{H} CDR2 comprising the amino acid sequence HIYWDDDKRYNPSLKT (SEQ ID NO: 3), and
a V_{H} CDR3 comprising the amino acid sequence RVRNYFSGTSYWYFDV (SEQ ID NO: 4); and
a variable light chain (V_{L}) polypeptide comprising
a V_{L} CDR1 comprising the amino acid sequence RSSQNILHRTGNTYLE (SEQ ID NO: 6),
a V_{L} CDR2 comprising the amino acid sequence KVSNRFS (SEQ ID NO:7), and
a V_{L} CDR3 comprising the amino acid sequence FQGSYVPFT (SEQ ID NO:8);
a variable heavy chain (VA) polypeptide comprising
a V_{H} CDR1 comprising the amino acid sequence SHDMS (SEQ ID NO:10),
a V_{H} CDR2 comprising the amino acid sequence AIDSDGGLTYYSNSRER (SEQ ID NO: 11), and
a V_{H} CDR3 comprising the amino acid sequence RRASYWYFDV (SEQ ID NO:12); and
a variable light chain (V_{L}) polypeptide comprising
a V_{L} CDR1 comprising the amino acid sequence RASQNIGTSIH (SEQ ID NO:14),
a V_{L} CDR2 comprising the amino acid sequence YASESIS (SEQ ID NO:15), and
a V_{L} CDR3 comprising the amino acid sequence QQSNSWPT (SEQ ID NO:16);
a variable heavy chain (VA) polypeptide comprising
a V_{H} CDR1 comprising the amino acid sequence DYYMN (SEQ ID NO:18),
a V_{H} CDR2 comprising the amino acid sequence NINPNNGGTDYNQKFKG (SEQ ID NO: 19), and
a V_{H} CDR3 comprising the amino acid sequence SYGSRFPY (SEQ ID NO:20); and
a variable light chain (VI) polypeptide comprising
a V_{L} CDR1 comprising the amino acid sequence RASQDITNYLS (SEQ ID NO:22),
a V_{L} CDR2 comprising the amino acid sequence YTAVLQS (SEQ ID NO:23), and
a V_{L} CDR3 comprising the amino acid sequence QQGHTLPWT (SEQ ID NO:24); or
a variable heavy chain (VA) polypeptide comprising
a V_{H} CDR1 comprising the amino acid sequence DYYMN (SEQ ID NO:18),
a V_{H} CDR2 comprising the amino acid sequence NINPNNGGTDYNQKFKG (SEQ ID NO: 19), and
a V_{H} CDR3 comprising the amino acid sequence SYGSRFPY (SEQ ID NO:20); and
a variable light chain (V_{L}) polypeptide comprising
a V_{L} CDR1 comprising the amino acid sequence RASQDITNYLS (SEQ ID NO:22),
a V_{L} CDR2 comprising the amino acid sequence YTSFLQS (SEQ ID NO:26), and
a V_{L} CDR3 comprising the amino acid sequence QQGHTLPWT (SEQ ID NO:24).

3. The antibody of claim 1 or claim 2, wherein the antibody comprises:
a variable heavy chain (VA) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:1; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:5.

4. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:9; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:13.

5. The antibody of claim 1 or claim 2, wherein the antibody comprises:
a variable heavy chain (VA) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:17; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:21.

6. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:17; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:25.

7. An antibody that specifically binds to human urokinase-type plasminogen activator receptor (uPAR) and competes for binding to uPAR with an antibody comprising:
(a) a variable heavy chain (V_{H}) polypeptide comprising:
a V_{H} CDR1 comprising the amino acid sequence DYYMN (SEQ ID NO: 18); a V_{H} CDR2 comprising the amino acid sequence NINPNNGGTDYNQKFQG (SEQ ID NO: 39); and a V_{H} CDR3 comprising the amino acid sequence SYGSRFPY (SEQ ID NO: 20); or
a V_{H} CDR1 comprising the amino acid sequence DYYMN (SEQ ID NO: 18); a V_{H} CDR2 comprising the amino acid sequence NINPNNGGTDYSQKFQG (SEQ ID NO: 37); and a V_{H} CDR3 comprising the amino acid sequence SYGSRFPY (SEQ ID NO: 20); and
a variable light chain (V_{L}) polypeptide comprising:
a V_{L} CDR1 comprising the amino acid sequence RASQDITNYLS (SEQ ID NO:22); a V_{L} CDR2 comprising the amino acid sequence YTAVLQS (SEQ ID NO:23); and a V_{L} CDR3 comprising the amino acid sequence QQGHTLPWT (SEQ ID NO: 24); or
(b) a variable heavy chain (V_{H}) polypeptide comprising:
a V_{H} CDR1 comprising the amino acid sequence TSGMGVS (SEQ ID NO: 2); a V_{H} CDR2 comprising the amino acid sequence HIYWDDDKRYSTSLKT (SEQ ID NO: 44); and a V_{H} CDR3 comprising the amino acid sequence RVRNYFSGTSYWYFDV (SEQ ID NO:4); or
a V_{H} CDR1 comprising the amino acid sequence TSGMGVS (SEQ ID NO: 2); a V_{H} CDR2 comprising the amino acid sequence HIYWDDDKRYSPSLKS (SEQ ID NO: 46); and a V_{H} CDR3 comprising the amino acid sequence RVRNYFSGTSYWYFDV (SEQ ID NO:4); or
a V_{H} CDR1 comprising the amino acid sequence TSGMGVS (SEQ ID NO: 2); a V_{H} CDR2 comprising the amino acid sequence HIYWDDDKRYNPSLKS (SEQ ID NO: 48); and a V_{H} CDR3 comprising the amino acid sequence RVRNYFSGTSYWYFDV (SEQ ID NO:4); and
a variable light chain (V_{L}) polypeptide comprising:
a V_{L} CDR1 comprising the amino acid sequence KSSQNILHRTGNTYLE (SEQ ID NO: 50); a V_{L} CDR2 comprising the amino acid sequence KVSNRFS (SEQ ID NO: 7); and a V_{L} CDR3 comprising the amino acid sequence FQGSYVPFT (SEQ ID NO: 8); or
a V_{L} CDR1 comprising the amino acid sequence RSSQNILHRTGNTYLE (SEQ ID NO: 6); a V_{L} CDR2 comprising the amino acid sequence KVSNRFS (SEQ ID NO: 7); and a V_{L} CDR3 comprising the amino acid sequence FQGSYVPFT (SEQ ID NO: 8); or
a V_{L} CDR1 comprising the amino acid sequence RSSQNILHRTGNTYLD (SEQ ID NO: 53); a V_{L} CDR2 comprising the amino acid sequence KVSNRFS (SEQ ID NO: 7); and a V_{L} CDR3 comprising the amino acid sequence FQGSYVPFT (SEQ ID NO: 8).

8. The antibody of claim 7, wherein the antibody comprises:
(a) a variable heavy chain (VA) polypeptide comprising:
a V_{H} CDR1 comprising the amino acid sequence DYYMN (SEQ ID NO:18); a V_{H} CDR2 comprising the amino acid sequence NINPNNGGTDYNQKFQG (SEQ ID NO: 39); and a V_{H} CDR3 comprising the amino acid sequence SYGSRFPY (SEQ ID NO: 20); or
a V_{H} CDR1 comprising the amino acid sequence DYYMN (SEQ ID NO:18); a V_{H} CDR2 comprising the amino acid sequence NINPNNGGTDYSQKFQG (SEQ ID NO: 37); and a V_{H} CDR3 comprising the amino acid sequence SYGSRFPY (SEQ ID NO: 20); and
a variable light chain (V_{L}) polypeptide comprising:
a V_{L} CDR1 comprising the amino acid sequence RASQDITNYLS (SEQ ID NO:22); a V_{L} CDR2 comprising the amino acid sequence YTAVLQS (SEQ ID NO:23); and a V_{L} CDR3 comprising the amino acid sequence QQGHTLPWT (SEQ ID NO: 24); or
(b) a variable heavy chain (VA) polypeptide comprising:
a V_{H} CDR1 comprising the amino acid sequence TSGMGVS (SEQ ID NO: 2); a V_{H} CDR2 comprising the amino acid sequence HIYWDDDKRYSTSLKT (SEQ ID NO: 44); and a V_{H} CDR3 comprising the amino acid sequence RVRNYFSGTSYWYFDV (SEQ ID NO:4); or
a V_{H} CDR1 comprising the amino acid sequence TSGMGVS (SEQ ID NO: 2); a V_{H} CDR2 comprising the amino acid sequence HIYWDDDKRYSPSLKS (SEQ ID NO: 46); and a V_{H} CDR3 comprising the amino acid sequence RVRNYFSGTSYWYFDV (SEQ ID NO:4); or
a V_{H} CDR1 comprising the amino acid sequence TSGMGVS (SEQ ID NO: 2); a V_{H} CDR2 comprising the amino acid sequence HIYWDDDKRYNPSLKS (SEQ ID NO: 48); and a V_{H} CDR3 comprising the amino acid sequence RVRNYFSGTSYWYFDV (SEQ ID NO:4); and
a variable light chain (V_{L}) polypeptide comprising:
a V_{L} CDR1 comprising the amino acid sequence KSSQNILHRTGNTYLE (SEQ ID NO: 50); a V_{L} CDR2 comprising the amino acid sequence KVSNRFS (SEQ ID NO: 7); and a V_{L} CDR3 comprising the amino acid sequence FQGSYVPFT (SEQ ID NO: 8); or
a V_{L} CDR1 comprising the amino acid sequence RSSQNILHRTGNTYLE (SEQ ID NO: 6); a V_{L} CDR2 comprising the amino acid sequence KVSNRFS (SEQ ID NO: 7); and a V_{L} CDR3 comprising the amino acid sequence FQGSYVPFT (SEQ ID NO: 8); or
a V_{L} CDR1 comprising the amino acid sequence RSSQNILHRTGNTYLD (SEQ ID NO: 53); a V_{L} CDR2 comprising the amino acid sequence KVSNRFS (SEQ ID NO: 7); and a V_{L} CDR3 comprising the amino acid sequence FQGSYVPFT (SEQ ID NO: 8).

9. The antibody of claim 7 or claim 8, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:35; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to an amino acid sequence selected from SEQ ID NO:40, SEQ ID NO:41, or SEQ ID NO: 42.

10. The antibody of claim 7 or claim 8, wherein the antibody comprises:
a variable heavy chain (VA) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:36; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to an amino acid sequence selected from SEQ ID NO:40, SEQ ID NO:41, or SEQ ID NO: 42.

11. The antibody of claim 7 or claim 8, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:38; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to an amino acid sequence selected from SEQ ID NO:40, SEQ ID NO:41, or SEQ ID NO: 42.

12. The antibody of claim 7 or claim 8, wherein the antibody comprises:
a variable heavy chain (VA) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:43; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to an amino acid sequence selected from SEQ ID NO:49, SEQ ID NO:51, or SEQ ID NO: 52.

13. The antibody of claim 7 or claim 8, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:45; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to an amino acid sequence selected from SEQ ID NO:49, SEQ ID NO:51, or SEQ ID NO: 52.

14. The antibody of claim 7 or claim 8, wherein the antibody comprises:
a variable heavy chain (VA) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:47; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to an amino acid sequence selected from SEQ ID NO:49, SEQ ID NO:51, or SEQ ID NO: 52.

15. The antibody of any one of claims 1 to 14, wherein the antibody cross-reacts with a non-human animal uPAR.

16. The antibody of claim 15, wherein the non-human animal uPAR is a non-human primate uPAR.

17. The antibody of claim 16, wherein the non-human primate uPAR is a cynomolgus uPAR.

18. The antibody of any one of claims 1 to 17, wherein the antibody is a humanized antibody.

19. The antibody of any one of claims 1 to 18, wherein the antibody is an IgG.

20. The antibody of claim 19, wherein the antibody comprises a human Fc domain.

21. The antibody of claim 20, wherein the antibody is a human IgG1.

22. The antibody of any one of claims 1 to 18, wherein the antibody is selected from the group consisting of: a Fab, a F(ab′) 2, and a F(ab′).

23. The antibody of any one of claims 1 to 18, wherein the antibody is a single chain antibody.

24. The antibody of claim 23, wherein the single chain antibody is an scFv.

25. The antibody of any one of claims 1 to 24, wherein the antibody is a bispecific antibody comprising a first antigen-binding domain comprising a V_{H} polypeptide-V_{L} polypeptide pair as defined in any one of claims 1 to 18.

26. The antibody of claim 25, wherein the bispecific antibody comprises a second antigen-binding domain that specifically binds an antigen other than uPAR.

27. A fusion protein, comprising:
a chain of an antibody of any one of claims 1 to 26 fused to a heterologous sequence of amino acids.

28. The fusion protein of claim 27, wherein the heterologous sequence of amino acids is fused to the C-terminus of the chain of the antibody.

29. The fusion protein of claim 27 or claim 28, wherein the antibody is the single chain antibody of claim 23 or 24.

30. The fusion protein of claim 29, wherein the fusion protein is a chimeric antigen receptor (CAR) comprising:
the single chain antibody;
a transmembrane domain; and
an intracellular signaling domain.

31. A conjugate, comprising:
the antibody of any one of claims 1 to 26 or the fusion protein of any one of claims 27 to 30; and
an agent conjugated to the antibody or fusion protein.

32. The conjugate of claim 31, wherein the agent is a chemotherapeutic agent, a toxin, a radiation sensitizing agent, a radioactive isotope, a detectable label, or a half-life extending moiety.

33. The conjugate of claim 32, wherein the radioactive isotope is a therapeutic radioactive isotope.

34. The conjugate of claim 32, wherein the detectable label is a radiolabel.

35. The conjugate of any one of claims 32 to 34, wherein the agent is conjugated to the antibody or fusion protein via a non-cleavable linker.

36. The conjugate of any one of claims 32 to 34, wherein the agent is conjugated to the antibody or fusion protein via a cleavable linker.

37. The conjugate of claim 36, wherein the cleavable linker is an enzyme-cleavable linker.

38. The conjugate of claim 37, wherein the linker is cleavable by a lysosomal protease.

39. The conjugate of claim 38, wherein the linker is cleavable by cathepsin or plasmin.

40. A nucleic acid encoding a variable heavy chain (VA) polypeptide, a variable light chain (V_{L}) polypeptide, or both, of an antibody of any one of claims 1 to 26.

41. A nucleic acid encoding the fusion protein of any one of claims 27 to 30.

42. An expression vector comprising the nucleic acid of claim 40 or claim 41.

43. A cell comprising the nucleic acid of claim 40 or claim 41.

44. The cell of claim 43, wherein the nucleic acid is present in an expression vector.

45. A cell comprising:
a first nucleic acid encoding the variable heavy chain (VA) polypeptide of the antibody of any one of claims 1 to 6; and
a second nucleic acid encoding the variable light chain (V_{L}) polypeptide of the antibody.

46. The cell of claim 45, comprising:
a first expression vector comprising the first nucleic acid; and
a second expression vector comprising the second nucleic acid.

47. A cell comprising an expression vector encoding the CAR of claim 30, wherein the cell expresses the CAR on its surface.

48. A method of producing the antibody or fusion protein of any one of claims 1 to 30, comprising culturing the cell of any one of claims 44 to 46 under conditions suitable for the cell to express the antibody or fusion protein, wherein the antibody or fusion protein is produced.

49. A composition, comprising:
the antibody of any one of claims 1 to 26;
the fusion protein of any one of claims 27 to 30;
the conjugate of any one of claims 31 to 39; or
a population of cells according to the cell of claim 47.

50. The composition of claim 49, wherein the antibody, fusion protein, conjugate, or population of cells is present in a liquid medium.

51. The composition of claim 49 or claim 50, comprising a pharmaceutically acceptable carrier.

52. A kit, comprising:
the composition of any one of claims 49 to 51; and
instructions for administering the composition to an individual in need thereof.

53. The kit of claim 52, wherein the composition is present in one or more unit dosages.

54. The kit of claim 52, wherein the composition is present in two or more unit dosages.

55. A method of treating a condition associated with uPAR expression and/or activity in a subject in need thereof, the method comprising administering an effective amount of the composition of any one of claims 49 to 51 to the subject.

56. The method according to claim 55, wherein the condition associated with uPAR expression and/or activity is cancer.

57. The method according to claim 56, wherein the cancer is characterized by cancer cells that express uPAR on the surface thereof.

58. The method according to claim 56 or claim 57, wherein the cancer comprises a solid tumor.

59. The method according to claim 58, wherein the cancer is characterized by stromal cells in the tumor microenvironment that express uPAR on the surface thereof.

60. The method according to claim 58 or claim 59, wherein the solid tumor is a carcinoma, lymphoma, blastoma, or sarcoma.

61. The method according to any one of claims 56 to 60, wherein the cancer is breast cancer, lung cancer, bladder cancer, ovarian cancer, prostate cancer, liver cancer, colon cancer, pancreatic cancer, gastric cancer, glioma, or any combination thereof.

62. The method according to claim 56 or claim 57, wherein the cancer comprises a hematological malignancy.

63. The method according to claim 62, wherein the hematological malignancy is a leukemia, a lymphoma, or multiple myeloma.

64. A method of inhibiting tumor invasion, tumor metastasis, degradation of extracellular matrix (ECM), tumor angiogenesis, tumor cell proliferation, or any combination thereof, in a subject having cancer, the method comprising administering an effective amount of the composition of any one of claims 49 to 51 to the subject.


SEQUENCE LISTING (55 sequences)

SEQ ID NO: 1 - protein, 126 aa, synthetic construct
QVTLKESGPGILQSSLTLSLTCSLSGFSLSTSGMGVSWIRQPSGKGLEWLAHIYWDDDKRYNPSLKTRLTISKDASRDQVFLKIISVDTADTGTYYCGRRVRNYFSGTSYWYFDVWGTGTTVTVSS

SEQ ID NO: 2 - protein, 7 aa, synthetic construct
TSGMGVS

SEQ ID NO: 3 - protein, 16 aa, synthetic construct
HIYWDDDKRYNPSLKT

SEQ ID NO: 4 - protein, 16 aa, synthetic construct
RVRNYFSGTSYWYFDV

SEQ ID NO: 5 - protein, 112 aa, synthetic construct
DVLMTQTPLSLSVSLGDQASISCRSSQNILHRTGNTYLEWYLQKPGQSPKLLIYKVSNRFSGVPDRFSGSGSGTDFTLKISRLEAEDLGVYYCFQGSYVPFTFGSGTKLEMK

SEQ ID NO: 6 - protein, 16 aa, synthetic construct
RSSQNILHRTGNTYLE

SEQ ID NO: 7 - protein, 7 aa, synthetic construct
KVSNRFS

SEQ ID NO: 8 - protein, 9 aa, synthetic construct
FQGSYVPFT

SEQ ID NO: 9 - protein, 119 aa, synthetic construct
EVQLVESGGGLVQPGESLTLSCDSNEFDFPSHDMSWVRKTPEKRLELVAAIDSDGGLTYYSNSRERRFIISRDNTKNTLYLQMRSLRSEDTALYYCARRRASYWYFDVWGTGTTVTVSS

SEQ ID NO: 10 - protein, 5 aa, synthetic construct
SHDMS

SEQ ID NO: 11 - protein, 17 aa, synthetic construct
AIDSDGGLTYYSNSRER

SEQ ID NO: 12 - protein, 10 aa, synthetic construct
RRASYWYFDV

SEQ ID NO: 13 - protein, 106 aa, synthetic construct
DILLTQSPAILPVSPGERVSFSCRASQNIGTSIHWYQQRTNGSPRLLIKYASESISGIPSRFSGSGSGTDFTLSINGVESEDIGDYYCQQSNSWPTFGGGTKLEIK

SEQ ID NO: 14 - protein, 11 aa, synthetic construct
RASQNIGTSIH

SEQ ID NO: 15 - protein, 7 aa, synthetic construct
YASESIS

SEQ ID NO: 16 - protein, 8 aa, synthetic construct
QQSNSWPT

SEQ ID NO: 17 - protein, 117 aa, synthetic construct
EVQLRQSGPELVKPGASVKISCKASGYTFTDYYMNWVKQSHEKSLEWIGNINPNNGGTDYNQKFKGKATLTVDKSSSTAYMELRSLTSEDSAVYYCARSYGSRFPYWGLGTLVTVSA

SEQ ID NO: 18 - protein, 5 aa, synthetic construct
DYYMN

SEQ ID NO: 19 - protein, 17 aa, synthetic construct
NINPNNGGTDYNQKFKG

SEQ ID NO: 20 - protein, 8 aa, synthetic construct
SYGSRFPY

SEQ ID NO: 21 - protein, 107 aa, synthetic construct
DIQMTQTTSSLSASLGDRVTISCRASQDITNYLSWYQQKPDGTVKLLIFYTAVLQSGFPLRFSGSGSGADYSLTISNLEQEDFATYFCQQGHTLPWTFGGGTKLEIK

SEQ ID NO: 22 - protein, 11 aa, synthetic construct
RASQDITNYLS

SEQ ID NO: 23 - protein, 7 aa, synthetic construct
YTAVLQS

SEQ ID NO: 24 - protein, 9 aa, synthetic construct
QQGHTLPWT

SEQ ID NO: 25 - protein, 107 aa, synthetic construct
DIQMTQTTSSLSASLGDRVTISCRASQDITNYLSWYQQKPDGTVKLLIFYTSFLQSGVPLRFSGSGSGADYSLTISNLEQEDFATYFCQQGHTLPWTFGGGTKLEIK

SEQ ID NO: 26 - protein, 7 aa, synthetic construct
YTSFLQS

SEQ ID NO: 27 - DNA, 378 bp, synthetic construct
  source 1..378: mol_type = other DNA
caggtaactctgaaagagtctggccctgggatattgcagtcctccctgaccctcagtctgacttgttctttatctgggttttcactgagcacttctggcatgggtgtgagctggattcgtcagccttcaggaaagggtctggagtggctggcacacatttactgggatgatgacaaacgctataatccatccctgaagacccggctaacaatctccaaggatgcctccagagaccaggttttcctcaagatcatcagtgtggacactgcagacactggcacatactattgtggtcgaagagttaggaattatttctccggtactagttactggtacttcgatgtctggggcacagggaccacggtcaccgtctcctca

SEQ ID NO: 28 - DNA, 336 bp, synthetic construct
  source 1..336: mol_type = other DNA
gatgttttgatgacccaaactccactctccctgtctgtcagtcttggagatcaagcctccatctcttgcagatctagtcagaacattctacatcgtactggaaacacctatttagaatggtacctgcagaaaccaggccagtctccaaagctcctgatctacaaagtttccaaccgattttctggggtcccagataggttcagtggcagtggatcagggacagatttcacactcaagatcagtagattggaggctgaggatctgggagtttattactgctttcaaggttcatatgttcctttcacgttcggctcggggacaaagttggaaatgaaa

SEQ ID NO: 29 - DNA, 358 bp, synthetic construct
  source 1..358: mol_type = other DNA
gaggtacaactggtggagtctgggggaggcttagtgcagcctggagagtccctgacactctcctgtgattccaatgaattcgatttcccttcccatgacatgtcttgggtccgcaagactccggagaagaggctggagttggtcgcagccattgatagtgatggtggtctcacctactattcaaacagtagggagagacgattcatcatctccagagacaataccaagaataccctgtaccttcaaatgagaagtctgaggtctgaggacacagccttatattactgtgcaagacggagggcctcctactggtacttcgatgtctggggcacagggaccactgtcaccgtctcctcag

SEQ ID NO: 30 - DNA, 318 bp, synthetic construct
  source 1..318: mol_type = other DNA
gacatcttgctgactcagtctccagccatcctgcctgtgagtccaggagaaagagtcagtttctcctgcagggccagtcaaaacattggcacaagtatacactggtatcagcaaagaacaaatggttctccaaggcttctcataaagtatgcctctgagtctatttctgggatcccttccagatttagtggcagtggatcagggacagattttactcttagcatcaacggtgtggagtctgaagatattggagattattactgtcaacaaagtaatagctggccaacgttcggaggggggaccaagctggaaattaaa

SEQ ID NO: 31 - DNA, 351 bp, synthetic construct
  source 1..351: mol_type = other DNA
gaggtccagctgcgacaatctggacctgagctggtgaagcctggggcttcagtgaagatatcctgtaaggcttctggatacacgttcactgactattacatgaactgggtgaaacagagccatgaaaagagccttgagtggattggaaatattaatcctaacaatggtggtactgactacaaccagaagttcaagggcaaggccacattgactgtcgacaagtcctccagtacagcctacatggagctccgcagcctgacatctgaggactctgcagtctattactgtgcaagatcttacggtagtcggtttccttactggggcctagggactctggtcactgtgtctgca

SEQ ID NO: 32 - DNA, 321 bp, synthetic construct
  source 1..321: mol_type = other DNA
gatatccagatgacacagactacatcctccctgtctgcctctctgggagacagagtaaccatcagttgcagggcaagtcaggacattaccaattatttaagttggtatcagcagaaaccagatggaactgttaaactcctgatcttttatacagcagttttacagtcaggattcccattaaggttcagtggcagtgggtctggagcagattattctctcaccattagcaacctggagcaagaagattttgccacttacttttgccaacagggtcatacgcttccgtggacgtttggtggaggcaccaagctggaaatcaaa

SEQ ID NO: 33 - DNA, 351 bp, synthetic construct
  source 1..351: mol_type = other DNA
gaggtacagctgcgacaatctggacctgagctggtgaagcctggggcttcagtgaagatatcctgtaaggcttctggatacacgttcactgactattacatgaactgggtgaaacagagccatgaaaagagccttgagtggattggaaatattaatcctaacaatggtggtactgactacaaccagaagttcaagggcaaggccacattgactgtcgacaagtcctccagtacagcctacatggagctccgcagcctgacatctgaggactctgcagtctattactgtgcaagatcttacggtagtcggtttccttactggggcctagggactctggtcactgtgtctgca

SEQ ID NO: 34 - DNA, 321 bp, synthetic construct
  source 1..321: mol_type = other DNA
gatatccagatgacacagactacatcctccctgtctgcctctctgggagacagagtcaccatcagttgcagggcaagtcaggacattaccaattatttaagttggtatcagcagaaaccagatggaactgttaaactcctgatcttttatacatcatttttacagtcaggagtcccattaaggttcagtggcagtgggtctggagcagattattctctcaccattagcaacctggagcaagaagattttgccacttacttttgccaacagggtcatacgcttccgtggacgtttggtggaggcaccaagctggaaatcaaa

SEQ ID NO: 35 - protein, 117 aa, synthetic construct
EVQLVQSGAEVKKPGATVKISCKVSGYTFTDYYMNWVQQAPGKSLEWMGNINPNNGGTDYNQKFQGRVTITADKSTSTAYMELSSLRSEDTAVYYCARSYGSRFPYWGLGTLVTVSS

SEQ ID NO: 36 - protein, 117 aa, synthetic construct
EVQLVQSGAEVKKPGASVKVSCKASGYTFTDYYMNWVRQAPGKSLEWMGNINPNNGGTDYSQKFQGRVTITVDTSASTAYMELSSLRSEDTAVYYCARSYGSRFPYWGLGTLVTVSS

SEQ ID NO: 37 - protein, 17 aa, synthetic construct
NINPNNGGTDYSQKFQG

SEQ ID NO: 38 - protein, 117 aa, synthetic construct
EVQLVQSGAEVKKPGASVKVSCKASGYTFTDYYMNWVRQAPGKSLEWMGNINPNNGGTDYNQKFQGRVTLTVDTSISTAYMELSRLRSDDTVVYYCARSYGSRFPYWGLGTLVTVSS

SEQ ID NO: 39 - protein, 17 aa, synthetic construct
NINPNNGGTDYNQKFQG

SEQ ID NO: 40 - protein, 107 aa, synthetic construct
DIQMTQSPSSLSASVGDRVTITCRASQDITNYLSWYQQKPGGTVKLLLYYTAVLQSGFPSRFSGSGSGADYTLTISSLQPEDFATYYCQQGHTLPWTFGGGTKVEIK

SEQ ID NO: 41 - protein, 107 aa, synthetic construct
DIQMTQSPSSLSASVGDRVTITCRASQDITNYLSWYQQKPGGTVKLLIFYTAVLQSGFPSRFSGSGSGADFTLTISSLQPEDVATYYCQQGHTLPWTFGGGTKVEIK

SEQ ID NO: 42 - protein, 107 aa, synthetic construct
DIQMTQSPSSLSASVGDRVTITCRASQDITNYLSWFQQKPGGTVKLLIYYTAVLQSGFPSRFSGSGSGADFTLTISSLQPEDFATYYCQQGHTLPWTFGGGTKVEIK

SEQ ID NO: 43 - protein, 126 aa, synthetic construct
QVTLRESGPALVKPTQTLTLTCTLSGFSLSTSGMGVSWIRQPPGKALEWLAHIYWDDDKRYSTSLKTRLTISKDASKDQVVLTMTNMDTVDTATYYCGRRVRNYFSGTSYWYFDVWGTGTLVTVSS

SEQ ID NO: 44 - protein, 16 aa, synthetic construct
HIYWDDDKRYSTSLKT

SEQ ID NO: 45 - protein, 126 aa, synthetic construct
QVTLKESGPVLVKPTETLTLTCTVSGFSLSTSGMGVSWIRQPPGKGLEWLAHIYWDDDKRYSPSLKSRLTISKDASKDQVVLTMTNMDTVDTATYYCGRRVRNYFSGTSYWYFDVWGTGTLVTVSS

SEQ ID NO: 46 - protein, 16 aa, synthetic construct
HIYWDDDKRYSPSLKS

SEQ ID NO: 47 - protein, 126 aa, synthetic construct
QITLKESGPTLVKPTQTLTLTCTLSGFSLSTSGMGVSWIRQPPGKALEWLAHIYWDDDKRYNPSLKSRLTITKDASKDQVVLTMTNMDTVDTATYYCGRRVRNYFSGTSYWYFDVWGTGTLVTVSS

SEQ ID NO: 48 - protein, 16 aa, synthetic construct
HIYWDDDKRYNPSLKS

SEQ ID NO: 49 - protein, 112 aa, synthetic construct
DIVMTQTPLSLSVTPGQPASISCKSSQNILHRTGNTYLEWYLQKPGQPPQLLIYKVSNRFSGVPDRFSGSGSGTDFTLKISRVEAEDVGVYYCFQGSYVPFTFGQGTKLEIK

SEQ ID NO: 50 - protein, 16 aa, synthetic construct
KSSQNILHRTGNTYLE

SEQ ID NO: 51 - protein, 112 aa, synthetic construct
DVVMTQSPLSLPVTLGQPASISCRSSQNILHRTGNTYLEWFQQRPGQSPRLLIYKVSNRFSGVPDRFSGSGSGTDFTLKISRVEAEDVGVYYCFQGSYVPFTFGQGTKLEIK

SEQ ID NO: 52 - protein, 112 aa, synthetic construct
DIVMTQSPLSLPVTPGEPASISCRSSQNILHRTGNTYLDWYLQKPGQSPQLLIYKVSNRFSGVPDRFSGSGSGTDFTLKISRVEAEDVGVYYCFQGSYVPFTFGQGTKLEIK

SEQ ID NO: 53 - protein, 16 aa, synthetic construct
RSSQNILHRTGNTYLD

SEQ ID NO: 54 - protein, 15 aa, synthetic construct
GGGGSGGGGSGGGGS

SEQ ID NO: 55 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT